(19) United States
(12) Patent Application Publication
(10) Pub. No.: US 2005/0209614 A1
(43) Pub. Date: Sep. 22, 2005

(54) Anastomosis apparatus and methods with computer-aided, automated features

(76) Inventors: Felix West Fenter, Irving, TX (US); William C. Garre, Dallas, TX (US)

Correspondence Address: SHERMAN & SHALLOWAY, 415 NORTH ALFRED STREET, ALEXANDRIA, VA, 22314, US

(21) Appl. No.: 10/791,733

(22) Filed: Mar. 4, 2004

(51) Int. Cl. A61B 17/08

(52) U.S. Cl. 606/153

(57) ABSTRACT

The present invention provides an apparatus and method for performing precision, positive, and permanent anastomosis of blood vessels which makes use of miniature appliance/applicators that execute the anastomosis procedures under the control of the surgeon and the surgical team. The appliance/applicators are constructed in various sizes and configurations to accommodate the wide variations in size of the tubular body members and the various anastomosis types (end-to-end, end-to-side, and side-to-side joining). The apparatus features a surgeon's manipulator wand to which the surgeon attaches the appropriate type and size appliance/applicator. Using the wand, the surgeon positions and actuates the applicator which then automatically performs several sequential steps to attach the appliance and effect a precise and uniform anastomosis of the tubular members. The surgeon's wand contains the actuators, servos, and sensors that operate the appliance/applicator in response to commands from the surgeon. The appliance portion of the appliance/applicator consists of a plurality of connectable grippers designed for the particular type of anastomosis being performed and remains in the patient as a permanent part of the joined tubular members, with the applicator part being withdrawn and discarded after the procedure is complete. The apparatus further comprises a support and positioning arm assembly, a mobile console housing the various elements and power source and a computer providing automated control of the apparatus and process as well as inventory of the appliance/applicator kits.

### BACKGROUND OF THE INVENTION

1. Field of the Invention

This invention relates generally to the field of surgery in which a tubular body member (e.g., a vein or artery) is to be connected to a second body member, the second member being another tubular member of a similar size, a smaller size, or a relatively large member such as the aorta of the heart. More specifically, it relates to an appliance which is used by the surgeon in making the desired connection, and a computer-controlled apparatus that actually accomplishes the connecting step under the direction of the surgeon using a hand-controlled manipulator wand. The goal of the invention is to make significant improvements over the anastomosis methods of the prior art, which include sewing (suturing) as well as the use of staples and clips.

2. Background of Related Art

Surgical procedures have used anastomosis methods successfully for the last 100 years. However, the methods remain crude, are prone to leakage, can cause damage to the endothelium at the joint, and are time-consuming to perform. Also, in many operations, the anastomosis procedures are very difficult and fatiguing, even for the most skilled surgeons, because of access limitations and/or the small size of the vessels requiring anastomosis.

Vascular surgeons perform anastomoses almost exclusively by traditional manual suturing; consequently, surgeons are required to have great physical stamina and endurance as well as outstanding manual skill and dexterity. These tedious manual procedures considerably extend operating room time, surgeon fatigue and patient risk. Additional problems associated with manual suturing include the inevitable damage to the blood vessel intima due to needle/suture puncture as well as necrosis of the vessel tissue adjacent to each suture. Damage to the intima can cause the deposition of platelets and other thrombotic materials at each penetration site. Necrosis of the vessel tissue can hinder the healing process and can result in a reduction of the structural integrity of the blood vessel.

Recently, many other approaches have appeared that involve using metallic staples, pins or clips instead of sutures. Aside from somewhat ameliorating the endurance and dexterity requirements, staples and pins have all the same disadvantages as sutures while clips tend to cause excessive necrosis at each clip location.

During recent years, the number of operations requiring anastomosis has increased significantly. A primary reason is the pervasiveness of cardiovascular disease in the United States and other developed countries. In addition, due to the increasing skill of the surgeons, new operating room equipment and devices, and improvement in surgical procedures, the success ratio for cardiovascular operations and organ transplant operations has stimulated a dramatic increase in the number of operations, each requiring numerous and difficult anastomosis procedures. For example, in the United States alone, over 600,000 coronary bypass operations are performed each year, and this number is increasing rapidly. Consequently, it is imperative that improved anastomosis methods be developed and proven as soon as possible.

Various instruments are known in the prior art for end-to-end and end-to-side anastomotic surgical stapling together of parts of the alimentary canal (i.e., esophagus, stomach, colon, etc.). These instruments employ staple cartridges, generally in the shape of a hollow cylinder, of different sizes to accommodate tubular organs of varying diameters. End-to-end and end-to-side anastomoses are achieved by means of at least one ring of surgical staples. The traditional technique for surgical stapling anastomosis is to position the stapling cartridge within the tubular organ to be stapled. The cut end of the tubular organ is inverted (i.e., folded inwardly) over the annular end of the staple cartridge creating an inverting anastomosis upon stapling. An essential requirement of the inverting anastomotic technique is the incorporation of knives within the staple cartridge housing to trim excess tissue from the anastomotic connection.

Representative of such devices are those disclosed in U.S. Pat. No. 5,250,058, Miller, et al., U.S. Pat. No. 5,697,943, Sauer, et al., and U.S. Published Patent Application No. 2002/0082625, Huxel, et al.

The prior art anastomotic stapling instruments form generally circular anastomotic connections, and have been largely limited to alimentary organs. With respect to end-to-side vascular anastomosis, circular connections, rather than elliptical connections, are sometimes disadvantageous as they are less physiologic or natural. Representative of devices employed in end-to-side vascular anastomoses producing a circular connection are U.S. Pat. No. 5,833,698, Hinchliffe, et al., U.S. Pat. No. 6,440,146, Nicholas, et al., and U.S. Pat. No. 6,428,550, Vargas, et al. This unnatural connection may create turbulence in the blood flow as it courses through the anastomosis, damaging the intima (i.e. inner wall) of the blood vessel and predisposing it to forming blood clots. In the present state of the art, end-to-end and end-to-side anastomosis between blood vessels have typically been accomplished by hand-sewn suturing techniques occasionally employing a connection or suturing aid such as those represented by U.S. Pat. No. 3,254,651, Collito, and U.S. Pat. No. 3,561,448, Petemel. These techniques are time consuming, not as reliable as stapling, and subject to greater human error than stapling. Current stapling instruments used for alimentary canal applications are not suitable, however, for vascular anastomosis due to their large sizes and inability to provide non-circular and low turbulence anastomoses. A typical prior art instrument has a circumference of approximately 8 cm (3 in), far too thick to accommodate coronary arteries and veins, which have circumferences ranging from 0.50 to 1.0 cm and from 1.5 to 2.5 cm, respectively.

An additional drawback of prior stapling instruments is the inability to provide an everted (i.e., folded outwardly) anastomosis. An inverted vascular anastomosis, such as that resulting from the use of devices and methods disclosed in U.S. Pat. No. 4,294,255, Geroc, U.S. Pat. No. 5,250,058, Miller, et al., U.S. Pat. No. 5,336,233, Chen, and U.S. Published Patent Application 2002/0082625, Huxel, et al., would expose the cut ends of the blood vessels to the vessel lumen and could lead to the formation of blood clots. For this reason, hand-sewn everted anastomoses for vascular connections are preferable, despite time and reliability drawbacks. Attempts have been made to provide instruments capable of creating an everted and stapled anastomosis, such as U.S. Pat. No. 4,523,592, Daniel, U.S. Pat. No. 4,917,091, Berggren, et al., U.S. Pat. No. 4,971,090, Berggren, et al., U.S. Pat. No. 6,428,550, Vargas, et al. and U.S. Published Patent Application 2002/0058955, Blatter, et al. However, these devices have drawbacks to achieving a uniform and secure anastomosis in that the tissue of the vessels being joined must be manually stretched over a connector structure which often results in an uneven tension around the anastomosis. Accordingly, it is a general object of the present invention to provide an improved instrument and method for vascular anastomosis. It is also an object of the present invention to provide an automated surgical anastomotic device small enough to accommodate vascular lumens.

Another objective of the present invention is to provide a computer controlled device for everted anastomosis. An additional object of the present invention is to provide a method for surgical anastomosis that does not require the removal of excess tissue from the anastomotical connection. Still another objective of the present invention is to provide an instrument and method for vascular anastomosis that is less time-consuming and more reliable than the prior art. At present, essentially all vascular anastomoses are performed by conventional hand suturing due to the deficiencies and drawbacks of the prior art devices. Suturing an anastomosis is a time-consuming and difficult task, requiring much skill and practice on the part of the surgeon. It is important that each anastomosis provide a smooth, open flow path for the blood and that the attachment be completely free of leaks. A completely leak-free seal is not always achieved on the first attempt. Consequently, there is a frequent need for resuturing of the anastomosis.

### SUMMARY OF THE INVENTION

The design of an automated anastomosis system requires the establishment of a set of minimum requirements to provide a design guide. These requirements have been defined in cooperation with a medical advisory board and cover a wide spectrum of anastomosis procedures. The effect of these requirements on the system appears either directly or indirectly in the descriptions of the Surgical Automaton System (SAS), the Appliance, the Applicator and their components.

The design requirements, goals or criteria for the automated anastomosis Appliance are defined as follows:

Minimal or no disturbance of the endothelium layer to avoid or minimize the deposition of platelets and other thrombotic materials on the inner surfaces of the anastomosed vessels.

No requirement for clamps, clips, pins or staples that cause excessive local necrosis.

No requirement for a cardiopulmonary by-pass machine.

No requirement to stop the heart.

All materials to be used and left in the body must be compatible with living tissue.

The induced blood vessel strains caused by the eversion process should not exceed 30%.

No reduction of patency should be induced by the Appliance.

The Appliance should permit future blood vessel growth.

The Appliance/Applicator (A/A) must be small with minimal access needs.

The application is quick, accurate and highly automated.

The Applicator is low cost and disposable.

The Applicator actions should be driven by micro-electro-mechanical actuators or other suitable actuation means.

The blood vessel exposure and preparation should be accomplished by traditional means.

The final automated anastomosis should provide the means for examination, repair and/or reinforcement with conventional sutures if deemed necessary by the surgeon.

The present invention makes use of miniature Appliance/Applicators (A/A's) that execute the anastomosis procedures under the control of the surgeon and the surgical team. The A/A's are constructed in various sizes and configurations to accommodate the wide variations in size of the tubular body members and the various anastomosis types (end-to-end, end-to-side, and side-to-side joining). The system features a surgeon's manipulator wand to which the surgeon attaches the appropriate type and size A/A. Using the wand, the surgeon positions and actuates the applicator which then automatically performs several sequential steps to attach the appliance and effect a precise and uniform anastomosis of the tubular members. The surgeon's wand contains the actuators, servos, and sensors that operate the A/A in response to commands from the surgeon. The appliance portion of the A/A remains in the patient as a permanent part of the joined tubular members, and the applicator part of the A/A is withdrawn and discarded after the procedure is complete. The surgeon's wand, and in turn, the A/A, is operated by the surgeon via a voice-controlled, computer-driven, intelligent “surgeon's assistant” system. The system also may be controlled manually by the surgeon; in this mode the system still provides the computer-driven, intelligent features and the automatic performance of sequential steps to effect a precise and uniform anastomosis.

Key equipment items of the system are housed in a mobile console which is placed adjacent to the operating table, positioned by the surgeon and surgical team for maximum efficiency and accessibility in performing the necessary anastomosis procedures. The mobile console is configured with an extendable mast and articulated arms, and the surgeon's wand is attached to the end of the working arm mechanism. This provides the “reach” necessary for the surgeon to perform anastomosis procedures at any desired location on the operating table.

The present invention provides a precision, positive, and permanent anastomosis which is less likely to experience leakage than are the other methods currently in use. The invention reduces the time required to accomplish each anastomosis procedure, yielding significant benefits by reducing surgeon and surgical team fatigue and by reducing the total time for the operation. Another significant benefit of this invention is that the endothelium layers are placed in apposition at the joint, and the joining devices do not penetrate the inner surfaces of the vessels being joined. These features serve to minimize damage to the internal surfaces of the vessels in the vicinity of the anastomosis and thereby reduce the potential for formation of blood clots.

With the foregoing in mind, it is an object of the present invention to provide a precision surgical appliance for gripping and manipulating tubular vessel structures, such as blood vessels, that have been exposed and prepared for permanent joining (anastomosis). The appliance grips the subject vessel near its prepared edge by means of a multiplicity of sharpened, miniature tines made of stainless steel or other suitable material which precisely, partially, and permanently penetrate and extend into the wall of the vessel without disturbing the sensitive intima on the interior of the vessel wall. These tines are held and guided by a suitably shaped annular collar or individual segments made of silicone, polypropylene, or other suitable material. Thus, the appliance provides a means for holding, distending, everting, and/or otherwise distorting the prepared edge of the vessel as well as a means for permanently holding the prepared edge or the everted interior of the vessel in apposition to another prepared vessel similarly equipped with a gripping appliance.

It is a further object to provide a positive, flexible, miniature anastomosis appliance to accomplish the permanent joining of tubular vessels without penetrating the endothelium, with a minimum disruption of the subsequent fluid flow inside the joined vessels and with minimum or no necrosis in the tissue near the anastomosis.

It is a still further object to provide a miniature, disposable applicator device which is configured to position, control, manipulate, distort, and join anastomotic appliance components in a programmed sequential manner to effect the anastomosis of prepared tubular structures at the command of a surgeon and without further human intervention once the automated joining procedure has been initiated.

It is an even further object to provide a properly shaped expandable sub-component of an applicator which can be inflated to various degrees in a controlled manner with a suitable liquid (such as a saline solution) so as to automatically align, distend, and/or evert the vessel surfaces to be joined without damaging or otherwise harming the intima of the vessels being joined.

It is a still further object to provide a system which can be equipped with appropriate appliances and applicators, positioned appropriately adjacent to anastomotically prepared vessel edges and commanded to automatically, and without further human intervention, accomplish a variety of anastomosis procedures using preprogrammed computer logic and protocols.

It is also an object to provide a system which, when equipped with appropriate appliances and applicators, can automatically accomplish the anastomotic end-to-end, end-to-side, and/or side-to-side joining of two properly prepared tubular vessels with access limited to only the mating end or mating side of each vessel being joined.

And it is an object to provide software which allows a preprogrammed computer to operate the applicator actuators, displays, and all other elements of the system in a logical, safe, controlled, auditable, reliable, and medically sound manner.

Further objects and advantages will be evident form the following drawing figures, description and claims.

### BRIEF DESCRIPTION OF THE DRAWINGS

FIGS. 1A-C are representations of Type I end-to-end, Type II side-to-side and Type III end-to-side anastomoses, respectively.

FIG. 2 is a schematic representation of a type I end-to-end anastomosis.

FIG. 3A is a schematic cross section of blood vessels partially everted for a Type I anastomosis.

FIG. 3B is a schematic cross section of blood vessels in apposition with intima-to-intima contact in a Type I anastomosis.

FIG. 4A is a schematic cross section of blood vessels partially everted for a Type II anastomosis.

FIG. 4B is a schematic cross section of blood vessels in apposition with intima-to-intima contact in a Type II anastomosis.

FIG. 5A is a schematic cross section of blood vessels partially everted for a Type III anastomosis.

FIG. 5B is a schematic cross section of blood vessels in apposition with intima-to-intima contact in a Type III anastomosis.

FIG. 6A is a graph showing the strain to rupture relationship of everted arteries and veins.

FIG. 6B illustrates a partially everted blood vessel.

FIG. 6C illustrates a fully everted blood vessel.

FIG. 7 is a graph showing the approximate normalized strain due to partial eversion.

FIG. 8 is a graph showing the approximate effect of blood vessel cut-off angle on the strain due to eversion.

FIG. 9A illustrates a planar arterio/venotomy for approximate calculation of eversion strain.

FIGS. 9B and C are graphs relating eversion dimensions of an arterio/venotomy to strain.

FIGS. 10A-C illustrate the general configuration of the Surgical Automaton System (SAS) of the present invention in transport (10A & B) and operational (10C) configurations.

FIG. 11 is a block diagram of the operations of the five subsystems of the SAS.

FIG. 12 is a top view of the surgeon's wand portion of the SAS.

FIG. 13 is a side view of the surgeon's wand.

FIG. 14 is a schematic layout of the operational elements of the surgeon's wand with enlarged views of the transmission gears and applicator drive gears.

FIG. 15 illustrates the transmission and applicator drive gear assemblies within the surgeon's wand including end views thereof showing the relationship from one end of the wand to the other.

FIG. 16 illustrates side and end views of the transmission gears showing the seven position translating drive pinion and snubber assembly.

FIG. 17 illustrates alternative stylus types for the surgeon's wand.

FIG. 18 illustrates the general operation of the SAS working arm.

FIG. 19 illustrates the conceptual balancing principles for the variable length of the working arm.

FIG. 20 illustrates the working arm footprint for a specific SAS cart location.

FIGS. 21A-C illustrate the components of a Type I PAG assembly according to the present invention.

FIGS. 22A-E illustrate the sequence of operations leading to eversion of a blood vessel end in a Type I anastomosis according to the present invention.

FIGS. 23A and B illustrate the sequence of formation of a Type I anastomosis according to the present invention.

FIGS. 24A and B illustrate release of the PAG holder/ejector assemblies of the present invention following completion of a Type I anastomosis.

FIGS. 25A and B illustrate a completed Type I anastomosis according to the present invention with FIG. 25B being a longitudinal cross section of FIG. 25A.

FIG. 26 is a chart of the steps in performing a Type I anastomosis according to the present invention keyed to the transmission drive of the apparatus.

FIGS. 27A-H illustrate the components of a Type I appliance/applicator of the present invention through its operation in accordance with the steps of FIG. 26.

FIGS. 28A-D illustrate alternative connector structures for a Type I PAG of the present invention.

FIG. 29 is an exploded view of the major components of a Type III PAG assembly according to the present invention.

FIG. 30 illustrates the Type III PAGs according to the present invention.

FIGS. 31A and B illustrate the Type III side graft PAG holder of the present invention in closed and open configuration.

FIG. 31C is a cross section of a Type III side graft PAG in its holder prior to connection with a cooperating end graft PAG and subsequent ejection.

FIG. 31D is a cross section of a Type III side graft PAG connected to a Type III end graft PAG and ejected from its holder.

FIGS. 32A and B illustrate the Type III end graft PAG holder of the present invention in closed and open configuration.

FIG. 32C is a cross section of a Type III end graft PAG in its holder prior to connection with a cooperating side graft PAG and subsequent ejection.

FIG. 32D is a cross section of a Type III end graft PAG connected to a Type III side graft PAG and ejected from its holder.

FIG. 33A illustrates the preparation of a side graft blood vessel for anastomosis.

FIG. 33B illustrates the presentation of the Type III appliance/applicator of the present invention to the side graft vessel.

FIGS. 34A-E illustrate the steps of preparing the side graft vessel for eversion using the Type III appliance/applicator of the present invention.

FIGS. 35A-F illustrate the steps of eversion of the side graft vessel wall and engagement of the side graft PAGs of the present invention.

FIG. 36 illustrates the insertion of the trimmed side graft vessel into the Type III appliance/applicator of the present invention for eversion and anastomosis.

FIGS. 37A-D illustrate the end graft PAG holder ejector assembly and everter of the present invention in condition to receive the end graft vessel, FIG. 37A being a top view,

FIG. 37B being a longitudinal cross section, FIG. 37C being a horizontal cross section and

FIG. 37D being a horizontal cross section with the end graft vessel in place.

FIGS. 38A-D illustrate the steps of eversion of the end graft vessel and engagement of the end graft PAGs of the present invention.

FIGS. 39A-C illustrate the steps of completion of the Type III anastomosis.

FIGS. 40A and B illustrate a completed end Type III anastomosis according to the present invention with FIG. 40B being a cross section thereof.

FIG. 41 is a chart of the steps in performing a Type III anastomosis according to the present invention keyed to the transmission drive of the apparatus.

FIG. 42 is an exploded view of an appliance/applicator kit of the present invention.

FIG. 43 is a view of an operating room illustrating alternative embodiments of the Surgical Automaton System (SAS) of the present invention.

FIG. 44 is a schematic layout of the operational elements of an alternative embodiment of the surgeon's wand.

FIG. 45 is a view of the appliance/applicator interface of the alternative embodiment of FIG. 44.

FIG. 46 is a partial schematic of an alternative embodiment of the appliance/applicator used with the surgeon's wand of FIG. 44.

### DETAILED DESCRIPTION OF THE INVENTION

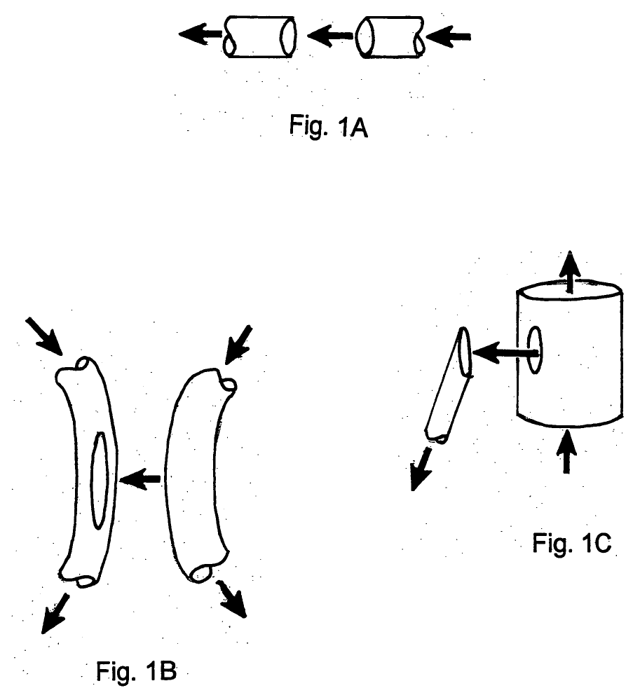

Turning now to the drawings, there are three basic types of anastomosis procedures that would be performed using the SAS A/A kits. The three types are illustrated in FIG. 1A-1C and are, end-to-end, side-to-side and end-to-side, respectively. In all the figures and in this discussion, they are referred to as Type I, Type II and Type III, respectively. It can be seen that Type III is essentially a hybrid combination of Type I and Type II; however, it is included here as a basic type because of its importance and prevalence in coronary by-pass procedures. FIG. 1 includes arrows indicating the direction of blood flow. It is important to understand that the functioning of the SAS system is not materially affected by the direction of blood flow in the vessels. The arrows shown merely represent one possible arrangement of the proximal and distal components of the graft. In many of the subsequent figures, the vessels to be joined are identified as proximal and/or distal, but this is done only as a means of identifying each vessel for the purpose of discussion, and the anastomosis process itself is not affected.

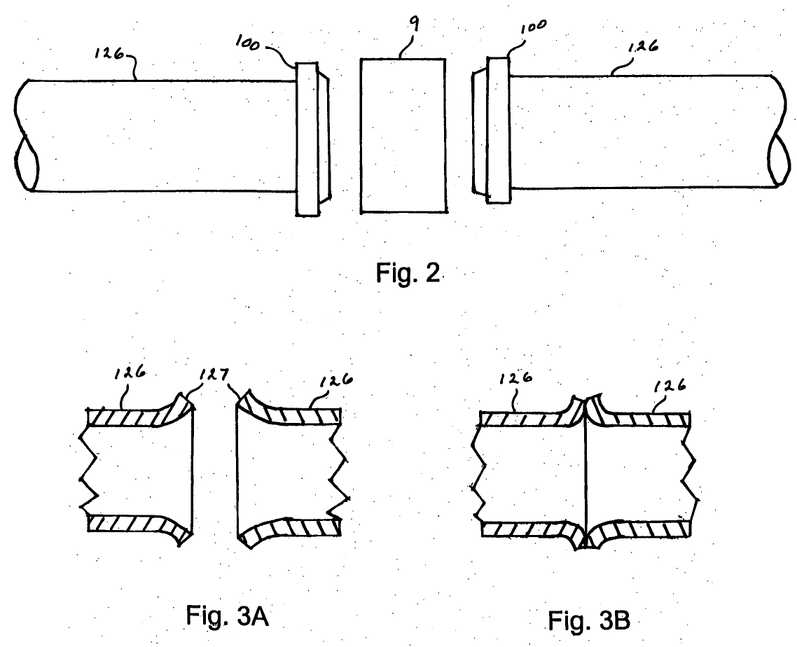

The components that remain in the body after a Type I anastomosis procedure are two sets of precision anastomosis grippers (PAGs) 100 and their connection means and are shown in schematic form in FIG. 2. These components are referred to as the appliance. The PAGs 100 grip and shape the prepared ends of the blood vessels 126, 230 and 231 with no penetration of the endothelium layer of the blood vessel. When the two sets of PAGs 100 are connected, the blood vessel walls are held in apposition with intima-to-intima contact.

All the actions of the appliance components such as (but not limited to) attaching, positioning, and manipulating are performed by a small disposable applicator 9 (to be discussed later). The applicator and its various actuators and motions are controlled and sequenced by a computer system. The computer system is controlled by the surgeon using a small set of generic commands.

The ideal configuration of the blood vessels for a Type I anastomosis is shown in FIG. 3. The exposed and trimmed ends 127 of the vessels 126 to be joined should be partially everted as shown in FIG. 3A and then held in apposition with intima-to-intima contact, as shown in FIG. 3B, to provide a hemodynamic seal. The intima-to-intima contact also serves to avoid or minimize the thrombogenicity associated with exposing the blood flow to non-intimal materials. The means for achieving the partial eversion and the means for maintaining the intima-to-intima contact are not shown in this idealized depiction.

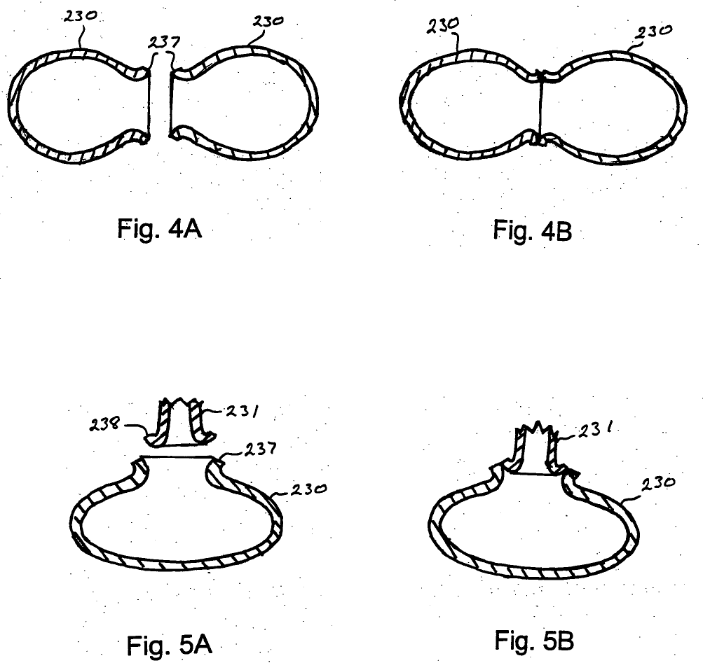

The ideal configuration of the blood vessels for a Type II anastomosis is shown in FIG. 4. The vessels 230 must be exposed and then subjected to arteriotomies (or venotomies) to provide the desired passageway between the two vessels 230. The edges 237 of the arteriotomies/venotomies are partially everted, as shown in FIG. 4A, and then held in apposition with intima-to-intima contact, as shown in FIG. 4B.

All the comments concerning the Type I anastomosis in FIG. 3 apply equally to the Type II anastomosis shown here.

The ideal configuration of the blood vessels for a Type III anastomosis is shown in FIG. 5. The vessels 230 and 231 must be exposed and the end graft vessel 231 trimmed to the angle desired for the anastomosis. A matching arteriotomy/venotomy is then performed on the side graft vessel 230. The edges 238 and 237 of the end graft and the arteriotomy/venotomy are partially everted, FIG. 5A, and held in apposition with intima-to-intima contact, FIG. 5B.

All the comments concerning the Type I anastomosis in FIG. 3 apply equally to the Type III anastomosis shown here.

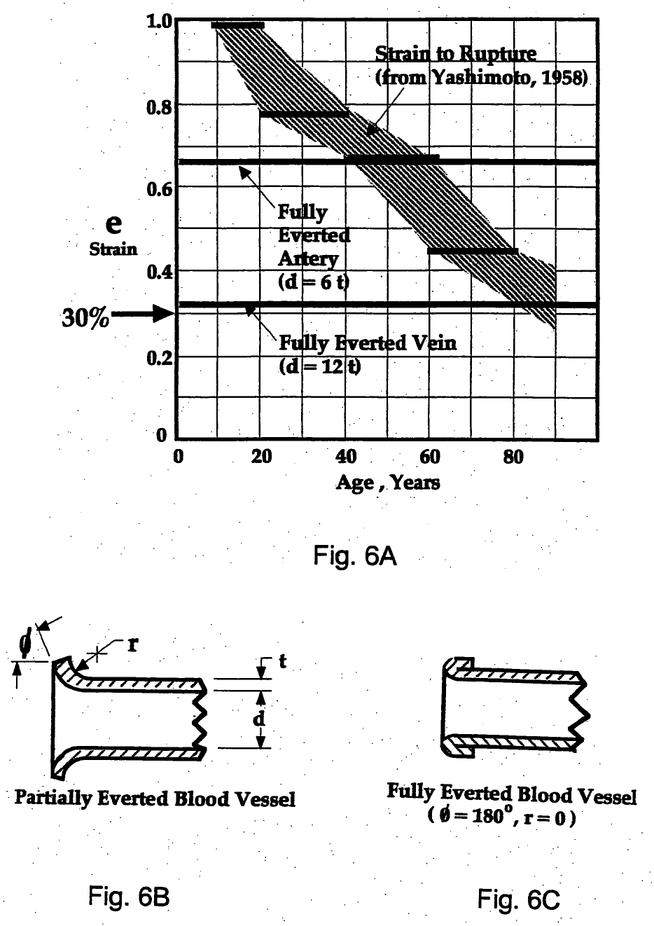

The ability of a blood vessel to be stretched without rupture depends upon the age and the vascular health of the patient as indicated by the experimental data reported by Yashimoto and many others. The data plotted in the graph of FIG. 6A clearly indicate why a maximum strain of 30% was chosen as a design goal. This conservative approach also permits the use of convenient, approximate analysis methods for calculating strains. This design goal is obviously a very important one although in many cases it greatly complicates the design process.

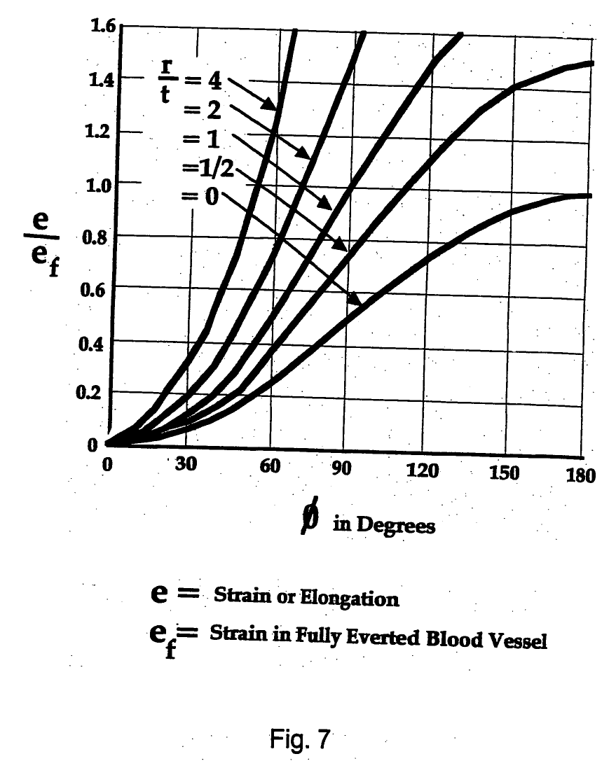

FIG. 6B also defines the partial eversion process in terms of the lumen diameter (d), the wall thickness (t), the radius of curvature of the stretched end of the vessel (r) and the angle through which the end is stretched (φ). The special case of a fully everted vessel, FIG. 7C, is defined as r=0 and φ=180 degrees. The approximate strains induced in typical fully everted arteries and veins, FIG. 6C, are shown on the graph. A typical fully everted artery experiences a maximum strain of approximately 67% while a typical fully everted vein experiences a maximum strain of approximately 33%. It is clear that any device or procedure that involves fully everting an artery should be regarded as extremely dangerous particularly when applied to more mature patients.

The normalized eversion strain, i.e. the ratio of strain to fully everted strain, as a function of the eversion angle and the ratio of eversion radius to blood vessel thickness is plotted in the graph of FIG. 7. The graph shows the normalized eversion strain for a number of eversion radius-to-thickness ratios. This graph is important in identifying the desired eversion angle and, in combination with the discussion in FIG. 6, to assess if the eversion will be in a dangerous zone.

Assuming that the eversion radius is twice the blood vessel thickness (r/t=2), the graph shows that an eversion of 60 degrees will yield a normalized strain of approximately 75% of the fully everted strain. Knowing the fully everted strain as a function of the lumen diameter and the wall thickness of the blood vessel, one can calculate the approximate induced strain at this eversion angle. Then, this strain is used in FIG. 6 along with the age of the patient to identify if the patient is in or near the dangerous rupture zone.

Another interpretation of this graph is that as the eversion radius to wall thickness ratio increases, the eversion angle for the same normalized strain decreases. For example, for a normalized strain ratio of 0.4 units, the eversion angle is approximately 35, 45, 55, 60 and 80 degrees for eversion radius-to-thickness ratios of 4, 2, 1, 1/2 and 0, respectively.

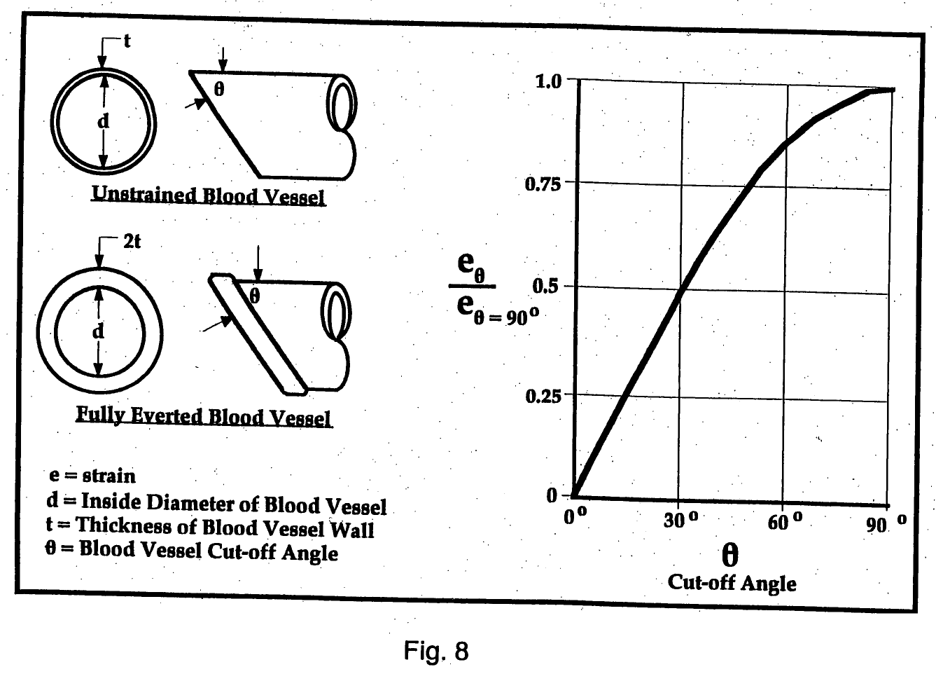

The cut-off angle of vessels being anastomosed is another factor in determining the strain applied due to eversion. The cut-off angle for Type I anastomosis is normally 90 degrees; however, smaller cut-off angles are sometimes used to reduce the strain for a given eversion angle. The graph in FIG. 8 shows that the cut-off angle is a powerful design tool for avoiding excessive strains.

In the case of Type III (end-to-side) anastomoses, the end of the side graft vessel is usually cut at an angle considerably less than 90 degrees because of space limitations within the patient's body. This, of course, also reduces the strain due to eversion in the side graft vessel. For example, the graph shows that a cut-off angle of 30 degrees reduces the eversion strain to 50% of the strain in a 90 degree graft.

This graph can be used along with the graph in FIG. 7 to determine the combinations of cut-off angle, eversion angle and eversion radius that will result in strains less than 30% for a given blood vessel lumen diameter and wall thickness.

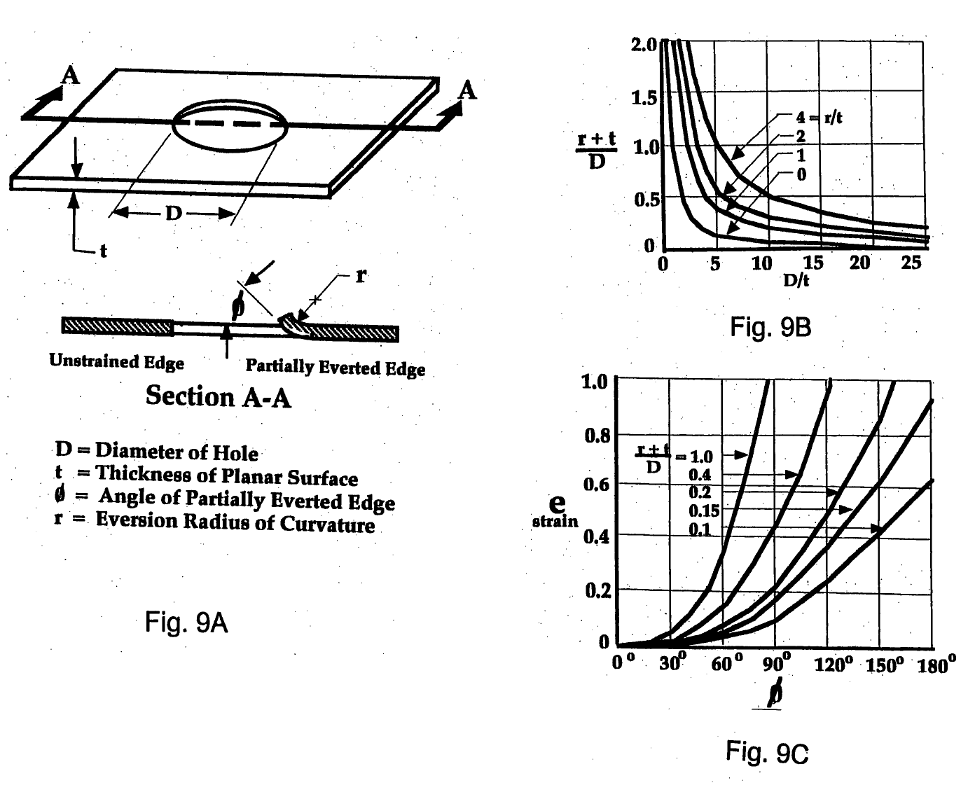

The Type II and Type III anastomoses require the eversion of the edge of a hole in the side wall of a blood vessel, unlike Type I which requires the eversion of the vessel ends only. The strain due to eversion of the edge of an arteriotomy/venotomy in the side wall of a blood vessel can be estimated by analyzing a hole in a planar surface as indicated in FIG. 9A. The analysis assumes that the edge of a circular hole of diameter, D, is to be everted on the locally planar side of a blood vessel. The blood vessel is assumed to have thickness t, the eversion radius is r and the eversion angle is φ. The results of the analysis are shown in the graphs in FIGS. 9B and 9C.

These graphs can be used to estimate the eversion strain for a circular hole in the locally planar side of a blood vessel. The graphs can also be used to roughly estimate the eversion strain for a non-circular hole if the shape of the hole is smooth (an ellipse, for example), and D is defined as the diameter of a circle whose circumference is numerically the same as the perimeter of the actual hole.

The foregoing calculations and graphs make it possible for a surgeon to calculate the strain level that will be placed on a blood vessel at the point of anastomosis prior to surgery. In addition, the degree of eversion that the vessels will withstand may also be readily determined and, thereby, the likely success of the anastomosis.

As previously noted, it is preferred that the ends of vessels to be joined by anastomosis be everted so as to bring the endothelial layers into direct contact and avoid exposure of the cut ends of the vessels to the vessel lumen. Achieving this eversion to the precise degree necessary to result in endothelial contact without placing excessive strain on the vessels is difficult when performed manually by suturing or with prior art devices.

The present invention provides an apparatus and method whereby the eversion and anastomosis may be performed efficiently, without undue strain on the vessels being joined, and in less time and with better precision than manual suturing.

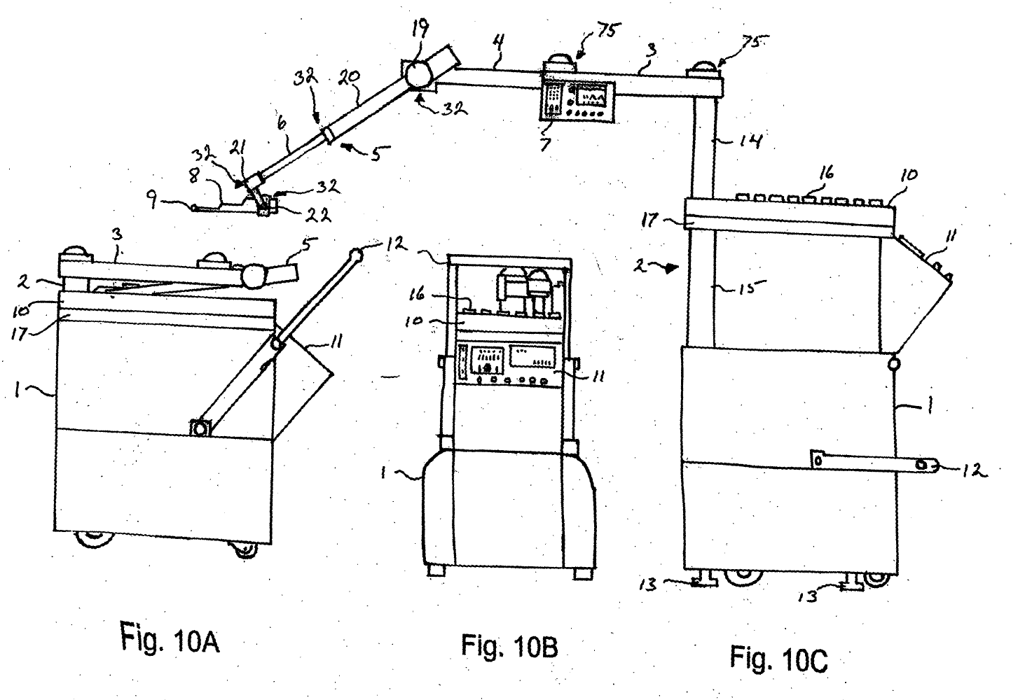

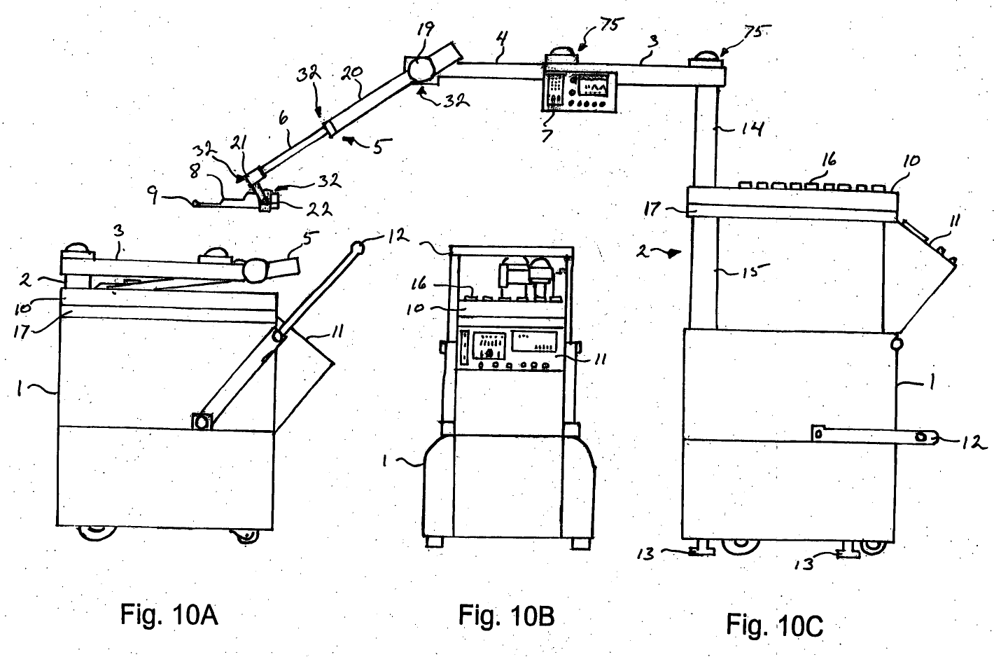

The surgical automaton system, SAS, of the present invention is depicted in FIGS. 10A, 10B and 10C. The main components of SAS are a mobile transport cart 1, a mast assembly 2, a positioning arm 3 with a sliding segment 4, a working arm 5 with a sliding segment 6, a surgeon's control panel 7, a surgeon's wand 8 with an appliance/applicator 9, two appliance/applicator trays 10, and a master control panel 11. These equipment items are described briefly in the following paragraphs. It should be apparent that the small appliance/applicator 9 at the end of the surgeon's wand 8 is the key component of the entire system since it is the unit that physically accomplishes the anastomosis. The appliance/applicator 9 introduces the primary enabling technologies that permit the system to achieve the established design goals and will be described and discussed in detail herein.

The mobile transport cart 1 is preferably constructed with a stainless steel exterior for ease in maintaining cleanliness to hospital standards. The unit is equipped with large wheels on the front and swivel casters on the rear and is easily moved around the hospital manually by one person grasping the transport handle 12. The cart is also equipped with stabilizer pads 13, which extend on command to hold the cart in place during a surgical procedure and may be electrically or mechanically operated.

The cart 1 is equipped with a master control panel 11 that provides all power on/off functions, mast positioning, self-test diagnostics and equipment status displays. The master control panel 11 is equipped with a master lock feature that allows only authorized users access to the use of the SAS system. The panel 11 has visuals that provide feedback on the state of the operational steps and the available appliance/applicators 9 in the trays 10. The cart 1 is equipped with a heavy-duty, permanent electrical power cable which is “green dot” coded UL Hospital Grade for safety in the operating room environment.

Within the SAS cart 1 is an electrical power supply and backup system compatible with hospital alternating current power. The power backup system will provide power to the SAS in the event that the hospital power supply fails. The power system is located in the lower compartment of the cart 1 because of its relatively large size and weight. The weight is used advantageously to improve the stability of the cart 1 during a surgical procedure or during transport. Also in the lower portion of the cart 1 is the power conditioning/distribution unit which provides regulated power for the various functions of the SAS unit. These requirements range from the high power devices such as mast drive motors and stabilizer drive motors to low voltage/low power devices for the computer, the control panels/displays, and the instrumentation/status reporting circuitry.

The cart 1 is configured with permanent electrical harnesses, with special attention to routing, grounding and shielding so that power and signal circuits are properly separated, isolated and interference-free. The cart 1 also houses computational units with digital processor/memory devices to provide computational capabilities equivalent to those of the latest state-of-the-art, high-performance personal computers or workstations. The computational capabilities are initially sized to provide a 100% performance margin to assure adequate allowances for software enhancements and additional system functions that may be deemed desirable for future performance improvement. The cart 1 preferably houses two circuit card assembly (CCA) cages with six CCAs per cage; spare CCA slots are available to allow a future growth in functions without redesign of the basic unit. The cart 1 also houses monitoring instrumentation and recorders.

The cart 1 houses a mast assembly 2 drive system which, on command, extends the top mast 14, main mast 15 and the appliance/applicator kit utility tray 10 to the desired height. The vertical drive system preferably consists of an electric motor with speed reducer coupled to a gear that engages a rack on the vertical mast although other suitable electromechanical or hydraulic systems affording a similar degree of reliability and control may be used. The high gear ratio and a brake help achieve the desired resistance to mast movement once the mast is in the desired position. The mast position is controlled normally from the master control panel 11 on the cart 1, but it also can be controlled from the surgeon's control panel 7.

An appliance/applicator kit 16 comprises a sterile appliance/applicator 9 itself contained in a sealed sterile container with certain important attributes. First, the appliance/applicator 9 is restrained in a fixed position within the container, yet it can be easily removed after the seal has been broken and the lid removed from the container. Second, a plastic pinion projecting into the transmission portion of the appliance/applicator 9 holds the appliance/applicator's input gears in the proper initial position during shipment and storage. This restraint pinion is manually removed after the container's lid has been removed. Third, the container has a set of bar code symbols on its bottom and on its seal that indicate the type and size of appliance/applicator 9 contained within. Fourth, the removal of the appliance/applicator 9 from the container also causes the removal of a special bar code symbol on the bottom of the container which indicates that the appliance/applicator 9 has been used or is otherwise unavailable for use. The reason for these features of the appliance/applicator kits 16 will become apparent later.

In order to maintain and display the status of the appliance/applicator kits 16 (type, size, ready/used) in the appliance/applicator utility trays 10, the trays are equipped with a bar code scanner 17 that reads the bar code symbols on the kits 16 in the trays 10. This status information is available on the master control panel 11 display. The status database can be queried from and displayed on the surgeon's control panel 7. As the used appliance/applicator kits 16 are returned to a tray 10, the bar code scanner 17 collects data allowing the computer system to maintain accurate status on the number, type, and size of the used kits 16 and the available kits 16. The appliance/applicator utility trays 10 are also outfitted with light visuals (e.g. light emitting diodes). The role of the visuals is to indicate the correct appliance/applicator kit 16 in the tray once the surgeon, through voice commands or key pad entries, specifies the size and type of appliance/applicator kit 16 desired.

Mast Assembly

The mast assembly 2 is a two-part telescoping mast consisting of the top mast 14 and the main mast 15. In the transport configuration, shown in FIGS. 10A and 10B, the mast assembly 2 is in the lowered or stowed position as shown on the left in the figure. In the operational configuration, shown in FIG. 10C, the mast assembly 2 is activated from the master control panel 11.

When the main mast 15 rises, it lifts the appliance/applicator kit trays 10 and the bar code scanner 17 to the proper height for the anastomosis procedures. The top mast 14 then extends well above the top of the main mast 15 and raises the positioning arm 3 and all the subsequent components such as the surgeon's control panel 7, the working arm 5 and the surgeon's wand 8 into an initial position above the operating table.

The top 14 and main 15 masts are preferably constructed of composite material with a cylindrical cross-section to obtain the stiffness, light weight, and high strength needed to minimize flexure, which could cause undesirable motion of the surgeon's wand 8 during anastomosis procedures. Other materials that meet these requirements may be used. There are provisions for quick replacement of the appliance/applicator kit trays 10 while maintaining accurate alignment of the tray compartments with the bar code scanner 17. The top mast 14 has a smaller diameter than the main mast 15 that allows for a telescoping type motion. The electrical power, control, and instrumentation cables are routed through the center of the masts 14 and 15. The electrical harness in the masts is designed to accommodate the required mast rotation. A take-up reel in the cart allows the electrical harness to extend as the masts are raised and to retract as the masts are lowered.

The positioning arm 3 is designed to rotate approximately 350 degrees from the stowed position to provide a wide range of reachable positions for the other mechanisms and the surgeon's wand 8.

Positioning Arm

The positioning arm 3 is supported on the top mast 14 and manually rotated and locked at the desired position. The positioning arm 3 and its manually locked sliding segment 4 place the working arm 5 over the surgery site, and provide for the positioning of the surgeon's control panel 9 at an appropriate location. Manually operated locks 75 maintain the positioning arm 3 in a fixed position throughout a given anastomosis procedure.

Like the mast assembly 2, the positioning arm 3 is constructed of tubular composite or other appropriate material characterized as lightweight (for manual positioning of the arm and for reducing the power requirements and structural characteristics of the masts) and high stiffness and rigidity to properly sustain the load of the working arm 5 and surgeon's wand 8. The tubular structure provides electrical power and sensory pathways to the subsequent components such as the working arm 5 and the surgeon's wand 8.

The sliding segment 4 which is constructed of the same material as the positioning arm 3 but with smaller cross-sectional size to provide for telescoping action. The telescoping action provides increased reach for the working arm 5. The sliding segment 4 is manually positioned and locked in place during the preparation of the surgical site. The tubular structure provides electrical power and sensory pathways to the subsequent components such as the working arm 5 and the surgeon's wand 8.

Surgeon's Control Panel

The SAS is equipped with two surgeon's control panels 7, one mounted on each side of the positioning arm 3. Each surgeon's control panel 7 has an identical set of controls and displays. This allows operation and/or observation of the functions and status by the surgeon as well as by any other member of the surgical team at the direction of the surgeon. The dual arrangement provides more options on the position of the surgeon relative to the SAS unit and the operating table.

The surgeon's control panel 7 provides the interface between the surgeon and the master control panel 11 for all available functions. It can be used for a number of functions including interrogation of the SAS regarding identification and status of available and used appliance/applicator kits 16. During each anastomosis procedure, the status of the appliance/applicator 9 individual functions and sequences are displayed as the anastomosis procedure progresses to completion. During the anastomosis procedure, the surgeon will be occupied preparing the blood vessels and operating the surgeon's wand 8. It is expected that selected critical states in the operation of the appliance/applicator 9 will be monitored and displayed throughout the anastomosis process.

Each surgeon's control panel 7 is equipped with a power switch, a number of command buttons and status indicator lights, a microphone for voice activation, and a high definition display screen approximately 6 inches by 6 inches in size. Alternatively, the surgeon's control panel 7 may comprise a touch screen providing both data display and touch controls that change depending on the type of anastomosis being performed, the appliance/applicator 9 being used or the stage of the procedure.

Working Arm

The working arm 5 is a two-member articulated arm assembly that connects the sliding segment 4 of the positioning arm 3 to the surgeon's wand 8. The arm members are constructed of lightweight, high-stiffness material with close tolerance pivot joints. The mechanism is designed to hold a fixed position until the surgeon activates an “ACTION” button 18 on the surgeon's wand 8 allowing the surgeon to move the wand 8 and working arm mechanism to a new position. When the surgeon releases the “ACTION” button 18, the arm mechanism control devices (brakes) 32 hold the working arm 5 and the wand 8 rigidly in the new position. This action is somewhat analogous to the “click and drag” action of a computer mouse. This feature allows the surgeon to place the wand 8 in a desired location and orientation and then release it. Now, both hands are free to prepare for the anastomosis procedure or to perform other actions; the wand 8 will maintain its position until the surgeon holds down the “ACTION” button 18 again and moves the wand 8.

The fixed radius segment 20 of the working arm 5 is connected to the sliding segment 4 of the positioning arm 3 with a two-degree of freedom rotating joint 19. This rotating joint 19 is equipped with brakes 32 as described above and counterweights that balance the working arm 5 and the wand 8 about the two axes of rotation. This fixed radius segment is constructed of lightweight, high strength and high rigidity tubular composite or similar material and provides pathways for the IR T/R link as well as electrical lines (power and sensor). This segment houses the counterbalance mechanism, shown in FIG. 19, that automatically and dynamically balances the working arm 5 and the surgeon's wand 8 as the sliding segment 6 extends or retracts as the surgeon positions the A/A 9 while holding down the “ACTION” button 18.

The dynamic balancing provides for smaller size actuators and brakes thus allowing the surgeon to manipulate the wand 8 (and the working arm 5) freely with very little effort. The dynamic balancing principle will be discussed later.

The sliding segment 6 of the working arm 5 telescopes with the fixed radius segment 20 thus providing another translational degree of freedom. The freedom of the sliding segment 6 to translate is controlled by another brake 32 located at the end of the fixed radius segment 20. Like the other parts of the mast assembly 2 positioning arm 3 and working arm 5, this sliding segment 6 is constructed of lightweight and high strength and rigidity tubular composite or other suitable material. The distal end of the sliding segment 6 is a specially designed mechanism comprised of a two degree of freedom rotational device 21 equipped with brakes 32 and a special receptacle 22 that provides an additional degree of rotational freedom. The three degrees of freedom are equipped with brakes 32. This special receptacle 22 is an integral component of the working arm 5 and provides quick connect/disconnect features for ease of attaching and removing the surgeon's wand 8. In addition, a counterweight 23 is attached at the end of the receptacle 22 to balance the weight of the surgeon's wand 8.

The joints are outfitted with infrared communication capabilities for bidirectional communication between the surgeon's wand 8, the control panels 7 and the master controller 11. The IR transmitters and receivers are attached or housed in the joint 19 and rotational device 21 and receptacle 22.

As previously mentioned, each joint of the working arm 5 is outfitted with brake mechanisms 32 that lock the joint unless the surgeon is depressing the “ACTION” button 18 on his wand. The locking or freezing of the arm 5 in its current position allows the surgeon to use both hands for other actions and also holds the appliance/applicator 9 in a fixed position during the computer-controlled anastomosis sequences. The operation of the working arm 5 will be described in more detail later.

The arm mechanism control devices (brakes) 32 in arms 4 and 5 and the surgeon's wand 8 are preferably electrically operated friction brakes or clutch mechanisms which release when the “ACTION” button 18 is pressed and engage when the button 18 is released. For example, electronic solenoid operated clutch mechanisms at the joints would provide suitable brakes where pressing and holding the “ACTION” button 18 energizes the solenoids to release the clutch mechanisms thereby permitting free motion and positioning of the wand 8 by the surgeon. Upon release of the “ACTION” button 18, the solenoids are de-energized allowing the clutch mechanisms to return to their engaged position thereby locking the working arm 5 and the wand 8 in the desired position. With this arrangement braking of the working arm 5 and the wand 8 is a purely mechanical operation and electrical power is required only to energize the solenoids to release the clutch mechanisms thereby minimizing the electrical requirements of the SAS. A solenoid operated clutch mechanism as described here is only one example of an arm mechanism control device suitable for use in the present invention. Other electromechanical brake systems may be used without departing from the scope of the invention.

Surgeon's Wand

The surgeon's wand 8 is a multifunction electromechanical device with built-in sensors, actuators, electronics, and power storage. The surgeon's wand 8 attaches to the special receptacle 22 at the end of the sliding segment 6 of the working arm 5. The wand 8 will be described in considerable detail later.

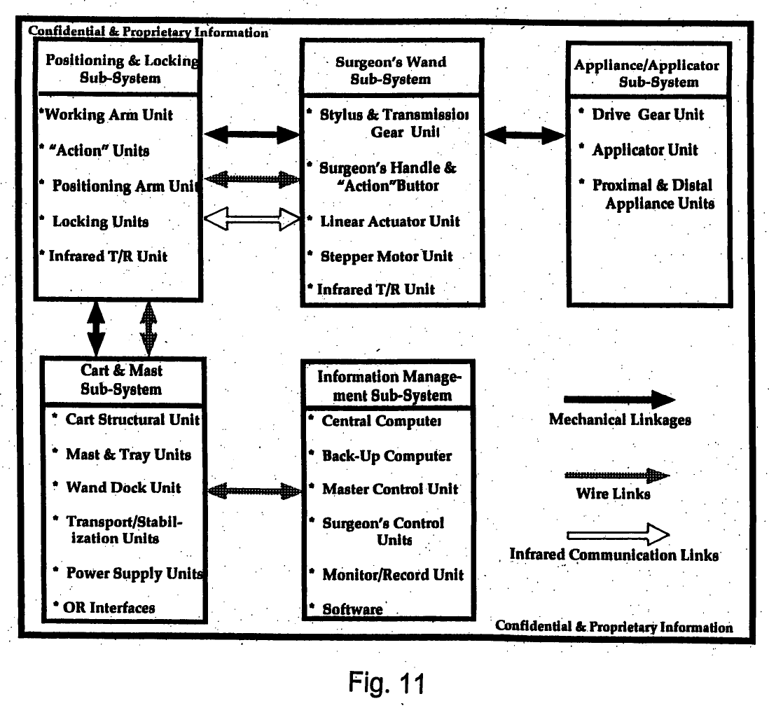

The SAS is comprised of five major subsystems: cart and mast sub-system, positioning and locking sub-system, surgeon's wand sub-system, appliance/applicator sub-system and the information management sub-system. These subsystems and their interdependency are illustrated and summarized in the functional block diagram in FIG. 11.

The major mechanical and communication connections between individual subsystems are indicated by the coded arrows signifying either one-way or two-way paths. Mechanical functions are manual, electrically powered via switches/buttons, or voice-activated/electrically powered. Many voice or switch-activated functions are automatically sequenced via computer and robotic control systems. Key automatic and electrically-powered functions are configured to allow manual actuation as backup where practical. Communication paths use electrical wiring or infrared (IR) transmitter/receiver (T/R) devices. Optical communications over optical fibers may also be used. The critical communication paths that utilize IR T/R links are those portions of the working arm where the friction, weight and volume associated with electrical wiring would be extremely undesirable.

At this point, it is desirable to summarize the essential features and operation of the SAS concept. This summary should help focus the subsequent detailed discussions of the surgeon's wand 8, the working arm 5 and the appliance/applicators 9. Some of these features are:

The ends of the blood vessels to be anastomosed are stretched to the desired shape with specially designed balloons inserted into the lumen and inflated with a saline solution. The extensive use of Fogarty balloons in angioplasty procedures has shown that blood vessels can be stretched by this method without imparting significant damage to the intima.

After the ends of the blood vessels have been stretched to the desired shape, they are held in that shape and positioned by external precision grippers. These grippers utilize microscopic stainless steel tines to partially penetrate the external fibrous tissue and smooth muscle layers of the blood vessel without reaching the endothelium.

The grippers are fastened together so the gripped blood vessel ends are placed in apposition to form a hemodynamic seal.

Attaching, stretching, gripping, positioning, fastening, etc. are accomplished sequentially with accuracy and precision by micro-electro-mechanical systems (MEMS) or other suitable systems.

The MEMS are, in turn, operated by a pre-programmed computer so that the anastomosis is accomplished accurately and repeatably in a few seconds.

Although the process is highly automated, the surgeon is in complete control at all times and can visually inspect the completed anastomosis as well as override the automatic sequences.

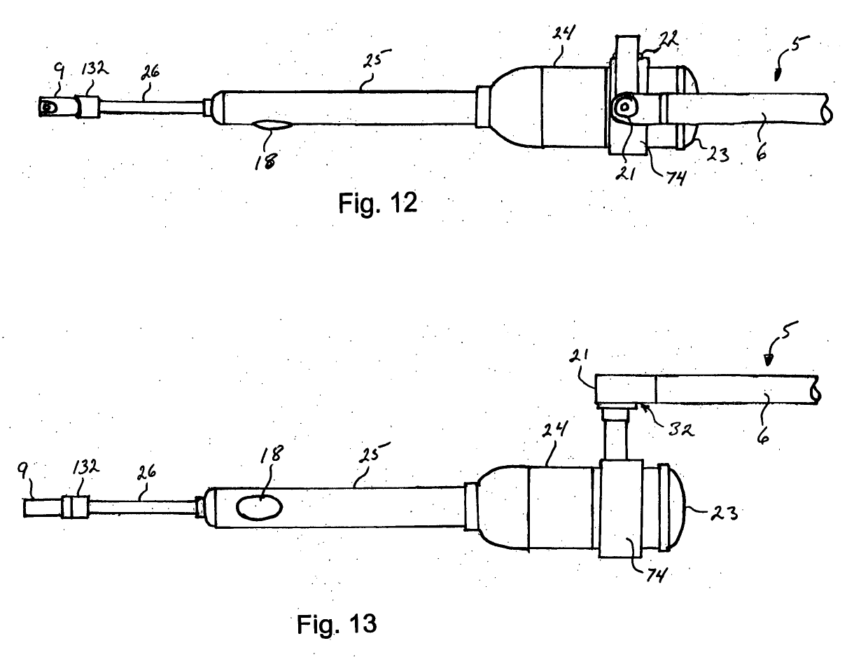

FIGS. 12 and 13 illustrate the surgeon's wand 8. FIG. 12 is a top view and FIG. 13 is a side view.

The main components of the surgeon's wand 8 are the wand functional unit 24, the surgeon's handle 25, and the stylus 26. The surgeon's wand 8 is approximately six inches long excluding the stylus 26. The wand 8 is manipulated by the surgeon, hence its size, shape and weight distribution are designed to be ergonomic for optimal comfort and function during the surgery. As will be explained later, the working arm 5/surgeon's wand 8 combination is equipped with low-friction bearings and counterbalances so that the surgeon's handle 25 can have six degrees of freedom, and the surgeon will feel essentially no weight or friction when he moves the handle 25. The wand functional unit 24 is attached to the working arm 5 through a quick connect/disconnect joint at the receptacle 22, which facilitates the easy exchange of wands 8 if desired. Receptacle 22 is part of an annular ring 74 into which the wand functional unit 24 fits and which provides rotation of the wand 8 around its longitudinal axis. As with the joints of working arm 5, annular ring 74 includes bearing means and a brake 32.

The wand functional unit 24 contains the two actuators 27, the transmission device 28, the electronics/power supply 29 and the infrared transmitter/receiver units 30. The wand functional unit 24 also contains a counterweight 23 for balancing the wand 8 about the last rotational joint of the working arm 5 at receptacle 22. In addition, the brake 32 for this last joint is housed here.

The surgeon's handle 25 is connected to the wand functional unit 24. It houses a transmission mechanism consisting of a number of concentric tubular hollow shafts 31 that transfer power from the wand functional unit's actuators 27 through the stylus 26 and to the appliance/applicator 9. The four working arm joints with their associated bearings and brakes 32 and the special receptacle 22 and annular ring 74 give the handle 25 six degrees of freedom for positioning and orienting the appliance/applicator 9 for anastomosis as will be described later.

The surgeon's handle 25 is equipped with an “ACTION” button 18 that provides an interface between the surgeon and the computer to unfreeze the handle 25 and working arm 5 by disengaging the brakes 32 associated with the joints when the button 18 is depressed and to initiate the sequential anastomosis actions when the button 18 is double-clicked. The surgeon uses this handle 25 to position and orient the stylus 26 and appliance/applicator 9 as he desires.

The stylus 26 provides the interface between the handle 25 and the appliance/applicator 9. The stylus 26 is mainly a housing and provides power pass-through and interface with the appliance/applicator 9. The stylus 26 can be of different shapes and lengths for hard-to-reach areas. The end of the stylus 26 is a transmission mechanism that connects with the appliance/applicator 9 and provides the required rotational drive to actuate the various components of the appliance/applicator 9, as will be explained later.

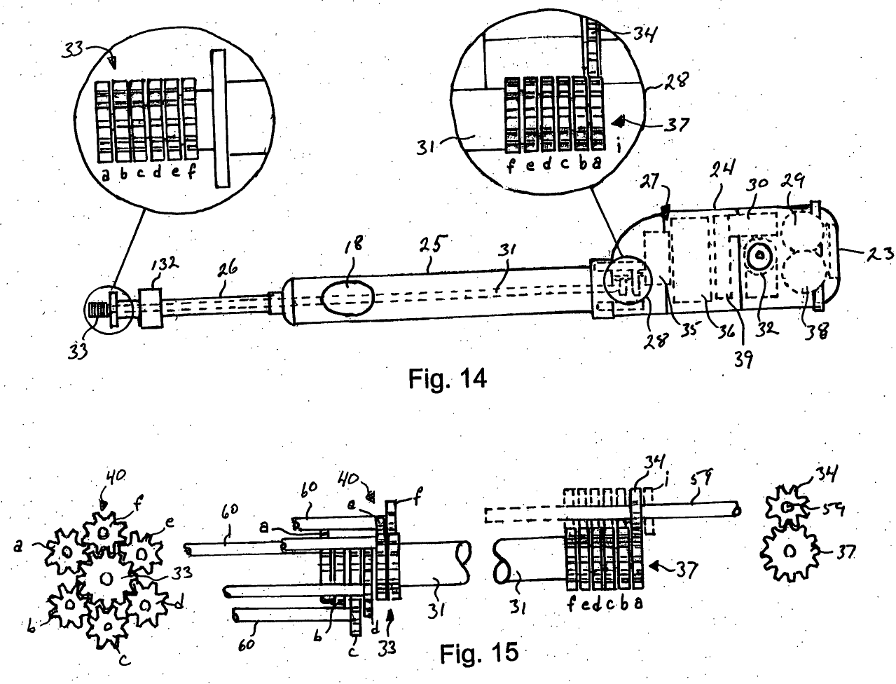

An example layout for the surgeon's wand 8 is shown in FIG. 14. The wand functional unit 24 contains a set of transmission gears 37 that provide power to the correct transmission shaft 31 through an innovative mechanism. Each gear 37 is mounted on the distal end of one of the concentric drive shafts 31. The applicator drive gears 33 are mirror images of the transmission gears 37 and are each mounted on the corresponding proximal end of the concentric drive shafts 31. The applicator drive gears 33 are at the end of the stylus 26 and provide the rotational drives to the appliance/applicator 9. There are two actuators 27; namely, a linear actuator 35 and a rotational actuator 36. The linear actuator 35 is a seven-position actuator that positions the drive pinion 34, mounted on a seven position translating drive shaft 59, at the correct location for engaging the desired transmission gear 37. Once the drive pinion 34 is correctly positioned, the rotational actuator 36 (a stepper motor) starts and provides the precise amount of rotation to the correct transmission shaft 31 so that the appliance/applicator 9 will be actuated for that particular step in the anastomosis sequence. The positioning and rotation are predetermined based on the motion sequence of the appliance/applicator 9 during the anastomosis process. The actuators 27 are controlled by the computer through the control electronic cards 39 and obtain their power from the batteries 38 in the wand functional unit 24.

FIG. 15 illustrates the transmission assembly of the surgeon's wand 8. The transmission gears 37 are located in the wand functional unit 24 of the surgeon's wand 8. The drive pinion 34 selectively engages any of the six transmission gears 37 when the linear actuator 35 translates it to the proper position. There is a seventh position that the drive pinion 34 can be translated to where it does not engage any of the six transmission gears 37. This seventh position is the initial position and is labeled i. The six concentric shafts 31 transmit the power to the applicator drive gears 33, which, in turn, power the actuator drive gears 40. The actuator drive gears 40 are located in the A/A 9 where they are stacked in a planetary gear configuration. Each actuator drive gear 40 is connected to an actuator drive shaft 60 that is continuously engaged with a component in the A/A 9. The actuator drive gears 40 will be described later in more detail when the A/As 9 are discussed.

Note that a given shaft 60 in the A/A 9 is powered and rotates only when the drive pinion 34 is positioned and engaged with the corresponding transmission gear 37 and when the rotational actuator 36 turns in response to a command from the computer.

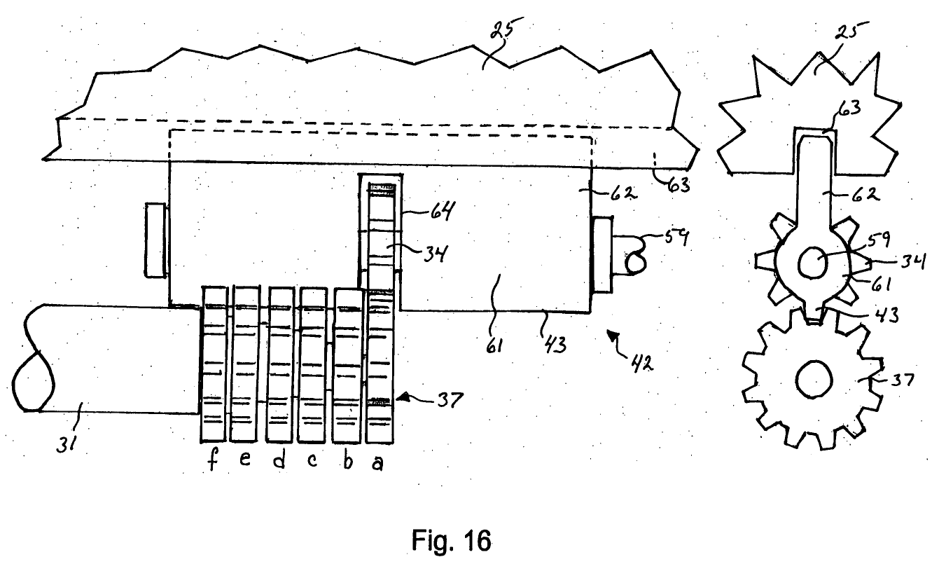

As shown in FIG. 16, transmission 28 in the wand functional unit 24 includes snubber assembly 42 which consists of a snubber body 61 located on the end of the seven position translating drive shaft 59. Snubber body 61 has a length that is substantially twice as long as the array of transmission gears 37 and is provided with a dorsal stabilizer blade 62 and a ventral snubber vane 43. Stabilizer blade 62 is slidable within a snubber stabilizer slot 63 located in the wall of the surgeon's handle immediately adjacent to the transmission 28. Snubber vane 43 is opposite to stabilizer blade 62 and substantially corresponds in shape and size to the teeth of drive pinion 34 so as to engage transmission gears 37. Drive pinion 34 is located in a gap 64 substantially midway along snubber body 61 such that drive pinion 34 is limited to engagement with only one of transmission gears 37 at any one time in response to linear positioning by the linear actuator 35. The linear actuator 35 positions the drive pinion 34 and Snubber assembly 42 based on signals from the control electronics which, in turn, are commanded by the computer. This positioning provides a positive locking mechanism to all but the transmission gear 37 corresponding to the stopped location. For example, as shown in FIG. 16, if transmission gear a is to be actuated, then the drive pinion 34 and snubber assembly 42 are moved to position a by the linear actuator 35 where the pinion 34 is engaged with transmission gear a. At the same time, the snubber vane 43 locks the other gears 37 (b, c, d, e, f) and does not allow them to rotate. Then, the rotational actuator 36 is actuated and rotates the drive pinion 34 which, in turn, drives transmission gear a that rotates the corresponding shaft 31 to ultimately rotate actuator drive gear 40 a and its corresponding shaft 41 a in the applicator 9.

The snubber 42 at position i provides a positive lock to all the transmission gears 37. The driving mechanism should be at this initial position whenever a new A/A 9 is initially attached to the stylus. This initial location will guarantee that all the subsequent drive motions will be performed with the proper relative relationship. The gear teeth on the pinion 34 are designed to provide for easy engagement when the pinion 34 is repositioned.

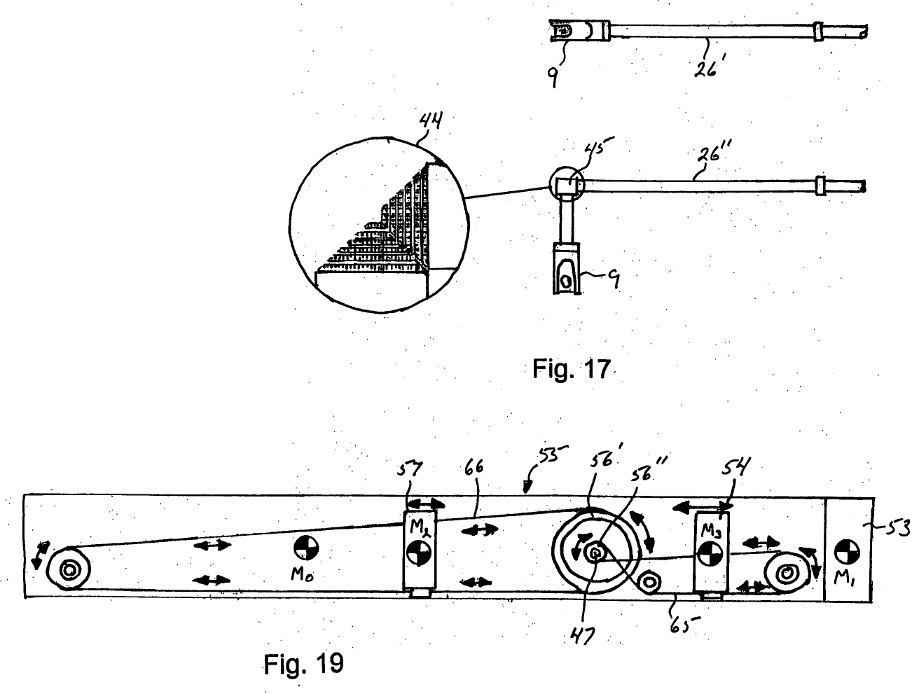

The surgeon's wand stylus 26, as already mentioned, can have different lengths and/or geometric configurations to provide the surgeon with more flexibility in reaching the surgery site. There are two main types of styli 26, shown in FIG. 17: straight 26′ and right angle 26″. The functionality remains the same, i.e. it provides a pathway for transferring the power from the transmission gears 37 to the A/A 9. The end of the stylus 26 is a concentric gearing arrangement connected to the six shafts 31 that transfer power from the transmission gear 37 to the A/A 9. The right angle styli 26″ provide for power transfer through a concentric bevel gear 44 arrangement housed inside the 90-degree elbow joint 45.

As previously discussed, the working arm 5 has two primary functions, viz., it provides a secure communication path between the surgeon's wand 8 and the positioning arm 3 (and thus the computer) and it provides a means for rigidly maintaining the precise position of the surgeon's wand 8 when the “ACTION” button 18 is not depressed. These functions must be accomplished without introducing undue friction, loads or limitations that would degrade the surgeon's ability to freely move the wand 8 with precision and dexterity when the “ACTION” button 18 is depressed.

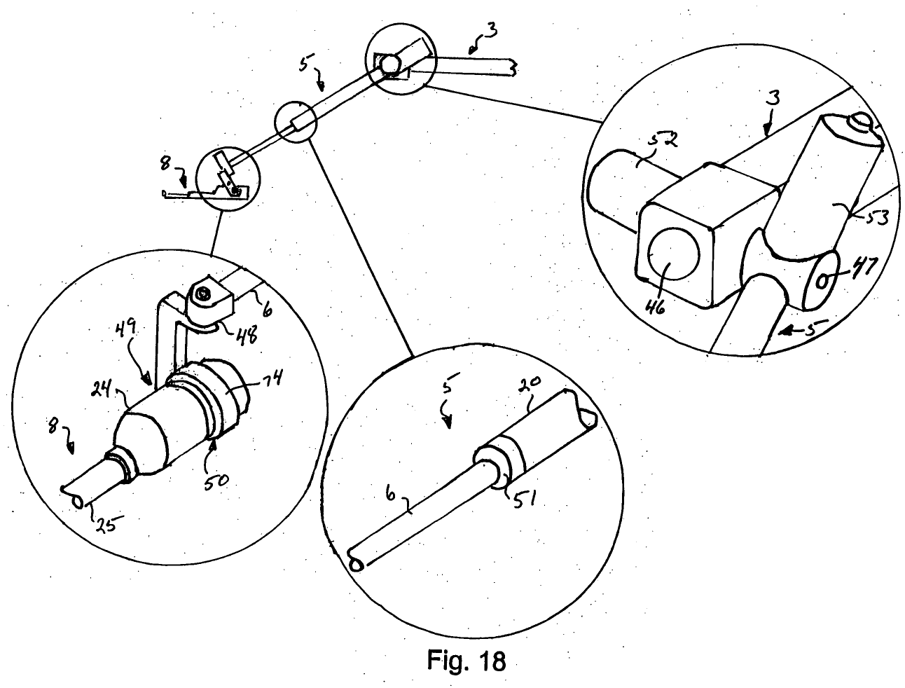

The desired six degrees of freedom for the surgeon's wand 8 can be achieved with six low-friction joints as indicated in FIG. 18. In this example arrangement, five rotating joints 46-50 and one sliding joint 51 have been chosen.

The connection of the working arm 5 to the positioning arm 3 is accomplished with rotating joints 46 and 47 to provide two degrees of freedom. Joint 46 provides for 360 degrees of low friction rotation about the axis of the fixed positioning arm 3. Joint 47 allows the fixed radius segment 20 of the working arm 5 to rotate about an axis that is perpendicular to the rotation axis of joint 46. However, joint 47 is offset as shown so that the working arm 5 can rotate about the joint 47 axis for almost 360 degrees, the exception being the small, unused angle where the wand 8 could interfere with the positioning arm 5. The offset necessitates the addition of a counterweight assembly 52 on the opposite side of the positioning arm 5 to prevent a rotation about joint 46 due to gravity.

Similarly, joint 47 requires a counterweight 53 opposite the working arm 5 to balance the effects of gravity. However, this counterweight 53 must provide for dynamic balancing since the length of the working arm 5 varies as the surgeon moves the wand. Actually, two counterweights are present: the first is a fixed counterweight 53 to balance the fixed radius segment 20 of the working arm 5 and the second is a counterweight 54 that moves in coordination with the sliding segment 6 of the working arm 5 to counterbalance the mass of the sliding segment 6 and the mass of the surgeon's wand 8. The principles governing the design of this dynamic balancing system 55 will be discussed later. To minimize volume requirements, all counterweights are made of a very dense material such as tungsten or the like.

Joint 51 is located at the end of the fixed radius segment 20 and is a low friction sliding joint that allows the sliding segment 6 to move linearly to vary the length of the working arm 5. This joint 51 also restrains the sliding segment 6 from rotating about its own axis. In this specific case, it is anticipated that the sliding segment 6 will be designed to move linearly approximately 30 centimeters. Consequently, the end of the working arm 5 has three degrees of positional freedom through the action of joints 46, 47 and 51. Thus, the end of the working arm 5 can be easily moved to any point within an imaginary spherical shell 30 centimeters thick that is centered at the end of the positioning arm 3 and has an outside radius equal to the fully extended length of the working arm 5. The only exception is the small, unused volume where there would be interference between the surgeon's wand 8 and the positioning arm 3.

The surgeon's wand 8 is attached through a rigid fitting to the end of the working arm 5; therefore, the wand 8 has the same three degrees of positional freedom as the end of the working arm 5. In addition, joints 48, 49 and 50 are low friction rotational joints that permit the surgeon's handle 25 to rotate about three mutually perpendicular axes that intersect at the center of gravity of the surgeon's wand 8 (including the stylus 26 and A/A 9). This arrangement allows the surgeon's handle 25 to be freely “pointed” in any reasonable direction. When the “ACTION” button 18 is depressed, the surgeon can now use his handle 25 to move the A/A 9 with three degrees of positional freedom and three degrees of directional freedom with the sensation that everything is kinematically frictionless and weightless. It should be noted that this working arm/wand arrangement should have many other useful applications.

The fact that the surgeon's wand 8 and the sliding segment 6 of the working arm 5 move linearly as much as, say, 30 centimeters relative to the fixed position of joint 47 requires that their combined weights M₂ be balanced by a counterweight M₃ 54 that moves in the opposite direction on the other side of the pivot point at joint 47. A conceptual layout of a balance system 55 of this nature is shown in FIG. 19 in which M₀ represents the center of mass and center of gravity location of the fixed radius segment 20 of the working arm 5 without fixed counterweight 53, represented here by M₁. M₂ represents the center of mass and center of gravity of the surgeon's wand 8 and the sliding segment 6 of the working arm 5 and M₃ represents the moving counterweight 54, having a mass that is N times the mass M₂ 57, the combined weight of wand 8 and sliding segment 6. The design problem is complicated by the fact that the moment arm of the surgeon's wand 8 and the sliding segment 6 of the working arm 5, represented by mass M₂ 57, is quite large and translates a considerable distance during use. To counterbalance this mass M₂ 57 with a counterweight of equal mass and moment arm would result in an undesirably long and awkward counterbalance mechanism.

The solution chosen here is to make the mass of the moving counterweight M₃ 54 N times larger and its moment arm 1/N times smaller to provide the balancing moment at the pivot point. Since the counterweight's 54 moment arm must vary as the working arm 5 is extended and retracted, the counterweight 54 must move 1/N times as far as the sliding segment 6 of the working arm 5. This is achieved through a pair of pulleys 56 that rotate together about the axis of joint 47. The larger pulley 56′ is belted to mass M₂ 57 by belt 66 so that it rotates as the sliding segment 6 is extended or retracted. Pulley 56″ rotates accordingly since it is rigidly attached to pulley 56′. Pulley 56″ is, in turn, belted to counterweight 54 M₃ with a crossed belt 65 so that counterweight 54 M₃ moves in the opposite direction when mass M₂ 57 is moved. The diameter of pulley 56′ is N times the diameter of pulley 56″ so that balance is maintained as mass M₂ 57 moves. The conditions for achieving balance in the system 55 are expressed by the formulas x₀/x₁=M₁/M₀ and x₂/x₃=N=M₃/M₂, where x₀ is the distance between joint 47 and M₀, x₁ is the distance between joint 47 and fixed counterweight 53, x₂ is the distance between joint 47 and M₂, and x₃ is the distance between joint 47 and M₃.

By choosing pulleys 56 so that N is as large as practicable, say N=6, and by choosing a very dense material for the counterweights 54 and 53, say tungsten, it is possible to design a balance system 55 that is acceptably compact. In preferred design, the pulleys and belts represented in FIG. 19 would be miniature sprockets and chains in order to minimize friction with the sprocket bearings of low friction material. Similarly, the movable counterweights 54 and 57 and the sliding segment 6 would be supported on low friction bearing members such as rollers, wheels, sliders or the like.

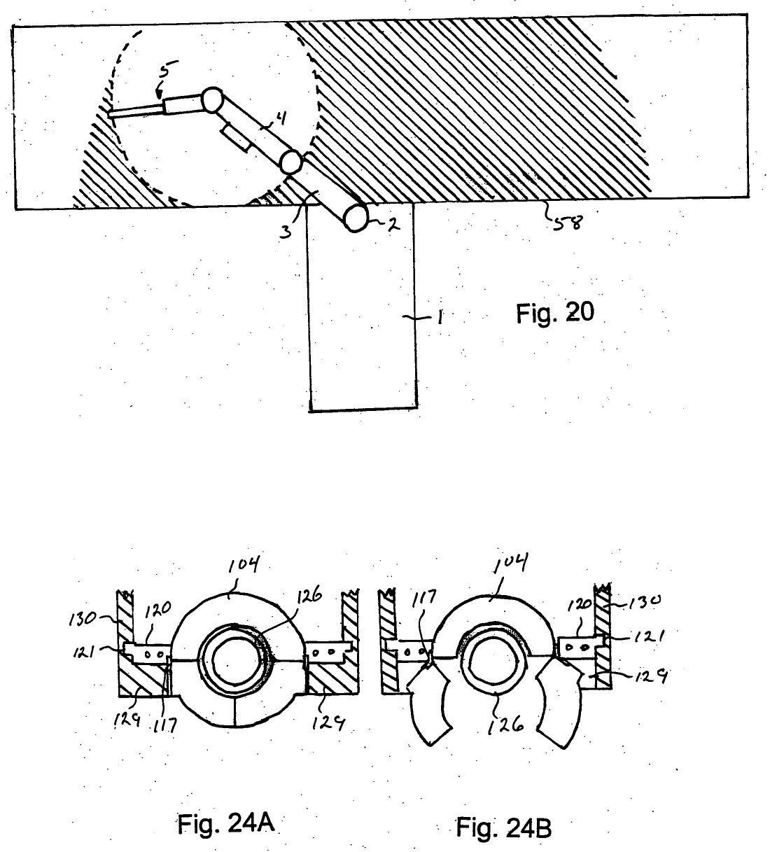

FIG. 20 shows an example of the working arm 5 footprint for a particular SAS cart location in which the SAS Cart 1 is located adjacent to the operating table 58 at a location chosen in advance by the surgeon to minimize potential interference with other activities occurring around the table 58. The surgical staff positions and stabilizes the cart 1 and then raises the mast system 2 to convenient working heights. The positioning arm 3 is then positioned and locked so that the tip of the stylus 26 easily reaches the surgery site with a generous margin of error. The footprint shown in FIG. 20 is for the tip of the working arm 5 at the surface of the table 58 with the positioning arm 3 approximately 50 centimeters above the table surface. The size of the footprint can be roughly scaled by recalling that the width of the cart 1 is approximately 30 centimeters.

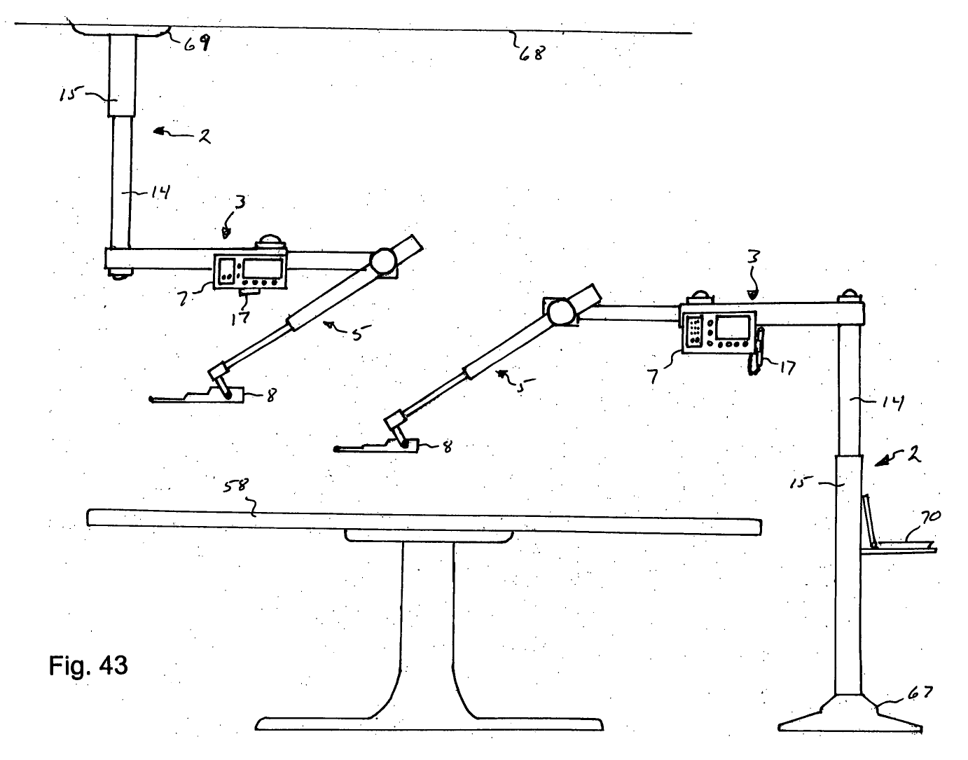

In an alternative embodiment, shown in FIG. 43, the mobile cart 1 may be eliminated. In this embodiment, suitable for portability and field use, the mast system 2 is provided with a floor stand 67 or a means to secure it to an operating table or other structure in an operating room and the telescoping top mast 14 and main mast 15 may be operated by simple mechanical means. As a further alternative, the mast system 2 may be attached to the ceiling 68 of the operating room so as to be suspended over the operating table 58. In addition, a ceiling suspended assembly may include a track mechanism 69 providing additional flexibility whereby the arm assembly may be moved in a linear manner relative to the operating table 58.

In such alternatives, the structure and function of the mast system 2, positioning arm 3, working arm 5 and surgeon's wand 8 are the same as previously described. The differences are directed to the power source, computer control systems and appliance/applicator storage and inventory systems. In the case of electrical power, appropriate provision may be made to draw filtered electrical power from the hospital main power source, a generator, or other power source. In addition, particularly in the case of the free standing or table attached model, since the actual power requirements are low, a portable and/or back-up power supply may be provided.

Alternative computer control is provided through a portable computer 70, such as a laptop or tablet PC, or even a hand held computer communicating with the mast 2, arms 3, 5, surgeon's wand 8 and surgeon's control panel 7 by infra-red, wireless or wired connection, such as USB or fire wire connectors, or such other communication means as are common in computer systems. Alternatively, the computer control system may be incorporated as an integral part of the surgeon's control panel 7 in which case a multifunction touch panel display screen such as is common on hand held and tablet PC units is preferred to provide both data read-out as well as functional control of all aspects of the apparatus and method of use. The portable computer 70 is preferably pre-programmed with the commands and routines to operate the mast 2 and arms 3, 5, the surgeon's wand 8 and the appliance/applicator 9 operations based on the type of anastomosis to be performed and the appliance/applicator 9 being used. A separate or built-in bar code scanner 17 is provided to read the code from the appliance/applicator kit 16 thereby identifying the particular anastomosis routine to be activated by the computer 70. Data concerning the appliance/applicator 9 used is correlated with the hospital inventory or a separate inventory data base programmed or entered into the memory of the portable computer 70. In the portable model, since there is no provision for storage of trays holding a plurality of appliance/applicator kits 16, inventory of appliance/applicator kits 16 in the form of cases are preferably shipped with the SAS with each case having a code, disk or other means providing data to identify the type and number of each appliance/applicator kit 16 packed therein. Entry of this code, disk or other means into the portable computer 70 provides the inventory data base for that particular case and correlates the case with the operational parameters of the SAS.

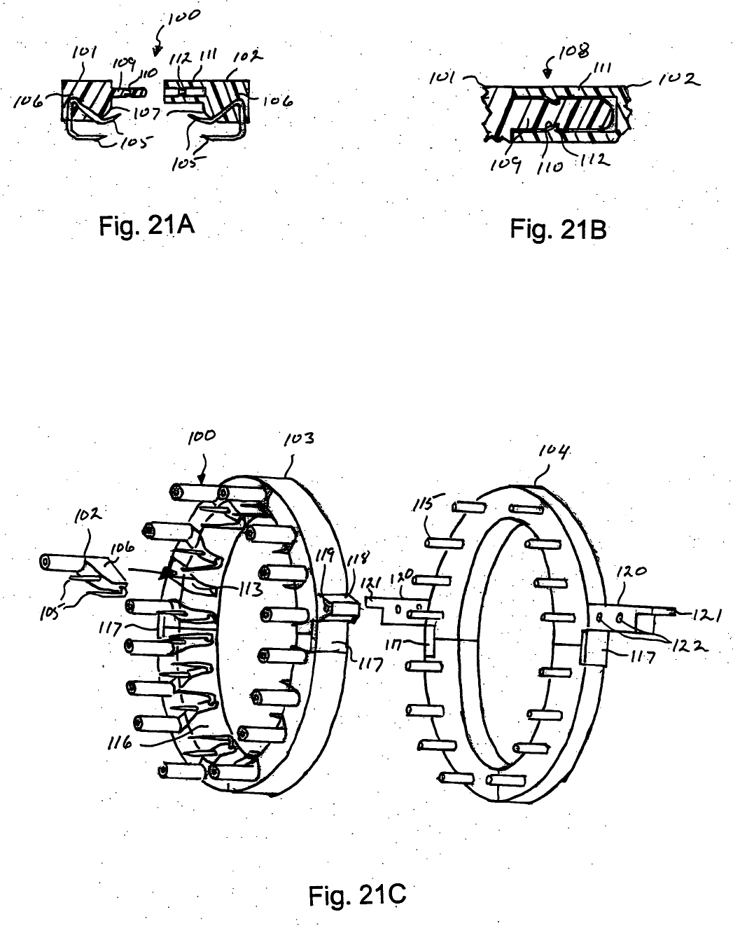

Turning now to the appliance/applicator assemblies; the A/A for a Type I anastomosis will be described first followed by a description of a Type III anastomosis and appliance/applicator therefor. It is not necessary to describe a Type II A/A since the side graft portion of a Type III A/A is one-half of a Type II A/A and it is easy to see how a Type II would be designed. As noted previously, the components of the appliance/applicator assemblies that remain in the body at the conclusion of an anastomosis procedure are the precision anastomosis grippers, or PAGs. FIGS. 21A-C illustrate a Type I PAG 100 and PAG assembly in accordance with the present invention.

The main components comprising a Type I PAG assembly are the proximal 101 and distal 102 PAGs, two PAG holders 103 and two PAG ejectors 104. Only the distal PAG holder 103 and PAG ejector 104 are shown in FIG. 21C since the proximal holder and ejector would be identical to the distal holder and ejector (but equipped with proximal 101 PAGs). Each of these components has certain characteristics and properties that provide functionality for the anastomosis process.

Each PAG 100 consists of two stainless steel tines 105 projecting from a specially shaped hard plastic body 106 as indicated in FIG. 21A. Typically, the diameter of the tines 105 is about one-sixth to one-third of the vessel wall 128 thickness. The tines 105 are sharpened to provide easy penetration of the outer layers of the blood vessel 126 at two locations. The sharpened tips of the tines 105 are positioned approximately one-half of the blood vessel wall thickness away from the hard plastic body 106 holding the tine 105. This critical location of the tine tips relative to their plastic bodies prevents complete penetration of the blood vessel wall so that the intima will not be disturbed during the anastomosis process. The hard plastic body 106 has a curved portion 107 that defines the radius of curvature that will be used when the subject blood vessel 126 is partially everted. This radius of curvature depends upon the specific design requirements but is usually roughly equal to the blood vessel wall 128 thickness. The plastic body 106 must be made of a relatively hard material that has long term compatibility with living tissue (such as polypropylene, for example) since PAGs 100 constitute the appliance that will be left inside the patient's body.

As previously mentioned, there are two types of PAGs 100, the proximal 101 and distal 102. These are similar in that they hold identical tines 105 and have identically shaped main bodies 106; but their connecting structures 108 are quite different. The connecting structures 108 are preferably male and female cooperating elements with a cooperating detent means to prevent inadvertent separation of the proximal 101 and distal 102 PAGs after implantation. Preferably, the connecting structure 108 of the proximal PAG 101 is the male element and comprises a solid rod 109 with an end that is chamfered to provide for easy insertion into the corresponding female element of the distal 102 PAG's connector. As a detent the rod 109 also has a step-shaped groove 110 around its perimeter at a predetermined distance from the PAG's main body 106.

The distal PAG 102 has a female element comprising a hollow tube 111 connecting structure 108. The proximal 101 and distal 102 PAGs mate to provide a positive locking mechanism by inserting the solid rod 109 into the tube 111. The inner diameter of the tube 111 structure should be a little larger than the outside diameter of the solid rod 109 of the proximal 101 PAG, and the length of the tube 111 should be a little longer than the solid rod 109. In addition, the tube 111 has a step 112 that is a single ridge around its inside perimeter at a predetermined distance from the PAG's main body 106. The positive lock is achieved when the groove 110 on the solid rod 109 passes by the step 112. This is clearly shown in FIG. 21B which is a cross section of the connecting structure 108. There are many different approaches for accomplishing a positive lock, and the configuration shown in FIG. 21A is only one example.

The connector 108 also performs another vital function. It not only rigidly connects the proximal 101 and distal 102 PAGs, but it also sets the separation distance between the two PAGs. This separation distance should be somewhat less than the total thickness of the two vessel walls when they are placed in apposition for joining. This distance should be small enough to assure that the two partially everted vessel ends are firmly pressed together to achieve a hemodynamic seal but large enough to avoid damaging the vessel tissue. This will become apparent as the actual anatomosis process is described.

The PAG holder 103 is a specially shaped annular ring whose inside diameter is large enough to allow the subject blood vessel 126 to pass through it. The PAG holder 103 has a series of notches 113 shaped so that PAGs 100 can be pressed into the holder 103 with a slight interference fit so that the PAGs 100 will be held firmly in place as shown in FIG. 21C. These notches 113 extend through approximately two-thirds of the axial thickness of the holder 103 so that the holder 103 still has structural integrity when no PAGs 100 are in place. At the base of each notch 113 is an axial ejector hole 114 that extends the rest of the way through the holder 103 to provide a pathway for the ejector pin 115 that will be discussed below.

The inside lip 116 of the holder 103 is rounded with the same radius of curvature as the curved portion 107 of the PAGs 100. When the PAGs 100 are in place, the holder/PAG combination presents a smooth, properly curved surface with projecting tines 105 as shown in the figure. The PAG holder 103 is made in three pieces and has two hinges 117 that allow the holder 103 to open for easy removal once anastomosis is completed. The holder 103 is also outfitted with two projections 118 with threaded holes 119 that allow a lead screw to axially translate the holder 103 relative to the PAG ejector 104 assembly as will be explained later.

The PAG ejector 104 is the same for both proximal 101 and distal 102 PAGs. The PAG ejector 104 is an annular molded component of the same diameter as the PAG holder 103. It has a number of ejector pins 115 molded into it at the same relative locations as the ejector holes 114 at the base of the PAG notches 113 in the holder 103. The diameter of the ejector pins 115 is very slightly larger than the ejector holes 114 described above. The PAG holder 103 is pressed onto the ejector pins 115 until each pin 115 is almost touching the base of its corresponding PAG 100. The ejector pins 115 are long enough to assure that they will push the PAGs 100 out of the PAG holder 103 when the ejector lead screw 155 translates the holder 103 toward the ejector 104. The PAG ejector 104 is also made in three pieces with two hinges 117 as shown. The ejector 104 is also outfitted with two projections 120 with tongues 121 that engage horizontal slots 131 in the A/A frame 130 so that the whole PAG holder/ejector assembly 156 can be translated axially by a translation lead screw. There are two holes 122 in the projections 120. One is a through hole for the ejector lead screw 155, and the second hole is threaded for the translation lead screw.

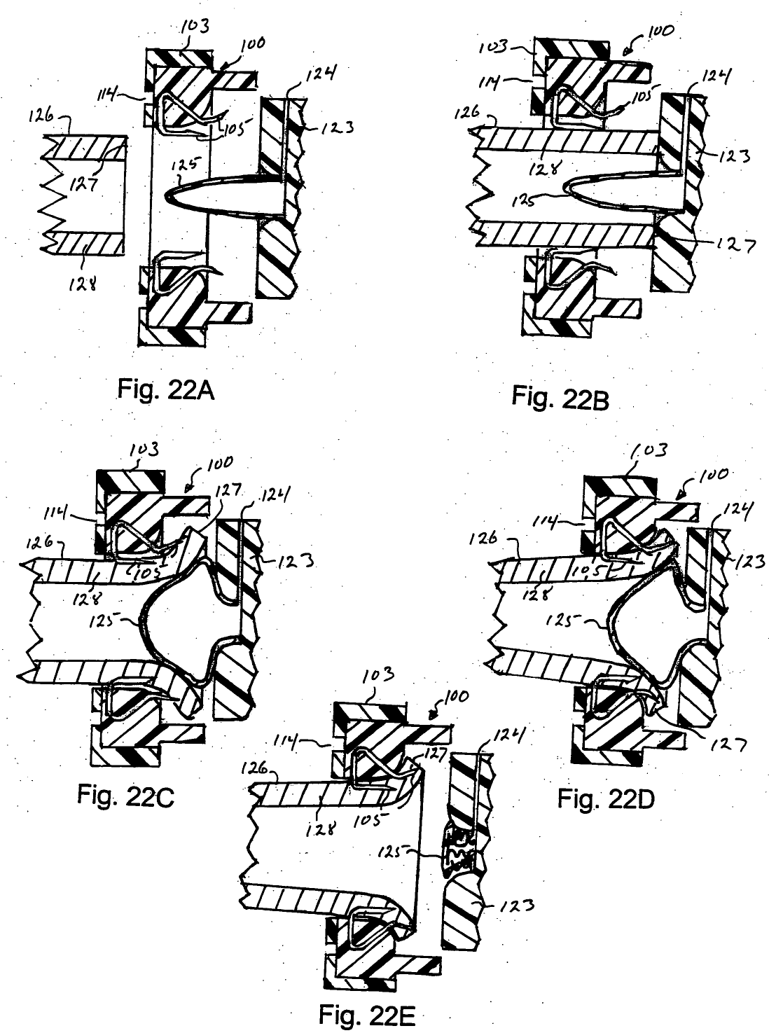

The following steps describe the Type I anastomosis procedure for a blood vessel and is correlated to FIGS. 22, 23 and 24. In this example, the distal end of a blood vessel 126, in this case a severed artery, was chosen for discussion. The process commences with the surgeon stopping the blood flow and then exposing and trimming the proximal and distal ends of the vessel 126 by conventional means. Next, the surgeon estimates the inside diameter and wall thickness of the vessel 126 and verbally requests that the computer indicate the location of the correct A/A kit 16 in the A/A tray 10. Either the surgeon or the technician manning the master control panel picks up the indicated A/A kit 16, breaks the seal 314, opens the lid 301 and removes the restraint pin 308. The surgeon inserts the drive gear 37 assembly at the tip of his wand stylus 26 into the A/A 9 and manually tightens the attachment screw cap 132. He then withdraws the A/A 9 from the container 300 and places the empty container 300 on the utility tray 10. At this point, everything is ready for the actual anastomosis process to begin.

As previously mentioned a key and novel aspect of the SAS concept is the use of intralumenal balloons to expand and partially evert the edges of the vessel to be anastomosed. The extensive use of Fogarty-type balloons in angioplasty procedures has demonstrated conclusively that properly used intralumenal balloons can stretch and deform blood vessels without significant damage to the intima. In the present case, each A/A 9 has two specially shaped balloons 125 (one proximal and one distal). The balloons 125 are mounted on a disk-shaped plastic component that is referred to as “the lollipop” 123. The lollipop 123 has internal passages 124 that permit each balloon 125 to be inflated with a saline solution. The saline solution is supplied by a microsyringe 147 that is an integral part of the A/A 9. When the A/A 9 is sealed in its container, the syringe 147 is full and the balloons 125 are fully deflated (“withered”). The piston of the syringe 147 is driven by one of the six drive gears 33 on the end of the wand stylus 26 through a lead screw arrangement that will be described later.

The surgeon then depresses the “ACTION” button 18 on his wand 8 and positions the A/A 9 at the anastomosis site with the desired orientation. By releasing the “ACTION” button 18, the wand 8 will maintain this position until the button 18 is held down again. The anastomosis is now executed through the following sequential steps:

Step 0, FIG. 22A: By verbal request or by control panel 7 entry, the surgeon commands “READY DISTAL”. This command causes the computer to actuate the syringe 147; saline solution is injected into the distal balloon 125 to an extent that fills (but not inflates) the balloon 125 so that it is in a quasi-rigid state. The computer then responds “DISTAL READY” both verbally and by display on the control panels 7. The axial diameter of the balloon 125 is approximately one-third to one-half the inside diameter of the subject vessel 126. At this point the PAG holder/ejector assemblies 156 are disposed between the vessel 126 and the lollipop 123.

Step 1, FIG. 22B: The surgeon grasps the distal vessel 126 with a vascular clamp and inserts it into the distal side of the A/A 9 so that the trimmed end 127 touches the lollipop 123 and the distal balloon 126 extends fully into the lumen of the vessel 126.

Step 2, FIG. 22C: At this point, the surgeon double clicks his “ACTION” button 18, which results in the computer executing Steps 2, 3 and 4 shown in FIGS. 22C, D and E. Step 2 is the precise inflation of the distal balloon 125, which partially everts the end of the vessel 126 and presses it against the tines 105 and the curved portions 107 and 116 of the PAG 100 and Holder 103 assembly.

Step 3, FIG. 22D: The PAG 100 and PAG holder 103 assembly is translated axially in the proximal direction to “set” the tines 105 in the vessel wall 128. The distance translated is small: approximately one vessel wall thickness.

Step 4, FIG. 22E: The syringe 147 withdraws all the saline solution so that the distal balloon 125 collapses and withers. The lollipop 123 then translates in the proximal direction until the proximal balloon 125 is in position for receiving the proximal vessel 126. The syringe 147 then fills the proximal balloon 125 to the quasi-rigid state. The computer then reports “PROXIMAL READY” both verbally and by display on the control panels 7.

Step 5: Steps 1, 2, 3, and 4 are repeated with the proximal vessel 126. The lollipop 123 is then translated to a position midway between its distal and proximal positions with the balloons 125 deflated.

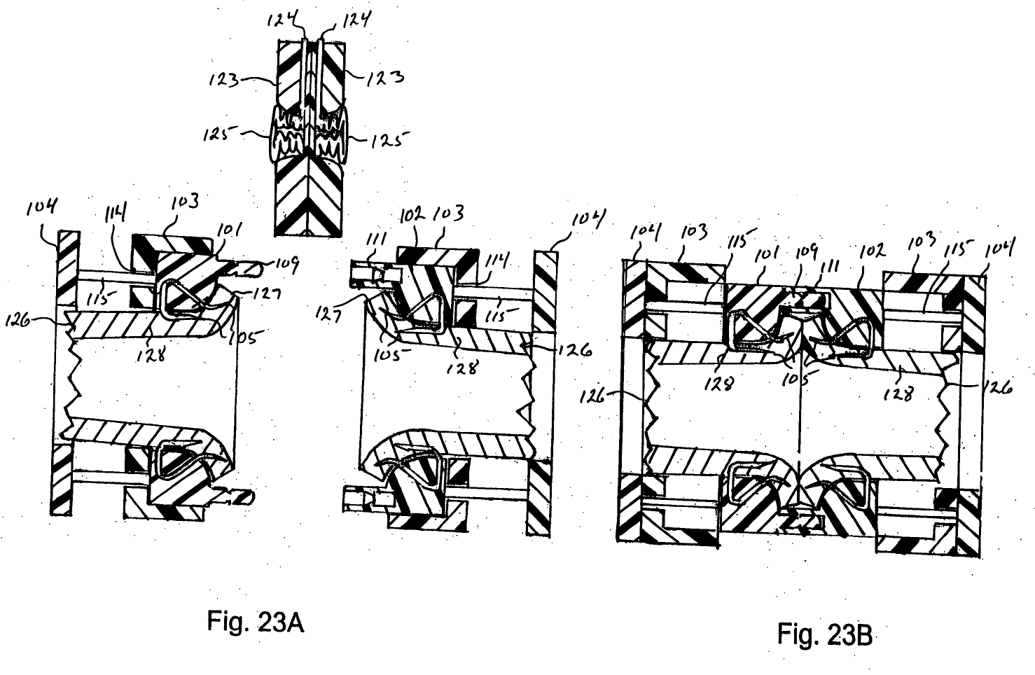

Step 6, FIG. 23A: The lollipop 123 is withdrawn as shown. When the lollipop reaches its stowed position, the computer then commands the proximal and distal PAG ejectors 104 to translate toward one another. Recall that the PAG ejectors 104 also carry the PAGs 100 and PAG holders 103 so that the entire two assemblies 156 translate toward the centerline of the A/A 9. The translation continues until the proximal 101 and distal 102 PAG connectors 108 are fully engaged and have snapped together. The two vessel ends 127 now have intima-to-intima contact and are held together by the PAGs 100 as shown in FIG. 23B.

Step 7, FIG. 23B: The computer now commands that the PAG holders 103 translate in opposite directions until the two holders 103 butt against their respective PAG ejectors 104. In the process, the connected PAGs 100 are ejected from their respective notches 113 in the holders 103. The connected PAGs 100 are now free of their holders 103 and the anastomosis is complete except for the removal of the PAG holder/ejector assemblies 156.

Step 8, FIGS. 24A and B: As previously mentioned, the PAG holders 103 and the PAG ejectors 104 have hinges 117 on either side that will allow the holders 103 and ejectors 104 to open unless they are otherwise restrained. As shown in FIG. 24A, this restraint is provided by horizontal restraint bars 129 that project from the inner wall of the A/A frame 130. During all the steps discussed thus far, the restraint bars 129 keep the holders 103 and ejectors 104 tightly closed as they translate back and forth through the first seven steps of the process. The A/A frame 130 also includes horizontal grooves 131 that engage the tongue projections 121 on each side of the ejectors 104. At this point, the computer commands that the proximal and distal PAG holder/ejector assemblies 156 be translated in opposite directions until they approach the proximal and distal ends of the A/A frame 130. At this location, there are no restraint bars 129 and the holder/ejector assemblies 156 open as shown in FIG. 24B.

Step 9: The surgeon grasps the surgeon's handle 25 and depresses the “ACTION” button 18 so that he can remove the used A/A 9 and move the wand 8 to a convenient location of his choice. The used A/A 9 is removed from the stylus, placed back in its container 300 and returned to its original position in the utility tray 10. The computer will sense through the bar card reader that the subject position in the utility tray 10 is occupied by a used A/A kit 16.

The completed anastomosis is depicted in FIGS. 25A and B. The blood vessel ends 127 are partially everted, held in apposition with intima-to-intima contact, and the intima have not been damaged in the process. An alternative embodiment of the SAS Type I A/A has the tines projecting from an annual collar which constitutes the appliance portion of the A/A. These collars are shaped precisely like the mod f PAG holder with the PAGs in place, and although this alternative embodiment is simpler and easier to fabricate, the segmented PAG 100/holder 103/ejector 104 approach just described has compelling advantages that justify its use. Some of these advantages are:

The blood vessels are free to pulsate at the graft line with little or no change in the vessel's pulsatile properties.

The vessel edges at the graft line are exposed to neighboring tissue and fluids to facilitate healing.

The segmented PAGs permit the blood vessel to grow with little or no reduction of patency. This is, of course, very important for the blood vessels of growing children.

The completed anastomosis is relatively easy to visually inspect.

The segmented PAGs permit the graft to be manually repaired and/or reinforced with conventional sutures if the surgeon desires.

Segmented PAGs have other advantages that are less obvious. For example, anastomoses that have graft lines at angles other than ninety degrees are easier to accommodate than with the collar approach. In addition, when the anastomosis involves blood vessels of different diameters, the segmented PAGs provide more flexibility in the resulting joint thereby mitigating the introduction of undesirable vessel angularity.

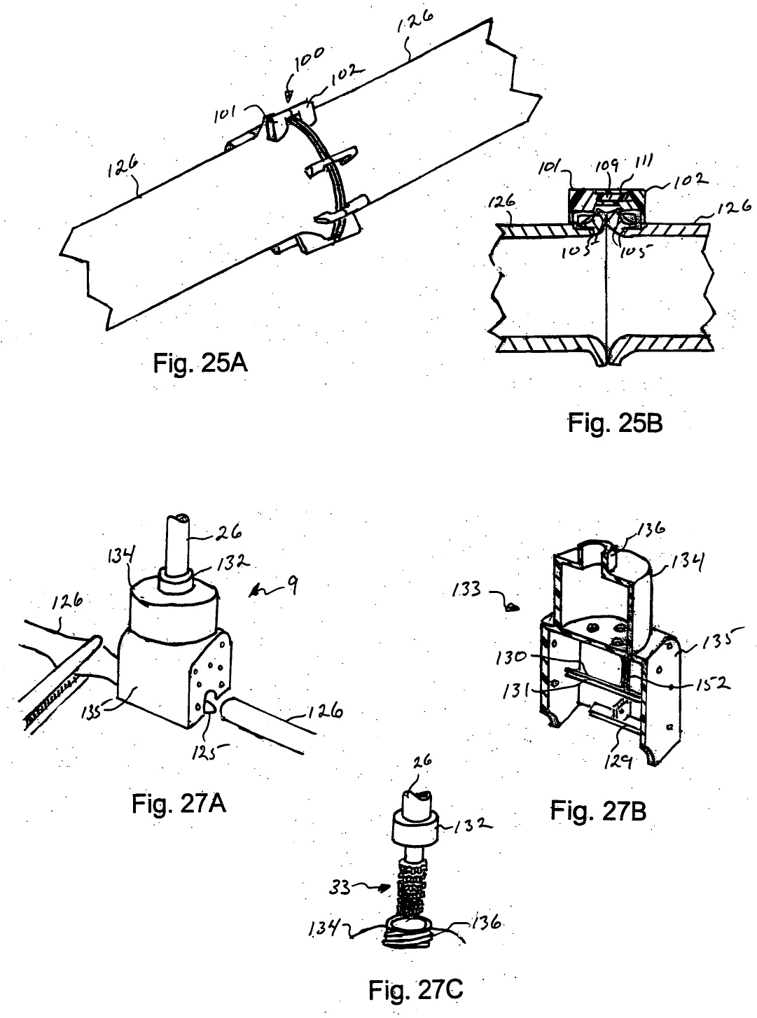

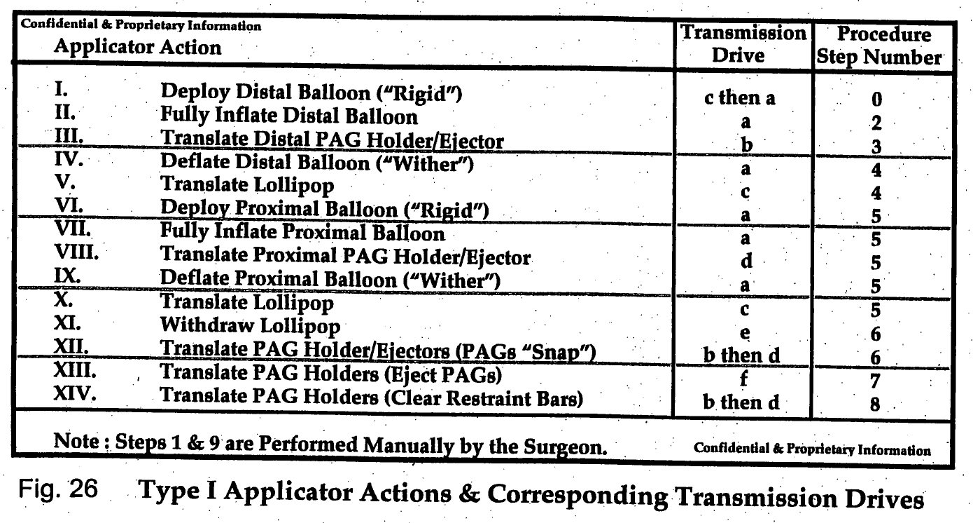

The applicator actions for the automated Type I anastomosis are listed in the chart of FIG. 26 along with the procedure step numbers shown in the previous figures. However, more important is the fact that these applicator actions are correlated to the transmission drive housed in the main body of the surgeon's wand. Therefore, these actions are performed by properly positioning the snubber and drive pinion relative to the transmission gears. The computer commands the control electronics that, in turn, control the timing and the process sequence.

The foregoing description has concentrated on the actual performance of a Type I anastomosis and the operations of the PAGs 100, PAG holders 103 and ejectors 104. These elements are part of the Appliance/Applicator 9 which is selected by the surgeon and attached to the end of the wand 8. All of the operations within the A/A 9 are driven by the concentric drive shafts 31 through the drive pinions 34 which connect to the A/A 9 to drive the actions of the lollipop 123 and the PAG holder/ejector assemblies 156 and their associated components.

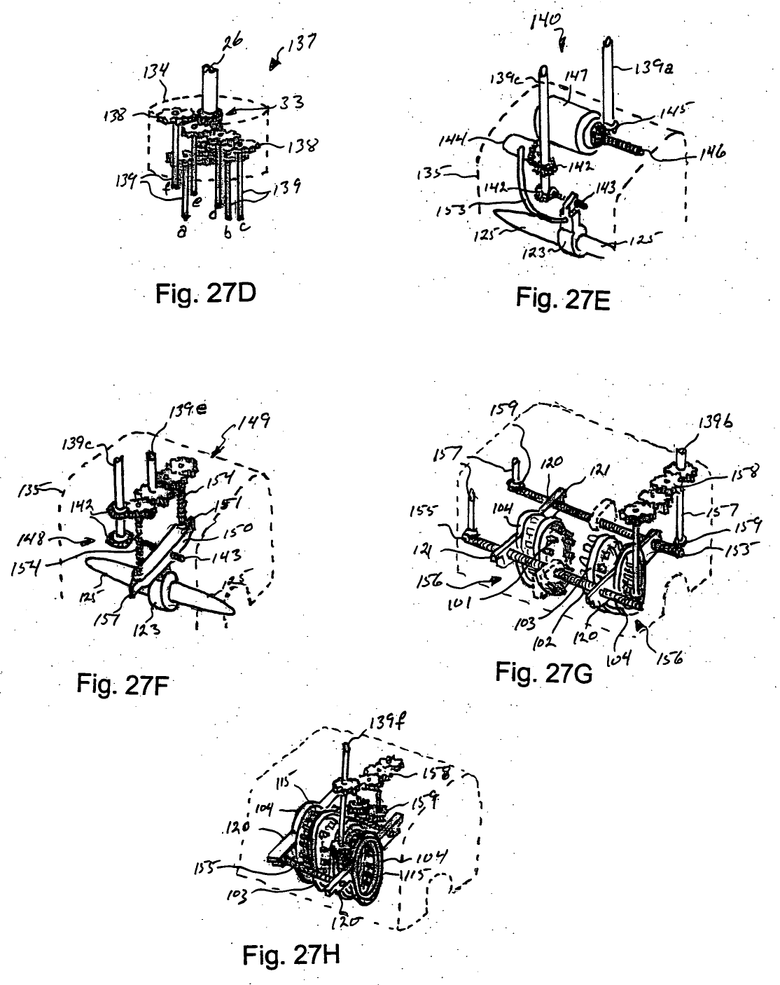

An example SAS Type I A/A embodiment will now be described. In the description, the relationship between the embodiment characteristics and the anastomosis actions will be discussed as well. This SAS Type I A/A embodiment is a micromechanical system whose various components are shown in FIGS. 27A-H.

FIG. 27A shows the A/A 9 attached to the end of the stylus 26 by means of a screw cap 132 with the ends of the vessel 126 to be anastomosed ready for insertion.

FIG. 27B is a cross section of the A/A housing 133 which is preferably injection molded of a suitable plastic material. This housing 133 is divided into two sub-housings: the transmission housing 134 and the appliance and manipulation housing 135. The housing 133 is also a primary structural element that supports the journal bearings, thrust bearings, pillow blocks, guide slots, restraint bars and other support and positioning elements associated with the various Applicator mechanisms.

FIG. 27C illustrates the wand connection in which the top end of the transmission housing 134 provides an interface with the stylus 26 on the surgeon's wand 8. It has a circular externally threaded hole 136, the inside diameter of the hole being such as to allow the six applicator drive gears 33 (pinions) at the tip of the stylus 26 to pass through, and the externally threaded diameter matches the diameter of the screw cap 132 on the stylus 26. As an alternative to the screw cap 132, a simple bayonet type connector, or similar structure, could be used so long as the result is that the A/A 9 is removably secured to the end of the Stylus 26 in a manner which provides for mechanical engagement of the applicator drive gears 33 with their corresponding elements within the A/A 9. The applicator drive gears 33 are driven by the actuators and transmission gears previously described inside the surgeon's wand 8. The screw cap 132 locks the stylus 26 and the A/A 9 together so that the applicator drive gears 33 are properly positioned within the transmission housing 134.

FIG. 27D illustrates the A/A drive transmission 137 located inside the transmission housing 134. The A/A drive transmission 137 mechanism interfaces with the six applicator drive gears 33 projecting from the tip of the stylus 26. This mechanism 137 consists of a set of six spur gears 138 and their output drive shafts 139. The spur gears 138 and drive shafts 139 are constructed of appropriate materials and designed to withstand the reaction forces exerted during the automated anastomosis procedure. Each spur gear 138 engages a specific applicator drive gear 33 when the screw cap 132 on the stylus 26 is tightened. The rotation of an applicator drive gear 33 (from the actuators in the surgeon's wand) causes rotation of the corresponding spur gear 138 and output drive shaft 137 thus transmitting rotational motion at the end of the drive shaft. The drive shafts are identified as a-f in FIG. 27C so they can be identified in the remaining Figures and correlated to the chart in FIG. 26.

FIG. 27E illustrates the everter syringe drive 140 and selector valve 141. The first two sequenced steps in FIG. 26 (Step 0 and Step 2) involve positioning the lollipop 123 and then deploying and inflating the balloon 125 for the distal blood vessel 126. These steps are performed by actuating transmission gear 37c in the surgeon's wand 8 by positioning the snubber vane 43 such that it locks all but gear 37c. The rotational actuator 36 then rotates through the predefined angle (usually a number of revolutions either clockwise or counterclockwise). This rotation is transmitted by one of the concentric shafts 31 to the applicator drive gear 33c at the end of the stylus 26 that, in turn, actuates the corresponding spur gear 138c and output drive shaft 139c in the A/A drive transmission 137. The output drive shaft 139c has bevel gears 142 attached at the distal end that (1) drive a lead screw 143 that translates the lollipop 123 to its “DISTAL” position and (2) actuate the selector valve 144 so that the saline solution will be directed to the distal balloon 125. Next, transmission gear 37a is automatically selected in the surgeon's wand 8 and the rotational actuator 36 rotates through a prescribed angle to rotate the appropriate output drive shaft 139a in the A/A 9. The distal end of this shaft 139a has a bevel gear/drive-nut 145 arrangement that drives a lead screw 146 to actuate the piston of the syringe 147. The prescribed rotation of the shaft deploys the distal balloon 125 to a “rigid” state. This action completes Step 0 of the process and the A/A 9 is ready for the anastomosis to begin. After the surgeon manually inserts the distal vessel 126 and double clicks his “ACTION” button 18, the automated process resumes. The rotational actuator 36 (that is still coupled to transmission gear 37a) rotates through a prescribed angle so that the distal balloon 125 is fully inflated. This action completes Step 2.

When Step 4 is reached later in the process, transmission gear 37a will be rotated again through a prescribed angle so that the distal balloon 125 is deflated (“withered”) and all saline solution has been returned to the syringe 147. Still later in the process, transmission gear 37a will be rotated again to deploy the proximal balloon 125 to its “rigid” state. Subsequently, the transmission gear 37a actions described above for the distal balloon 125 will be repeated with the proximal balloon 125.

It should not be necessary in this discussion to relate the remaining steps in the procedure to the actions of the actuators and the transmission gears 37 in the surgeon's wand 8. The remaining discussion will focus solely on the A/A 9 mechanisms since it should be very easy to relate the actions of the A/A 9 mechanisms to the computer-controlled actions of the actuators in the surgeon's wand 8.

FIG. 27F shows the everter translator 148 and retractor 149. The everter translator 148 translates the lollipop 123 in an axial horizontal motion. There are three functional positions that the lollipop 123 must assume during the anastomosis procedure: middle, distal and proximal. The axial motions are small but necessary in order for the lollipop 123 to be able the clear the PAG 100 connector mechanisms when it is retracted in Step 6.

Initially, the lollipop 123 is in the middle position and the selector valve 144 is closed so that saline solution cannot flow either into or out of the syringe 147. During Step 0, the drive shaft 139c translates the lollipop 123 to the distal position and puts the selector valve 144 in the distal position. At the end of Step 4, the lollipop 123 is translated to its proximal position and the selector valve 144 is turned to its proximal position. At the end of Step 5, the lollipop 123 and the selector valve 144 are returned to their respective middle positions. All translations of the lollipop 123 are relative to the fixed axial position of the retractor yoke 150 which cannot move axially since it has two tongues 151 that ride in the vertical guide slots 152 in the frame 130 (see FIG. 27B).

The flexible tubing 153 arrangement shown in FIG. 27E is for conceptual illustration only. An alternative design may utilize a sliding valve mounted to the frame 130 just below the retractor yoke 150. The sliding side of the valve would be rigidly connected to the strut that supports the lollipop 123 with the fixed portion of the sliding valve being connected to the syringe 147 with rigid hypodermic tubing. The sliding portion of the valve would have two ports connected through the lollipop strut: one port connected to the distal balloon 125 and one port connected to the proximal balloon 125. The sliding valve would be arranged so that the syringe 147 is connected to the distal balloon 125 when the lollipop 123 is in the distal position and connected to the proximal balloon 125 when the lollipop 123 is in the proximal position. In all other positions of the lollipop 123 (including the middle position), no saline solution can flow in either direction.

When the process reaches Step 6, the lollipop 123 is in the middle position and must be moved out of the way of the PAG/holder/ejector assemblies 156 so that the distal and proximal PAGs 101 and 102 can be connected. This is accomplished by rotating drive shaft 139e that causes two vertical lead screws 154 to raise the retractor yoke 150 (and the lollipop 123) a specified distance as commanded by the computer. Recall that at all times subsequent to Step 5, both balloons 125 are completely deflated (or “withered”).

FIG. 27G illustrates the PAG holder/ejector assembly 156 translation drive. In Step 3 and during Step 5, the distal and proximal PAG holder/ejector assemblies 156 are translated approximately one vessel wall thickness in order to “set” the tines 105 in the vessels 126. These assemblies 156 will be translated again in Step 6 to “snap” the distal and proximal PAGs 101 and 102 together and still again, in Step 8 to open the assemblies 156 so they can be removed. All of these translations are performed by means of the mechanisms depicted in FIG. 27G.

As previously described, the PAGs 100 are carried by their PAG holders 103 which are, in turn, carried by the PAG ejectors 104. Each PAG ejector 104 has a pair of projecting tabs 120 with tongues 121 that ride in the horizontal guide slots 131 in the frame 130. Each tab 120 has a threaded hole 122 that accommodates a lead screw 155 that is roughly half as long as the axial length of the frame 130 as shown in the Figure. In the example of FIG. 27G, two of the four lead screws 155 have right hand threads and the other two have left hand threads; other designs could easily have all the lead screws 155 identical.

Translation of the distal PAG holder/ejector assembly 156 is performed by rotating the drive shaft 139b which, in turn, rotates the two distal lead screws 155 through an arrangement of shafts 157, spur gears 158 and bevel gears 159 as shown. The proximal PAG holder/ejector assembly 156 translation drive is a mirror image of the distal drive mechanism and is driven by drive shaft 139d.

Since the drive mechanism chosen for the surgeon's wand 8 has only one output rotation, all steps in the procedure must be done sequentially. In Step 6, for example, the drive shaft 139b is rotated first to move the distal PAG holder/ejector assembly 156 to a position where its PAG connectors 108 are at the axial center of the A/A 9. Then the shaft 139d is rotated to actuate a similar arrangement of shafts 157, spur gears 158 and bevel gears 159 to move the proximal PAG holder/ejector assembly 156 in a distal direction until its PAG connectors 108 engage the distal PAG connectors 108, and the two sets of PAGs 100 snap together.

This translation mechanism also performs the final automated step in the process (Step 8). After the connected PAGs 100 have been ejected from their holders 104 in Step 7, the shaft 139b is rotated to move the distal holder/ejector 156 away from the center of the A/A 9 until it reaches the point where it no longer engages the restraint bars 129 on the frame 130. The shaft 139d is next rotated to move the proximal holder/ejector 156 until it no longer engages its restraint bars 129. Now all holder 103 and ejector 104 rings are open as shown in FIG. 24B and the surgeon can remove the A/A 9.

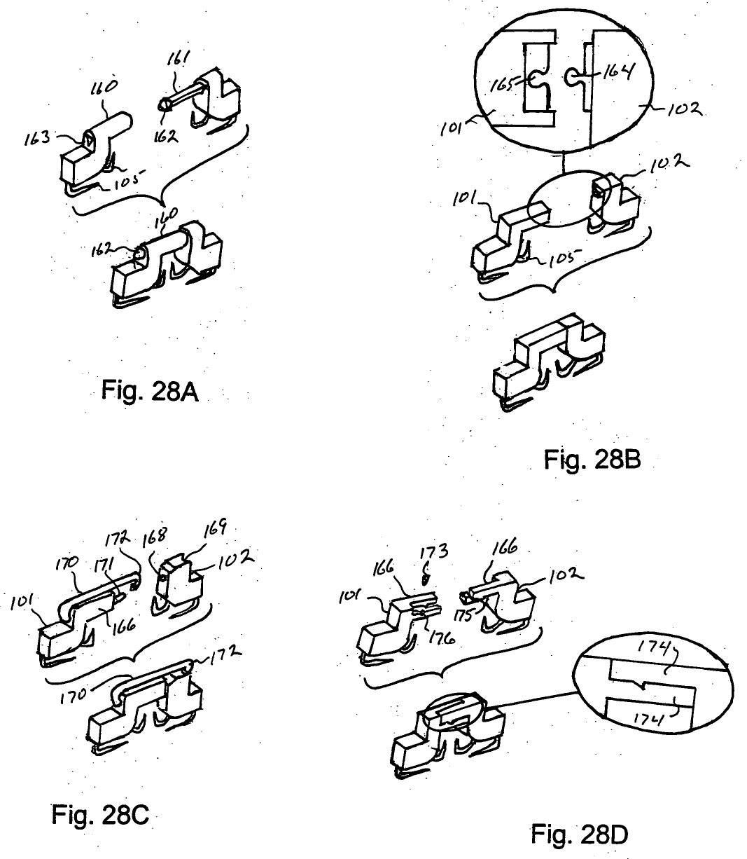

FIG. 27H illustrates the PAG ejector drive. As shown in FIGS. 23A and B, each PAG holder 103 is translated toward its ejector 104 so that the ejector pins 115 will push the PAGs 100 out of the two holders 103. This translation is accomplished by rotating drive shaft 139f, which, in turn, rotates the spur gear 158/bevel gear 159/lead screw 155 arrangements depicted in FIG. 28H. The four lead screws 155 translate the two PAG holders 103 toward their respective PAG ejectors 104, and the joined PAGs 100 are ejected. The following and final step of the automated process has already been described above in conjunction with FIG. 27G. Note that six drive shafts 139a-f were utilized in performing the sequential steps in the Type I anastomosis process. This is also the number of shafts required for Type II and Type III anastomoses; this fact will be demonstrated later in the description of the Type III A/A.

There are numerous approaches for snapping and locking the PAGs 100 during the automated anastomosis procedure. The PAGs 100 consist of two main components: (1) the main body 106 that holds the tines 105, sits in the PAG holder 103 and provides a guide for the correct eversion angle and (2) the PAG connector 108 extension that provides the locking mechanism. The previous description has been based on PAGs with a simple male-female connector 108 as has been previously described herein.

FIG. 28A-D illustrate PAGs 100 with alternative connector 108 structures and locking mechanisms presented as additional examples for use in the present invention. As shown, the main bodies 106 of the PAGs 100 are identical; however, the connector 108 extensions and locking mechanisms are quite different:

FIG. 28A shows a prismatic tube 160 and rod 161 snap connector. The triangular rod 161 goes through the triangular hole of the prismatic tube 160 and once it clears the hole, the snap head 162 engages a rear face 163 of the tube 160 to provide the locking mechanism.

FIG. 28B shows a bead 164 and groove 165 detent connector. The proximal PAG 101 has an extension component with a pad having a recessed plastic groove 165 as shown in the figure. The distal PAG 102 has a pad with a raised plastic bead 164 of the negative shape of the groove 165 but of smaller size. Locking is accomplished when the bead 164 is snapped into the groove 165.

FIG. 28C shows a stainless steel guide rod and clip connector. The face of the distal PAG 102 has a guide hole 168 and the back is molded with a clip groove 169 to accommodate the stainless steel clip 172. The extension part 166 of the proximal PAG 101 has a steel rod 170 molded in it. One end of the steel rod 170 extends from the PAG 101 to provide a guide 171 for mating with the hole 168 on the distal PAG 102. The other end of the steel rod 170 is bent over the extension part to create a spring clip 172 shape as shown in the figure. During the locking process the end of the spring clip 172 rides over the distal PAG 102 and when the distal 102 and proximal 101 PAGs come together, the end of the clip 172 springs into the distal PAG's clip groove 169 thus providing a lock holding the two PAGs together.

FIG. 28D shows a prismatic cruciform detent connector 173. The proximal and distal PAGs 101 and 102 for this type of connector are identical. The extension parts 166 are mirror images of each other and shaped as diagonally opposed elongations 174 as shown in the figure. There are detent tongues 175 and detent grooves 176 that engage when the elongations 174 interdigitate to provide the locking mechanism.

It is apparent that the eversion balloons 125 are essential features of the SAS appliance/applicators. It is equally apparent that these balloons 125 become very small when an A/A 9 is designed for blood vessels 126 with small inside diameters. Latex and silicone rubber balloons 125 have been successfully fabricated with various shapes and with outside diameters as small as one millimeter. The fabrication technique involved either brass or steel mandrels turned to the desired shape of the inside of the balloon. The mandrel was coated with a separation compound and then painted with a thin coat of uncured latex or silicone RTV. After partial curing, successive coats were selectively applied until the desired distribution of balloon thickness was achieved. After complete curing, the balloon was lightly dusted with talc, stripped from the mandrel, (and turned inside out in the process). After another dusting of talc, each balloon was stored in a cool, dry environment until it was needed for test purposes.

A simple apparatus is used for testing balloons and includes an accurate micrometer drive that actuates the piston of a conventional medical syringe. The syringe used for testing displaces 0.04 milliliters of volume per millimeter of piston translation, but syringes of different sizes can be used if desired. The syringe is connected to a test fixture that clamps the balloon in place and provides a leak-free passageway for the input/output of the syringe. A number of test fixtures with various sizes and shapes were used in the tests.

In practice, the micrometer is set so that the piston of the syringe is approximately at its midpoint. The balloon is then clamped in place on the detached test fixture. The balloon, the passageway and the syringe are then filled with saline solution with a hand-operated syringe. The test fixture is carefully re-attached to the syringe and sealed; there must be no air left in the test system.

A typical test sequence involves translating the syringe piston to a number of positions with the micrometer drive. At each position, a vertical and a horizontal photograph is taken of the balloon with a grid in the background so that the size and shape of the balloon can be determined for each micrometer setting. One of the micrometer settings must be for zero volume of solution in the balloon (the balloon is “withered”). All micrometer positions relative to this zero position can then be converted into the volume of solution in the balloon for each photograph.

As might be expected, the appliance/applicator for performing Type III anastomoses is considerably more complex than the Type I A/A. However the principles and methods used by each type are the same. Consequently, the discussion and comments concerning the Type III A/A can be brief since the figures tend to be self-explanatory to someone who understands the previous Type I illustrations and discussions.

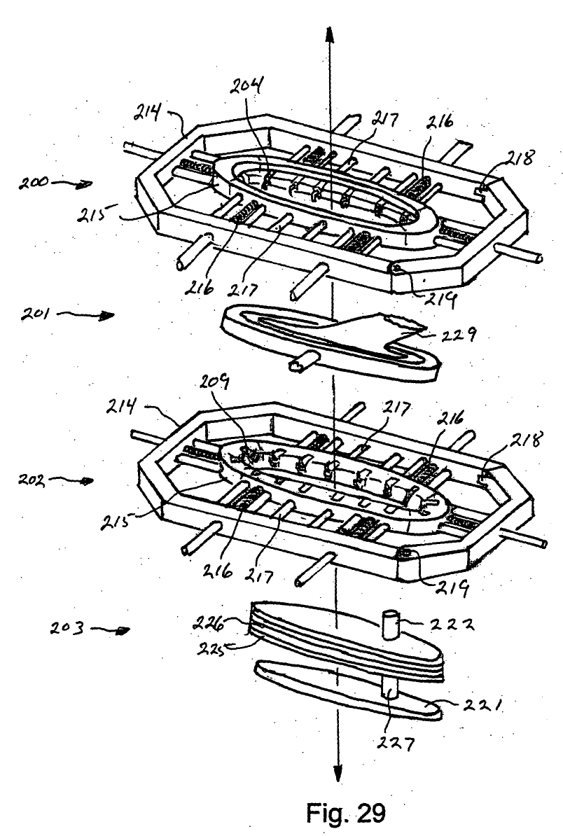

The Type III anastomosis was defined as end-to-side in FIG. 1C. The Type III A/A 9 consists of the following major assemblies: The end graft PAG holder/ejector 200, the end graft everter 201, the side graft PAG holder/ejector 202 and the side graft punch/everter shoe 203. The name given to each assembly relates to the main function carried out. The component parts that make up each of these assemblies correspond to the same parts as in the Type I A/A previously described and are shown in FIG. 29. The Type III example chosen for discussion involves a thirty degree angle between the side graft and end graft vessels which accounts for the unusual shape of the end graft everter balloon 229 depicted here although other angular measurements are within the scope of the present invention.

As in a Type I anastomosis, the Type III PAGs 204 and 209 and holder/ejector assemblies 200 and 202 are mechanisms that hold and manipulate the PAGs 204 and 209 in order to perform the Type III procedure. There are two of these assemblies: one for the end graft vessel 231 and one for the side graft vessel 230. The two assemblies are substantially the same in structure, functionality and operation and only one will be described. Any differences between the two assemblies will be noted during the subsequent discussion.

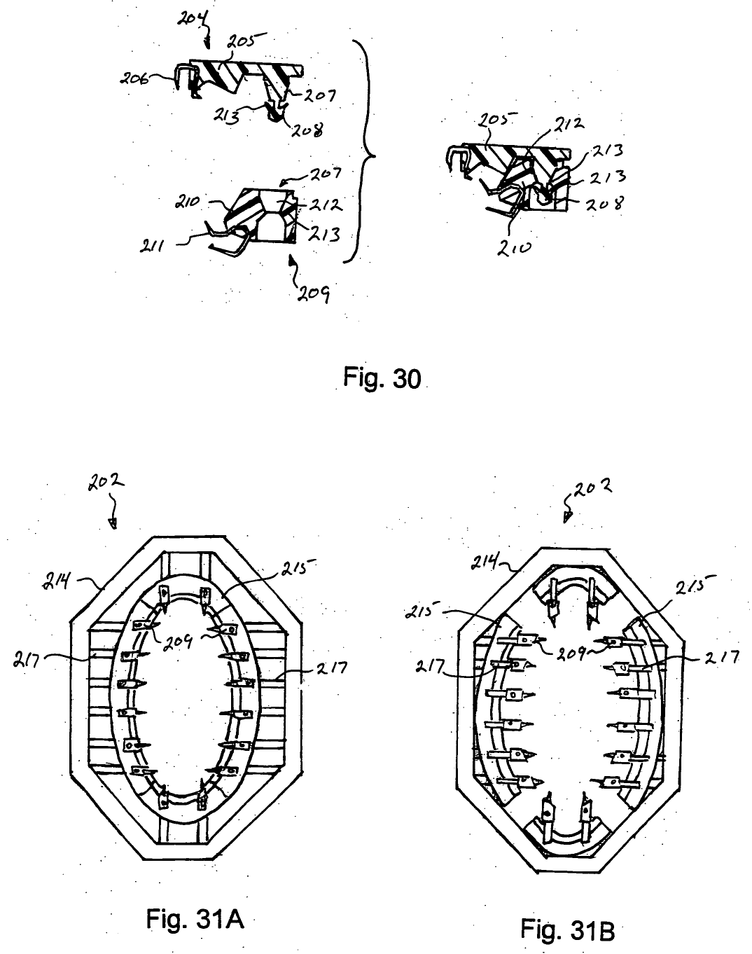

The principal difference between the end graft assembly 200 and the side graft assembly 202 are the respective PAGs 204 and 209 shown in cross section in FIG. 30. Because of the angular relationship between the end graft vessel 230 and the side graft vessel 231, the PAGs 204 and 209 have different relative structures so as to correctly place the tines 206 and 211 into the respective blood vessel walls. However, they also lock together to provide securement of the anastomosis. The end graft PAGs 204 consist of the PAG main body 205 with two tines 206 projecting from it plus a connecting mechanism 207. The tines 206 are sharpened to provide for easy penetration of the blood vessel 231 outer layer. The tines 206 are shaped to provide for penetrating the outer layer of the blood vessel 231 at two locations. The PAG main body 205 holds the tines 206 and is shaped to provide an eversion angle and a radius that do not damage the blood vessel 231 during the eversion process. The other end of the main body 205 of the PAG 204 is shaped to provide the male part 208 of the connecting mechanism 207.

The side graft PAGs 209 also consist of the PAG main body 210 with two tines 211 projecting from it plus a connecting mechanism 207. The tines 211 are shaped to provide for penetrating the outer layer of the side graft blood vessel 230 at two locations. The PAG main body 210 holds the tines 211 and is shaped to provide an eversion angle and a radius that do not damage the blood vessel 230 during the eversion process. The other end of the main body 210 of the PAG 209 is shaped to provide the female part 212 of the connecting mechanism 207.

As with the Type I PAGs 100 the main bodies 205 and 210 of the end and side graft PAGs 204 and 209 are shaped such that they mate with each other and remain in this state once locked together. The connecting mechanism 207 depicted here is one example of the multiplicity of connector designs that could be used to join PAGs 204 and 209 in the manner suggested with the Type I PAGs 100. In this example, the male part 208 is adapted to fit into the female part 212 and includes a detent mechanism 213 which locks the end and side graft PAGs 204 and 209 together. This detent mechanism 213 may be any such mechanism that is moldable into plastic by injection molding and that serves to securely hold the PAGs 204 and 209 together following completion of the anastomosis.

As shown in FIG. 29, each of the graft PAG holder/ejector assemblies 200 and 202 consists of an outer 214 and inner 215 ring. The outer ring 214 is the ejector ring that provides support for the driving lead screws 216 and ejector pins 217. The outer ring 214 is made of two components that are held together through a hinge 218 and locking mechanism 219. Once the locking mechanism 219 is released, one of the parts is allowed to rotate about the hinge 218 to allow for the removal of the assemblies 200 and 202 once the anastomosis is completed.

The inner ring 215 or PAG holder ring holds and guides the PAGs 204 and 209 for each graft (end and side) during the anastomosis procedure. The inner ring 215 is segmented at four places. The four parts comprising the inner ring 215 are held at their correct locations through the positioning of the driving lead screws 216 supported by the outer ring 214. The segmentation of the inner ring 215 is designed to not interfere with the outer ring 214 once the PAGs 204 and 209 are ejected and allows for its removal once anastomosis is completed.

The Type III anastomosis connects the end of a blood vessel 231 to the side of another blood vessel 230. This end-to-side type of connection guides the shape of the inner 215 and outer 214 rings. The inner ring 215 is shaped in an oval or elliptical shape to increase the perimeter and attachment area of the blood vessels 230 and 231. The outer ring 214 shape is roughly similar in order to provide the support structure for the lead screws 216 and ejector pins 217.

The Type III side graft PAG holder/ejector assemblies 202 have been generally described in connection with FIG. 29, so the focus now will be the PAG ejector process.

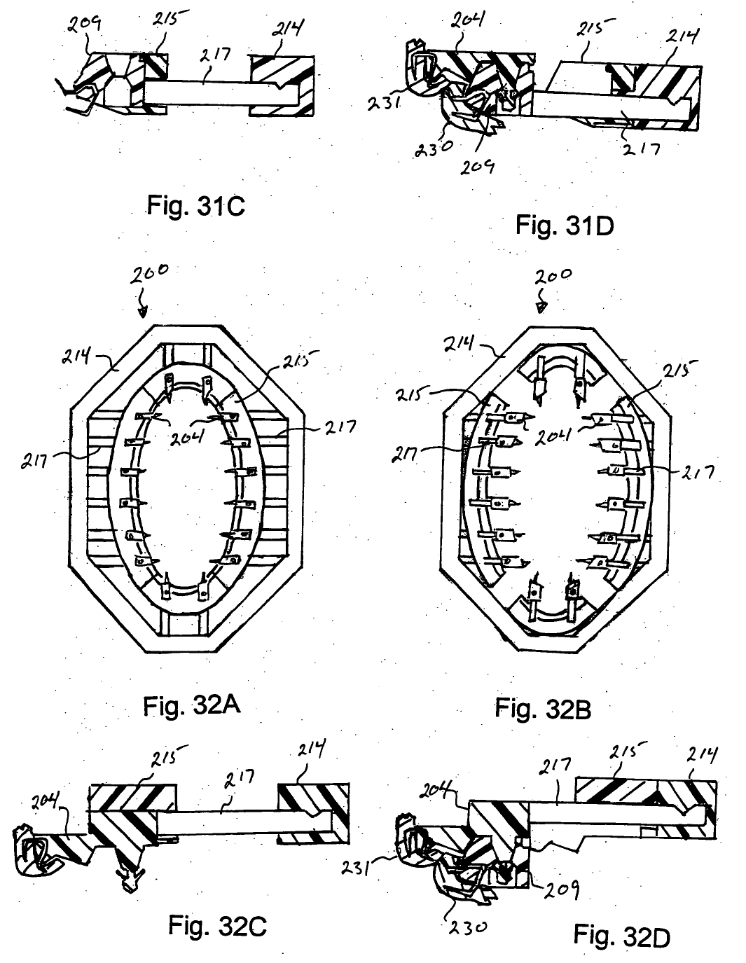

A top view of a side graft PAG holder/ejector 202 is shown in FIG. 31A. The PAG holder or inner ring 215 is made in four segments as shown, and this view shows the PAGs 209 pressed in place in the holder 215. The lead screws 216 (not shown) and the ejector pins 217 are holding the four segments in place. After several steps that will be discussed later, the side graft PAGs 209 will be snapped together with the end graft PAGs 204 as shown in the cross-sectional views 31C and D. The PAGs 209 are then ejected from the holder by lead screws 216 as shown in FIG. 31B.

The Type III mod f end PAG holder/ejector assemblies 200 operate in a manner similar to the side graft PAG holder/ejector assemblies 202 discussed above and as shown in FIGS. 32A-D Thus, a top view of an end graft PAG holder/ejector 200 is shown in FIG. 32A and structurally is substantially identical to the side graft PAG holder/ejector assembly 202. Accordingly, like elements are numbered the same. Like the side graft PAG holder/ejector assembly 202, the end graft PAG holder or inner ring 215 is made in four segments as shown, and this view shows the PAGs 204 pressed in place in the holder 215. The lead screws 216 (not shown) and the ejector pins 217 are holding the four segments in place. After several steps that will be discussed later, the end graft PAGs 204 are snapped together with the side graft PAGs 209 as shown in the cross-sectional views 32C and D. The PAGs 204 are then ejected from the holder by lead screws 216 as shown in FIG. 32B.

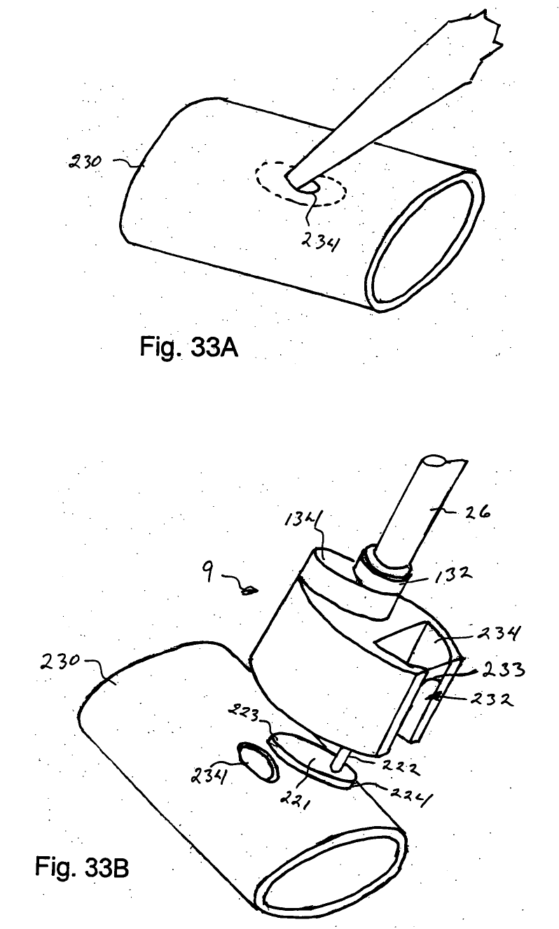

The A/A 9 to be used in the Type III procedure is defined by: (1) the inside diameter and wall thickness of the side graft vessel 230, (2) the inside diameter and wall thickness of the end graft vessel 231 and (3) the angle desired between the end graft 231 and side graft vessels 230. In this example, an angle of thirty degrees was chosen for illustration purposes. Other angles may be chosen depending on the vessels to be anastomosed and are within the scope of the present invention. The Type III procedure and the operation of the Type III PAG holder/ejector assemblies 200 and 202 will be described in conjunction with FIGS. 33-40.

FIG. 33A: Both the side graft 230 and end graft 231 vessels are exposed by traditional means. The side graft vessel 230 is then temporarily occluded proximally and distally by clamps, ligatures or other suitable means. In Step 1 of the anastomosis procedure the surgeon makes a small incision 234 through the side graft vessel 230 wall at the point where the anastomosis is to occur. The surgeon now enters into the computer the estimated diameter and wall thickness of the side 230 and end graft 231 vessels and the angle desired between them. The computer calculates and then indicates the location of the proper A/A kit 16 in the utility tray 10 that will accomplish the specified anastomosis. The A/A kit 16 is opened and the A/A 9 is installed at the tip of the stylus of the surgeon's wand 26 in the same manner as described in connection with the Type I device.

Step 2, FIG. 33B. The Type III A/A 9 contains all the mechanisms described in FIG. 29. The ejector ring 214 of the side graft PAG holder/ejector assembly 202 is flush with an opening in the bottom of the A/A appliance and manipulation housing 135. The punch anvil 221 on the tip of the punch/everter strut 222 is extended a short distance below the A/A housing 135 as shown in FIG. 33B. Holding down the ACTION button 18, the surgeon inserts the toe 223, and then the heel 224, of the punch anvil 221 into the incision 234 so that the punch anvil 221 is in the lumen of the side vessel 230. In Step 3, the side graft ejector ring 214 is lowered until it is pressing against the side graft vessel 230.

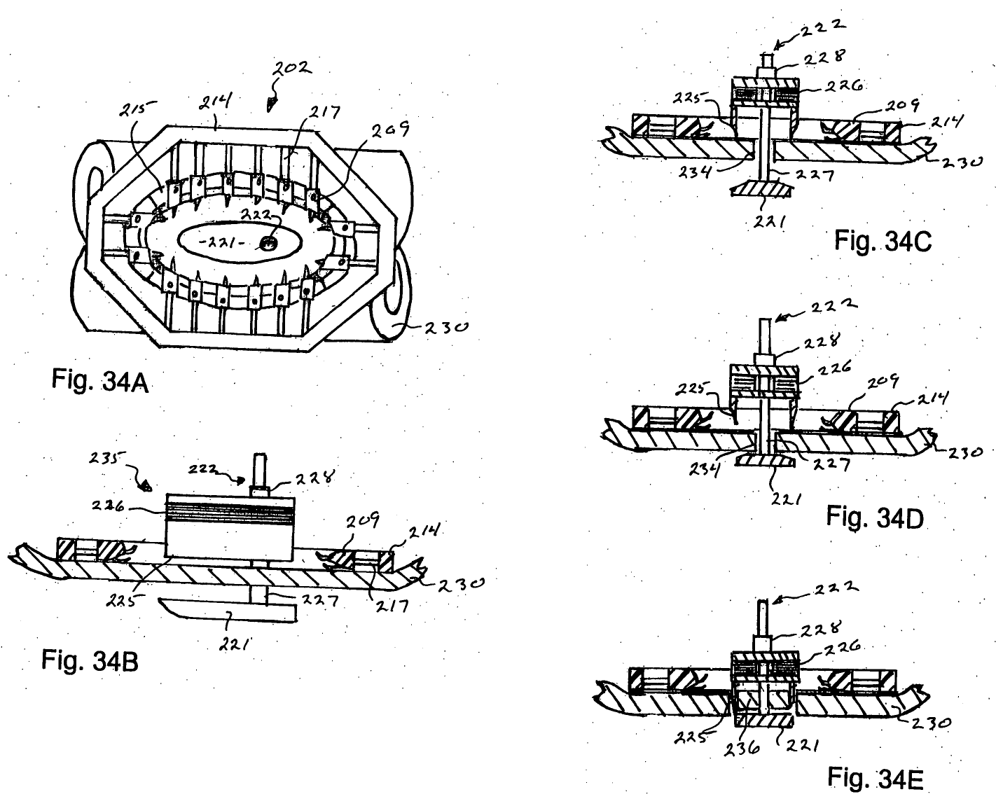

Step 4: At this point, the surgeon releases the ACTION button 18 and the A/A 9 is now rigidly held with the side graft ejector ring 214 pressed flush against the side graft vessel 230 at the desired location for the anastomosis. FIGS. 34A-E illustrate the procedure for preparing the side graft vessel 230 for eversion and show the components that constitute the side graft punch and everter shoe 203: the punch anvil 221, the punch blade 225, the stowed annular everter balloon 226 and the punch/everter strut 222. The punch/everter strut 222 is constructed of two telescoping stainless steel tubes. The inner tube 227 supports the anvil 221 and provides the means for raising and lowering the anvil 221. The outer tube 228 supports the punch bade/everter balloon assembly 235 and provides the means for raising and lowering that assembly. The inner tube 227 also acts as the conduit for the saline solution that will be used for inflating the balloon 226 in later steps. FIG. 34A is a view looking downward through the side graft holder/ejector 202 at this point in the procedure.

Step 5: With the A/A 9 in place and the ejector ring 214 pressed against the side graft vessel 230 as shown in FIG. 34B, the surgeon double clicks his ACTION button 18 and the automated sequence begins. In FIG. 34C the computer commands the Anvil 221 to move upward until it is approximately one blood vessel wall thickness below the lower edge of the punch blade 225 which, itself is flush with the bottom of the ejector ring 214. This position is shown in FIG. 34D.

Step 6, FIG. 34E: The computer then commands that the punch blade/everter balloon assembly 235 move down until the sharpened punch blade 225 rests firmly against the anvil 221. The punch blade 225 has the shape of a thirty-degree ellipse and its size is such that the resulting arterio/venotomy matches properly with the thirty-degree ellipse formed by the end of the trimmed end graft vessel 231. Note that the severed portion 236 of the vessel wall is trapped within the blade 225 by the anvil 221 and will remain there throughout the subsequent steps of the procedure.

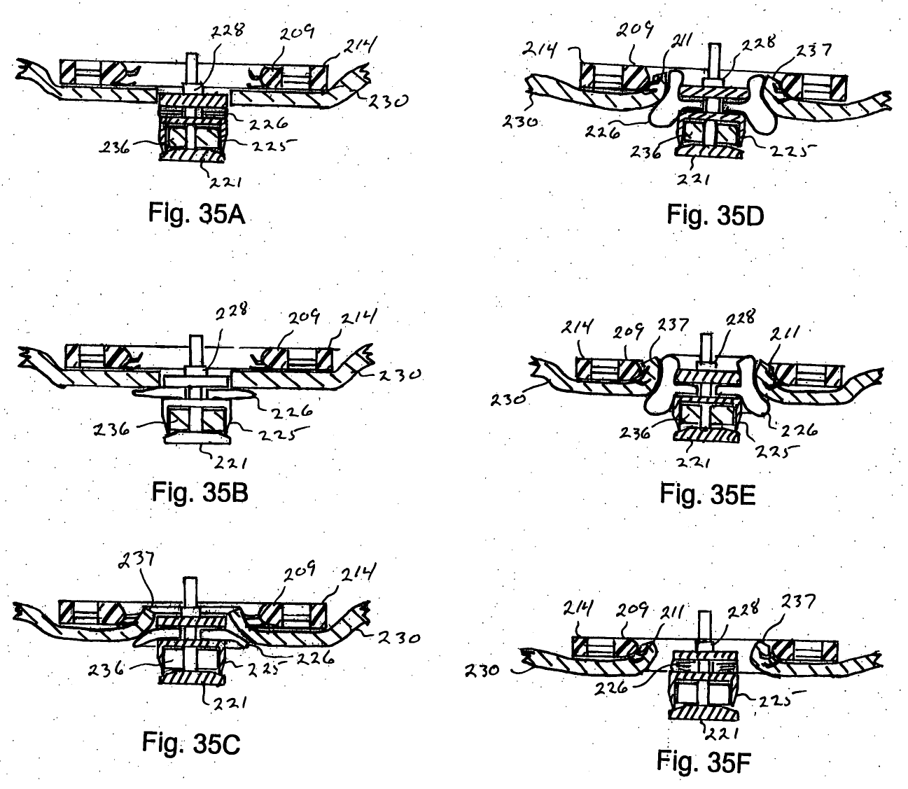

The next stage of the procedure is the eversion of the side graft vessel 230 wall and its engagement with the side graft PAGs 209. This stage is illustrated in FIGS. 35A-F.

Step 7: Following the arterio/venotomy described above, the Anvil 221 and punch/everter assembly 235 now move downward as a unit a distance that is approximately twice the vessel wall thickness so that the annular everter balloon 226 is just below the vessel wall 230 as shown in FIG. 35A.

Step 8, FIG. 35B: The computer now commands that a prescribed volume of saline solution be injected through the inner tube into the side graft annular everter balloon 226 so that it deploys and assumes its rigid state as shown. This rigid state is comparable to the rigid state of the everter balloons 125 in the Type I device previously described except that the annular everter balloon 226 has a shape resembling a flattened doughnut.

Step 9: Next, the entire anvil 221 and punch/everter assembly 235 moves upward a distance equal to one vessel wall thickness. This small movement slightly everts the edge of the arterio/venotomy as illustrated in the FIG. 35C and positions the balloon 226 for the next step.

Step 10: The computer now commands that a prescribed additional volume of saline solution be injected into the side graft annular everter balloon 226 so that the balloon 226 is in its fully inflated state as shown in FIG. 35D. This fully inflated state presses the edge 237 of the arterio/venotomy against the PAGs 209 & PAG holder ring 215 so that the edge 237 has the proper distribution of partial eversion around the periphery of the arterio/venotomy. At this point, the tines 211 have started penetrating the outer surface of the vessel 230.

Step 11: The PAG holder/ejector assembly 202 is now raised a distance equal to one vessel thickness as shown in FIG. 35E. The anvil 221 and punch/everter assembly 235 do not move during this step. This motion is to “set” the tines 211 in the blood vessel wall and to complete the eversion of the edge of the arterio-venotomy 237. Note that the shape and position of the tines 211 relative to the PAG main body 210 limits the penetration of the tines 211 so that there is no damage to the intima.

Step 12: The computer now commands that all saline solution be withdrawn from the side graft balloon 226 so that the balloon 226 resumes its “withered” state as shown in FIG. 35F. The vessel wall is held in its everted state by the tines 211 on the PAGs 209.

Step 13: The anvil 221 and punch/everter assembly 235 are raised until they are clear of the PAG holder/ejector assembly 202, and are then retracted to their stowed position inside the A/A housing 135 where they cannot interfere with subsequent actions. The computer next commands that a prescribed volume of saline solution be injected into the end graft everter balloon 229 so that it assumes its rigid state. The computer indicates audibly and by control panel display that the first automated sequence of the Type III procedure has been completed and that it is ready for the second and final automated sequence to begin.

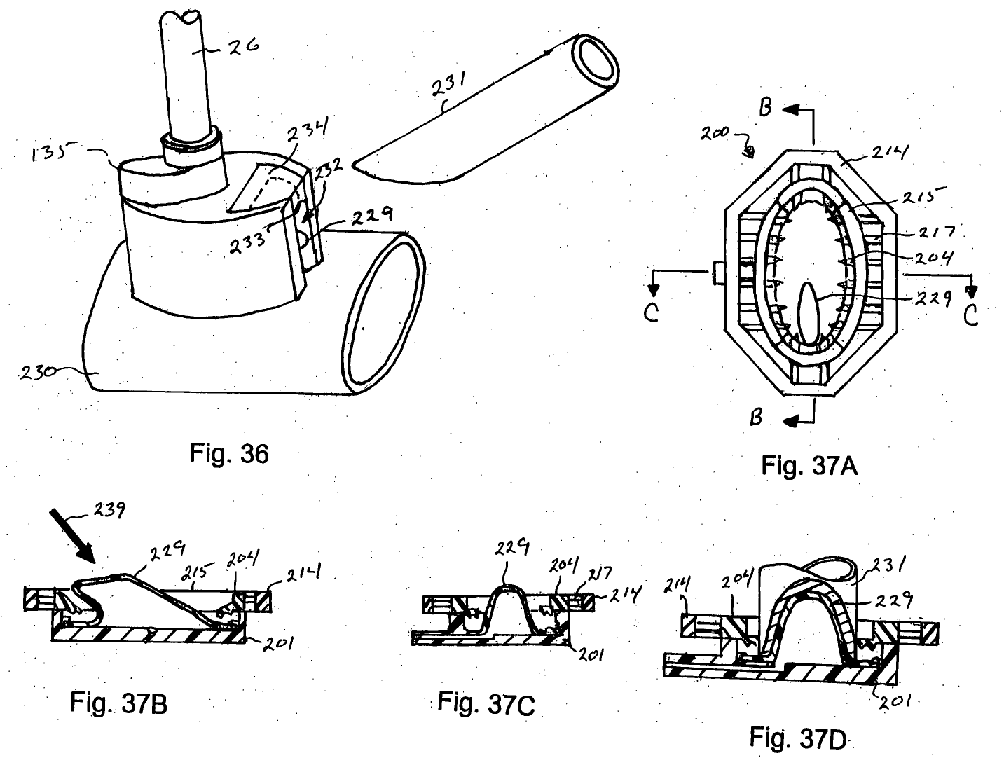

FIG. 36 illustrates this point in the procedure. Side graft arterio/venotomy has been completed and the Type III A/A 9 is frozen in position with the side graft PAG holder/ejector assembly attached by the PAGs 209 to the side graft vessel 230. The PAGs 209 have not yet been ejected from their holder and are retaining the eversion of the edge 237 of the side graft arterio/venotomy. The A/A 9 is now ready to receive the end graft vessel 231.

Step 14: The surgeon grasps the end graft vessel 231 with a vascular clamp and inserts the thirty-degree trimmed end 238 into the end graft opening 232 on the side of the A/A 9. The top of this opening has a short length of semi-circular wall 233 that guides the inserted vessel 231 at a thirty-degree angle relative to the side graft vessel 230. The short length of wall 233 is followed by the tip of the end graft everter balloon 229 so that the balloon 229 will enter the lumen of the inserted vessel 231. The base of the end graft everter 201 will limit the insertion distance to the proper amount. FIGS. 37A-D illustrate the end graft PAG holder/ejector assembly 200 within the A/A 9 in the present condition ready to receive the end graft vessel 231. The end graft everter balloon 229 is in its rigid state to facilitate insertion into the lumen of the end graft vessel 231 with arrow 239 in FIG. 37B showing the line of insertion of the end graft vessel 231. The housing 135 of the Type III A/A 9 includes a transparent window 234 above the end graft opening 232 so the surgeon can see that the end graft vessel 231 is properly inserted and seated over the end graft everter balloon 229. FIG. 37D shows the end graft everter 201 with the end graft balloon 229 in rigid form and the end graft vessel 231 in place ready for eversion.

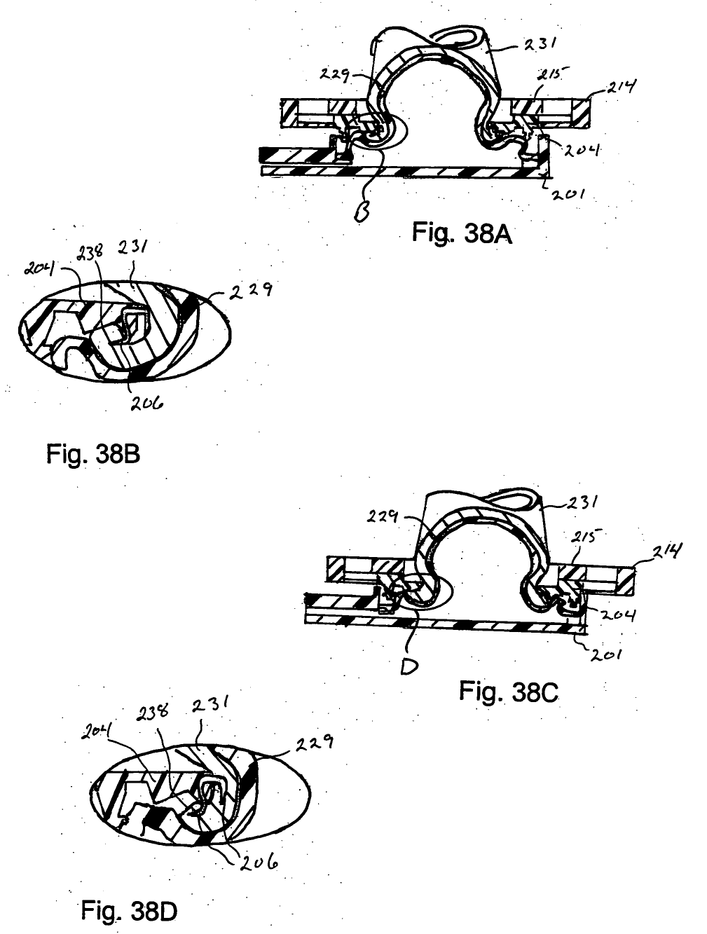

Step 15: With the end graft vessel 231 properly inserted into the A/A 9, the surgeon double clicks his ACTION button 16 to start the second automated sequence of actions, which is shown in FIG. 38A-D. The computer commands that a prescribed volume of saline solution be injected into the end graft everter balloon 229 so that the balloon 229 assumes its fully inflated state as shown in FIG. 38A. FIG. 38B is a close-up of the trimmed end 238 of the end graft vessel 231 showing the initial insertion of the tines 206 of the end graft PAG's 204 into the end graft vessel 231 wall as the result of the full inflation of the balloon 229.

Step 16: During the inflation process, the balloon 229 pushes the surrounding blood vessel 231 to conform to the shape of the end graft PAG 204 and holder or inner ring 215. At the computer's command, the end graft everter assembly 201 is raised by a distance approximately equal to the wall thickness of the end graft vessel 231 to the position shown in FIG. 38C. This motion sets the end graft tines 206 into the wall of the end graft vessel 231, as shown in the close up FIG. 38D, so that vessel wall will retain the proper everted shape after the balloon 229 has been deflated and removed.

Step 17: Following this, the computer now commands that the saline solution be removed from the end graft everter balloon 229 so that the balloon 229 assumes its withered state. Next, the end graft everter assembly 201 is lowered and then retracted into its stowed position in the A/A housing 135.

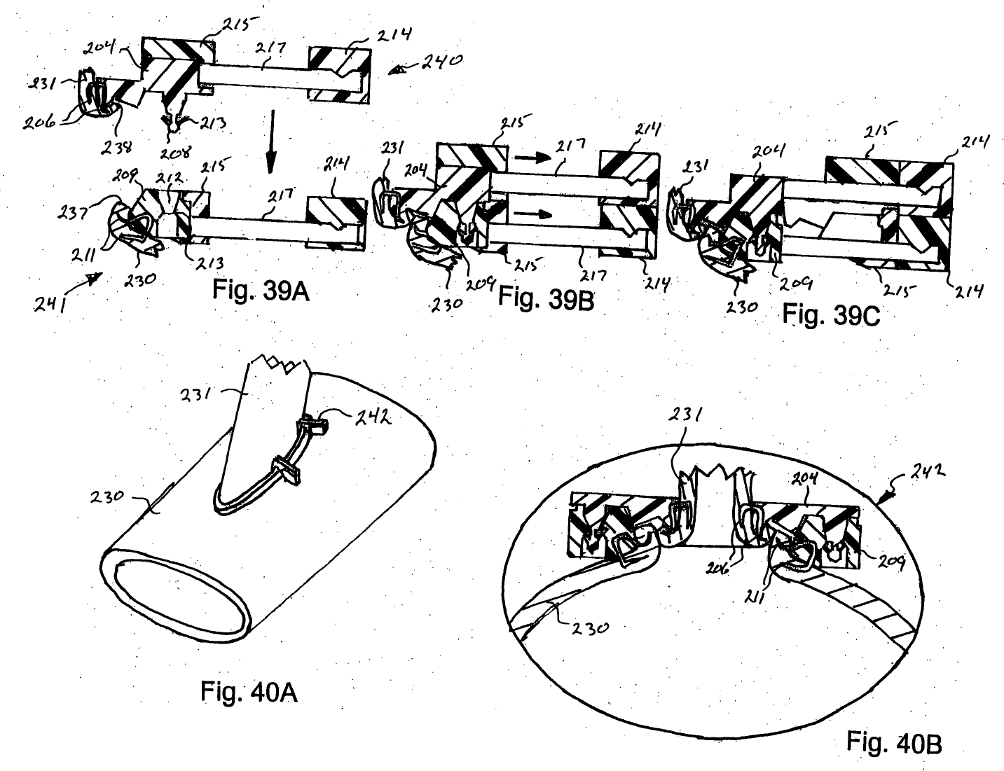

The Type III anastomosis is completed by the connection of the end graft and side graft PAGs 204 and 209 as shown in FIG. 39A-C.

Step 18: In FIG. 39A the end graft assembly 240 is lowered toward the side graft assembly 241 until the end graft PAGs 204 snap into the side graft PAGs 209 with the detents 213 of the respective male 208 and female 212 parts of the PAGs 204 and 209 engaging, thus locking the end and side graft PAGs 204 and 209 together as shown in FIG. 39B.

Step 19: Subsequently, the PAG holders 215 for the side 209 and end 204 graft PAGs are translated in unison, as shown in FIG. 39B by their respective lead screws 216 until they are flush against the PAG ejector rings 214. This action pushes the PAGs 204 and 209 against the ejector pins 217 that are rigidly held in place in the ejector rings 214 so that all PAGs 204 and 209 are ejected as the segments of the PAG holders 215 are translated. Now all the connected PAG pairs are free from the A/A 9 as shown in FIG. 39C.

Step 20: As already mentioned, one side of each side and end graft PAG ejector ring 214 is hinged on one side and held in place by a retaining pin or locking mechanism 219 (see FIG. 29). At the end of the translations of the PAG holder segments described above, the final motion of the segments nearest the locking mechanisms 219 trip pins so that both ejector rings 214 are free to swing open. The computer indicates both audibly and by control panel display that the anastomosis is complete. The surgeon depresses the ACTION button 18 on his wand 8 and withdraws the applicator 9. He then removes the applicator 9 from the stylus 26 and returns it to the A/A container 300 on the SAS utility tray 10.

A perspective and a cross-sectional view of a completed Type III Mod f anastomosis is shown in the FIGS. 40A and B respectively. Only two of the connected PAG pairs 242 are shown for illustrative purposes whereas an actual anastomosis would have eight to sixteen of these pairs.

The completed Type III Mod f anastomosis using segmented PAGs has certain advantages as compared to PAGs on an annular ring that remains in the body. These advantages are the same as those for a Type I anastomosis as previously discussed; however, these advantages are repeated here for emphasis:

Allows blood vessels to pulsate at the graft line with little change in the pulsatile properties.

Exposes the edge of the graft line to neighboring tissue and fluids to permit rapid healing.

Permits blood vessel growth with little or no reduction in patency.

Allows for better visual inspection of the completed anastomosis.

Allows manual reinforcement or repair with conventional sutures.

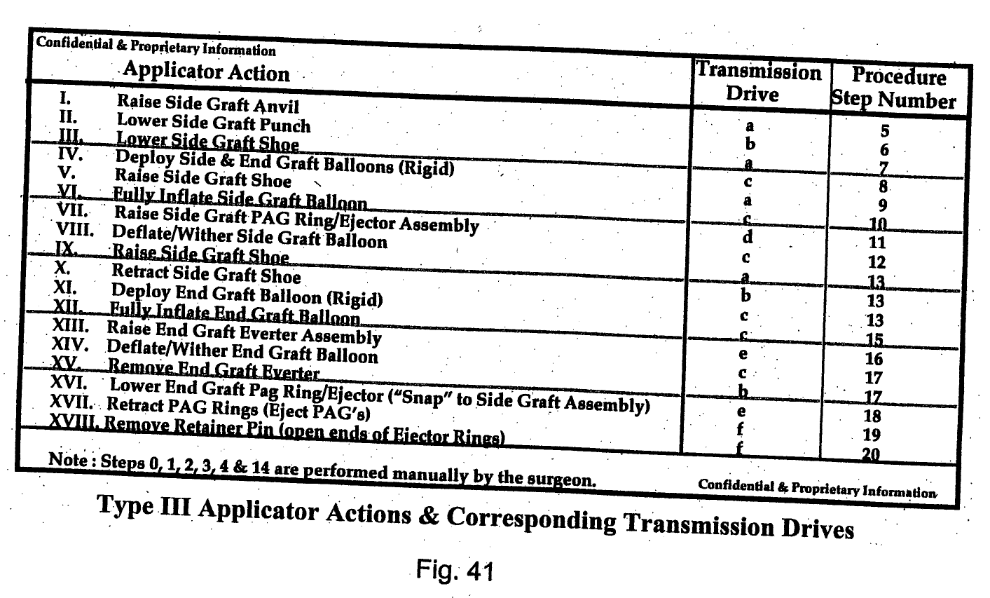

The execution of a Type III anastomosis involves twenty-one sequential steps as presented in the preceding figures. Of these twenty-one steps, fifteen are accomplished in an automated manner by the applicator portion of the A/A 9. The remaining six steps are performed by the surgeon. The applicator performs its fifteen steps by means of eighteen precise actions that are actuated by the computer-controlled transmission drive system in the surgeon's wand. The chart of FIG. 41 lists these eighteen actions in the left column. The transmission drive utilized for each action is shown in the middle column; these six transmission drives are the same as those used in the Type I anastomosis described earlier. The right column of FIG. 41 indicates the procedure step number in which each applicator action occurs.

All these steps and actions have been described in FIGS. 33 through 40. The overall process can be described as follows: (1) the surgeon performs Steps 0, 1, 2, 3 and 4, (2) at the surgeon's command, the computer takes over and the A/A 9 performs the first sequence of eleven actions which execute Steps 5 through 13, (3) the surgeon performs Step 14, (4) at the surgeon's command, the computer takes over again and the A/A 9 performs the second sequence of seven actions which accomplish Steps 15 through 20 and (5) the surgeon removes the A/A 9 from his wand and returns it to its container.

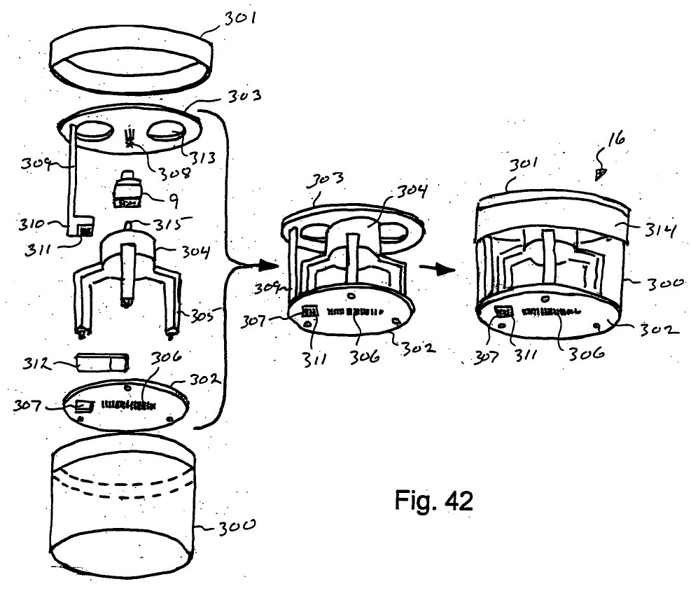

The actions described above for performing anastomoses utilizing the SAS device of the present invention require the Appliance/Applicator 9 which is provided in the form of an appliance/applicator kit 16 that is supplied to the surgeon on the appliance/applicator trays. The appliance/applicator kit 16 of the present invention is a unique apparatus and is illustrated in FIG. 42 which shows an exploded view of the components of the kit.

The appliance/applicator kit 16 comprises a container 300 with a removable lid 301 and before use is sealed with a hermetic seal 314. On the top side of the lid 301 is a label identifying the enclosed appliance/applicator 9 according to its type and critical dimensions. Within the container are housed the appliance/applicator 9, an opaque bar code disk 302 and a retainer disk 303. The appliance/applicator 9 is supported on a cradle 304, a positioning pin 315 being provided on the top of the cradle to locate the appliance/applicator 9 centrally thereon. Elongated legs 305 support the cradle above bar code disk 302 which rests at the bottom of the container 300. On the lower side of the bar code disk 302 is a primary bar code 306 providing information corresponding to that on the label applied to the lid and in a form readable by an appropriate bar code scanner 17 in the SAS. Adjacent to the primary bar code 306 is a supplemental bar code aperture 307. On the upper side of the bar code disk 302 and extending over the supplemental bar code aperture 307 is an opaque flexible flap 312, the purpose of which will be explained later.

The retainer disk 303 fits over the top of the container 300 between the top edge of the container 300 and the underside of the lid 301, its diameter being sufficient to prevent it from fitting within the container 300. Finger holes 313 are provided in the retainer disk 303 to facilitate its removal for access to the A/A 9. Depending from the underside of the retainer disk 303 at a substantially central location is a transmission restraint pin 308 which is inserted into the transmission housing 134 of the A/A 9 where it serves to restrain the A/A drive transmission 137 in their initial positions for engagement with the applicator drive gears 33 at the end of the stylus 26. Also extending from the underside of the retainer disk 303 substantially adjacent to the edge thereof is a supplemental bar code strut 309, which extends downward to the bar code disk 302. At the lower end of the strut 309 is a tab 310 bearing a supplemental bar code 311. When the appliance/applicator kit is assembled, the tab 310 is positioned within the supplemental bar code aperture 307 so that the supplemental bar code 311 is readable along with the primary bar code 306. The opaque flexible flap 312 extends over the back of the supplemental bar code tab 310.

When the appliance/applicator kit 16 is assembled and before the A/A 9 is removed for use, the opaque flexible flap 312 extends over the supplemental bar code tab 310. In order to remove the A/A 9 the retainer disk 303 is first removed thus exposing the A/A 9. In so doing, the supplemental bar code tab 310 is removed from between the supplemental bar code aperture 307 and the opaque flexible flap 312 which permits the flap 312 to cover the aperture 307. After use, when the A/A 9 is returned to the container 303 and the container 303 is returned to the appliance applicator tray 10, the opaque flexible flap prevents the supplemental bar code 311 from being scanned by the bar code scanner thereby indicating to the computer that the A/A 9 for that location has been used.

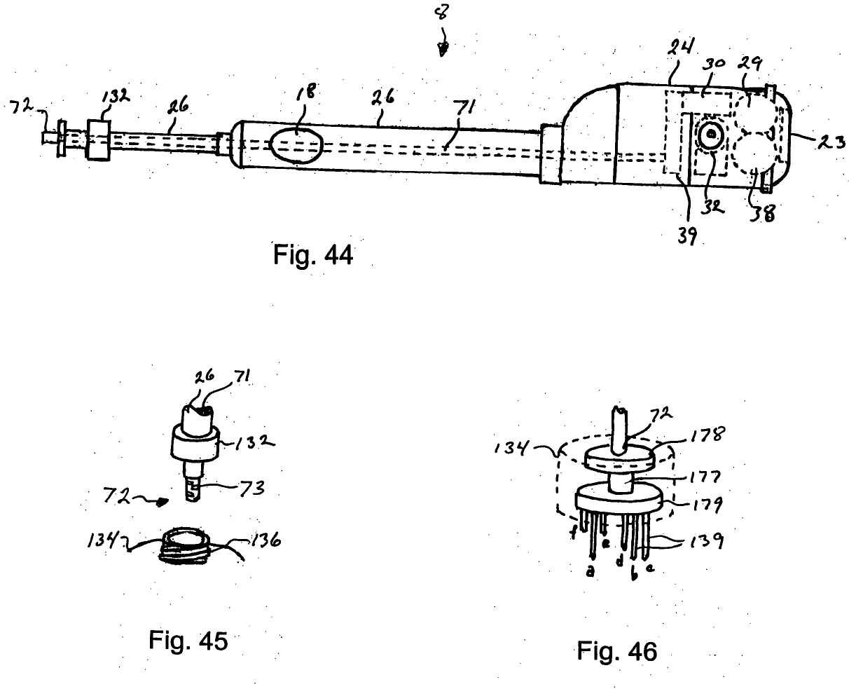

Alternative embodiments of the appliance/applicator 9 and wand 8 are illustrated in FIGS. 44, 45 and 46 in which the drive mechanism comprising the linear and rotary actuators 35, 36, transmission 28, concentric drive shafts 31 and appliance drive gears 33 in the wand 8 are eliminated. Instead all mechanical drive mechanisms are located in the transmission housing 134 of the appliance/applicator 9 and are activated in response to electronic signals transmitted via the control electronics 39 in the wand 8. Communication from the wand 8 to the appliance/actuator 9 is through wire, infra-red, or optical means along conduit 71. Instead of appliance drive gears 33 at the end of stylus 26, conduit 71 terminates in connector 72 which is appropriate to the type of communication means used. In this embodiment, power to the appliance/applicator 9 is preferably provided by the batteries 38 in wand 8 and they may be rechargeable or replaceable. Power from the batteries 38 is routed along a conductor through conduit 71 to connector 72 which is provided with contacts 73 to engage corresponding contacts in the appliance/applicator 9 when connected to the end of the stylus 26. Alternatively, each appliance/applicator 9 may be provided with its own battery which is activated when it is removed from the kit 16 and connected to the end of the stylus 26. The actual power requirement of the appliance/applicator 9 is low and the duration short such that common button type batteries, such as those used in watches and hearing aids, would be suitable.

Turning now to the appliance/applicator 9, as shown in FIG. 46, the appliance/applicator 9 of this embodiment houses a drive means 177, electronic control circuit 178 and transmission mechanism 179 in the transmission housing 134. Output drive shafts 139a-f extend out of the transmission housing 134 into the appliance and manipulation housing 135 to drive the various elements therein as previously described. Electronic control circuit 178 receives connector 72 and is provided with contacts corresponding to contacts 73 on connector 72 to receive signals and power form wand 8. Power is routed to the drive means 177, the operation of which is regulated by electronic control circuit 178. Drive means 177 in turn drives transmission mechanism 179 in response to signals provided by electronic control circuit 178.

Transmission mechanism houses the upper ends of output drive shafts 139a-f with spur gears 138. In order to ensure that engagement of drive means is made with only one spur gear at any one time, spur gears 138 are positioned at different vertical positions within transmission mechanism 179 and drive means 177 comprises a rotary and linear actuator similar to the principal embodiment. In this manner, the linear actuator serves to position drive means 177 at vertical positions corresponding to each spur gear 138 of output drive shafts 139a-f so that the rotary actuator is engaged with the particular spur gear of the particular shaft 139a-f corresponding to the step to be performed in an anastomosis as previously described.

While the invention has been described with respect to certain specific embodiments, it will be appreciated that many modifications and changes may be made by those skilled in the art without departing from the spirit of the invention. It is intended, therefore, that all such modifications and changes are within the true spirit and scope of the invention as recited in the following claims.

1. A surgical anastomosis apparatus comprising:
a mobile console housing a power supply, computer control means and manipulator means,
a plurality of self contained anastomosis appliance and applicator assemblies attachable to said manipulator means and actuated by said computer control means, whereby
said anastomosis appliance and applicator assemblies are capable of drawing together and securing two prepared blood vessels to form an anastomosis.

2. The surgical anastomosis apparatus of claim 1 wherein said mobile console comprises,
a transport cart containing said power supply and computer control means, and further containing an automated code scanner,
at least one tray holding a plurality of said anastomosis appliance and applicator assemblies over said automated code scanner,
a vertically extendable mast assembly extendable above said at least one tray,
a positioning arm extendable horizontally from said mast assembly and rotatable horizontally relative thereto,
a working arm connected to said positioning arm at an end remote from said mast assembly and rotatable and pivotable relative to said positioning arm, and
a manipulatable wand connected to said working arm remote from said positioning arm, said wand being rotatable and pivotable relative to said working arm and having an end adapted to removably receive said anastomosis appliance and applicator assemblies.

3. The surgical anastomosis apparatus of claim 2 further comprising communication means between said computer control means and said manipulatable wand whereby said computer control means regulates operation of actuators within said wand, which actuators, in turn, operate said appliance and applicator assemblies.

4. The surgical anastomosis apparatus of claim 3 further comprising a primary control panel on said cart providing communication with said computer control means.

5. The surgical anastomosis apparatus of claim 4 further comprising at least one secondary control panel attached to said positioning arm and providing communication with said computer control means.

6. The surgical anastomosis apparatus of claim 5 wherein said mast assembly comprises at least two telescoping sections capable of retracting into and extending upwards from said cart, and a drive mechanism adapted to extend and retract said mast assembly in response to commands entered at said primary or secondary control panels.

7. The surgical anastomosis apparatus of claim 6 further comprising a brake mechanism associated with said mast assembly and drive mechanism whereby said mast is positively retained in position when extended.

8. The surgical anastomosis apparatus of claim 7 wherein said at least one tray and said automated code scanner are attached to said mast assembly and are raised to an operational height when said mast assembly is extended.

9. The surgical anastomosis apparatus of claim 8 wherein said positioning arm comprises at least two relatively telescopable sections, a first section extending horizontally from the upper end of said mast assembly and connected thereto by a fitting whereby said positioning arm is rotatable about the longitudinal axis of said mast assembly, said fitting being lockable to maintain said positioning arm in a desired position, and a second section telescopable within said first section, said positioning arm having locking means to secure said second section relative to said first section.

10. The surgical anastomosis apparatus of claim 9 wherein said working arm comprises a first segment connected to the free end of said positioning arm by a two-degree of freedom rotating joint, and a second segment telescopably slidable within said first segment, wherein said first segment houses a counterbalance means attached to said second segment and adapted to automatically and dynamically balance said working arm and said manipulatable wand when said second segment is extended or telescoped during use, said second segment having a two-degree of freedom rotating joint at the distal end thereof, said joint having a receptacle with a quick connect fitting whereby said manipulatable wand is attached to said second segment.

11. The surgical anastomosis apparatus of claim 10 wherein said counterbalance means comprises a linear dynamic balance system comprising fixed and movable counterweights, said movable counterweights being part of a pulley system whereby said movable counterweights respond to telescoping action of said second segment within said first segment to maintain said working arm and attached manipulatable wand in linear balance at any extension of said second segment.

12. The surgical anastomosis apparatus of claim 11 wherein said manipulatable wand comprises a functional unit housing two actuators, a mechanical transmission assembly, power supply and electronic control means and communication transmission means, a handle connected to and extending linearly from said functional unit and housing mechanical transfer means and having a switch means connected to said electronic control means and communication transmission means providing remote control and activation means between said wand and said computer control means, and a stylus connected to the proximal end of said handle and having an interface adapted to receive and transfer mechanical motion from said mechanical transfer means to said anastomosis appliance and applicator assemblies, said apparatus further comprising releasable brake means associated with all joints of said working arm, said brake means being operable in response to said switch means on said wand whereby release of said brake means allows complete movement of said arms and said wand through all degrees of freedom and activation of said brake means fixes said joints thereby holding said wand and said anastomosis appliance and applicator assemblies in a fixed position.

13. The surgical anastomosis apparatus of claim 12 wherein said two actuators comprise a linear actuator and a rotary actuator, said actuators being mechanically connected to said mechanical transmission assembly whereby mechnical motion generated by said actuators is conveyed to said transmission assembly, said transmission assembly being mechanically connected to said mechanical transfer means whereby rotary motion generated by said rotary actuator is transferred by said transmission assembly and said mechanical transfer means to said interface and, thereby, to said anastomosis appliance and applicator assemblies.

14. The surgical anastomosis apparatus of claim 13 wherein said mechanical transfer means comprises a plurality of concentric tubular shafts independently axially rotatable within said handle and said stylus, said transmission assembly comprises a first set of gears corresponding to said shafts, each shaft having one gear of said first set fixedly attached to its distal end, said interface comprising a second set of gears corresponding to said shafts, each shaft having one gear of second set attached to its proximal end, whereby said first set of gears and said second set of gears each form a linear arrangement at each end of said shafts that is a mirror image of the other, said transmission assembly further comprising a pinion gear mounted on an axle rotatable by said rotary actuator and linearly translatable relative to said first set of gears by said linear actuator whereby said pinion gear is adapted to selectively engage and rotate each of said first set of gears and their corresponding shafts in accordance with a particular sequence dictated by the computer control means for a particular anastomosis appliance and applicator assembly.

15. The surgical anastomosis apparatus of claim 14 wherein said transmission assembly further comprises a snubber assembly cooperatively associated with said pinion gear which snubber assembly engages all of said first set of gears except the gear engaged by said pinion gear whereby only one shaft is capable of rotation at any one time during operation of said transmission assembly.

16. The surgical anastomosis apparatus of claim 15 wherein said anastomosis appliance and applicator assemblies each comprise a housing removably attachable to said stylus and adapted to receive blood vessels for anastomosis, said housing having a transmission portion and an appliance applicator portion, wherein said transmission portion is adapted to receive said interface of said stylus and comprises a mechanical mechanism adapted to transmit rotary motion from said second set of gears to specific elements of said appliance applicator portion, and wherein said appliance applicator portion comprises a sequentially operable mechanism comprising inflatable means adapted to evert blood vessel openings whereby blood vessel intima is exposed, a plurality of gripping connectors adapted to engage and hold said everted blood vessel openings, and translation means adapted to bring said everted blood vessels into intima-to-intima contact and engage said gripping connectors whereby said anastomosis is performed in a substantially automated procedure.

17. An anastomosis apparatus comprising a manipulatable wand adapted to operate a self contained anastomosis appliance and applicator attached thereto, a wand positioning means adapted to provide gross and fine positioning of said wand and anastomosis appliance and applicator relative to blood vessels to be anastomosed, and computer control means adapted to sequentially actuate said anastomosis appliance and applicator in response to data input relative to the type of anastomosis to be performed, wherein said wand comprises a functional unit housing a power supply, an electronic control means and a mechanical actuator means, a transmission means, and an anastomosis appliance and applicator interface.

18. The anastomosis apparatus of claim 17 wherein said mechnical actuator means comprises a linear actuator and a rotary actuator operated by said power supply in response to signals generated by said electronic control means, said transmission means comprises a plurality of concentric shafts each independently axially rotatable by said rotary actuator operating through a first transmission mechanism capable of engaging only one shaft at a time, said transmission mechnism being controlled by said linear actuator, said transmission means terminating in said interface comprising a second transmission mechanism adapted to transfer rotary motion of said shafts to said anastomosis appliance and applicator.

19. The anastomosis apparatus of claim 18 further comprising data input means whereby data relating to type of anastomosis to be performed, size of vessels, and type of anastomosis appliance and applicator to be used is entered into said computer control means whereby control signals relating to an operation sequence are generated by said electronic control means and said signals regulate the operation of said linear and rotary actuators, and communication means between said computer control means and said electronic control means.

20. The anastomosis apparatus of claim 19, wherein said first transmission mechanism comprises a plurality of equal diameter gears, each gear being secured to a first end of each shaft wherein each shaft from the outermost shaft inward is longer than the preceding shaft by an amount sufficient to permit the gear thereon to be engaged apart from the other gears, each gear being mounted on the end of its shaft and having an aperture therethrough corresponding to the inner diameter of said shaft whereby said successive concentric shafts are able to pass therethrough and freely rotate therein, whereby said gears form a linear array; said first transmission mechanism further comprising a pinion gear lateral to said linear gear array and adapted to individually engage said gears, said pinion gear being mounted on a shaft for rotation by said rotary actuator and for linear translation relative to said linear array by said linear actuator, whereby, in response to said electronic control means, said linear actuator causes said pinion gear to move into engagement with one of said linear array gears and said rotary actuator causes said pinion gear to rotate whereupon said engaged linear array gear and its shaft are rotated.

21. The anastomosis apparatus of claim 20 wherein said first transmission mechanism further comprises a snubber assembly associated with said pinion gear, said snubber assembly comprising a vane capable of engaging all of said linear array gears except the gear engaged by said pinion gear, said snubber assembly being linearly translatable concurrently with said pinion gear by said linear actuator, whereby said linear array gears not engaged by said pinion gear are prevented from rotating.

22. The anastomosis apparatus of claim 21, wherein said second transmission mechanism comprises a plurality of equal diameter gears identical to said equal diameter gears of said first transmission mechanism, each gear being secured to a second end of each shaft in a manner identical to said first transmission mechanism, whereby said second transmission mechanism comprises a mirror image of said linear array of gears in said first transmission mechanism, and wherein said second transmission mechanism is adapted to be received in and transmit rotational motion to said anastomosis appliance and applicator.

23. The anastomosis apparatus of claim 22 further comprising manual control means on said wand to activate and deactivate said electronic control means and said mechanical actuator means.

24. An anastomosis apparatus comprising a self contained anastomosis appliance and applicator, a manipulatable wand adapted to operate said self contained anastomosis appliance and applicator removably attachable thereto, a wand positioning means adapted to provide gross and fine positioning of said wand and anastomosis appliance and applicator relative to said vessels to be anastomosed, and computer control means adapted to sequentially actuate said anastomosis appliance and applicator in response to data input relative to the type of anastomosis to be performed, wherein said wand comprises a power supply, mechanical actuator means, transmission means, and an anastomosis appliance and applicator interface adapted to engage said appliance and applicator and transfer mechanical motion from said mechanical actuator means, said interface comprising a plurality of gears, wherein said anastomosis appliance and applicator comprises a housing having a transmission portion and an appliance/applicator portion, said transmission portion adapted to receive said interface and comprising a plurality of gears engagable with said gears of said interface, said appliance/applicator portion being adapted to receive portions of blood vessels to be anastomosed and comprising a sequentially operable mechanism comprising inflatable means adapted to evert blood vessel openings whereby blood vessel intima is exposed, and anastomosis connecting means comprising a plurality of gripping connectors adapted to engage and hold said everted blood vessel openings, holders for said gripping connectors and translation means adapted to bring said everted blood vessels into intima-to-intima contact and engage said gripping connectors whereby said anastomosis is performed in a substantially automated procedure.

25. The anastomosis apparatus of claim 24 wherein said transmission portion further comprises shaft members attached to said gears and extending into said appliance applicator portion, said shaft members having means adapted to engage and drive said sequentially operable mechanisms in said appliance applicator portion in response to rotation of said gears by said interface.

26. The anastomosis apparatus of claim 25 wherein said inflatable means comprises first and second balloon structures removably positionable between blood vessels to be anastomosed, said balloon structures each comprising a balloon and holder assembly wherein said balloon is attached to and inflatable away from said holder to evert said blood vessels, said inflatable means further comprising an inflation syringe fluidly connected to said balloons whereby said balloons are sequentially inflated and deflated, said balloon structures being removably positioned and said syringe being driven by said shaft members extending from said transmission portion.

27. The anastomosis apparatus of claim 26 wherein said first and second balloon structures are identical and are removably positionable between blood vessel ends to be anastomosed in a Type I anastomosis.

28. The anastomosis apparatus of claim 26 wherein said first and second balloon structures are different and are removably positionable between blood vessels being anastomosed in a Type III anastomosis.

29. The anastomosis apparatus of claim 26 wherein said anastomosis connecting means comprises first and second connector holder/ejector assemblies through which said blood vessels are inserted for anastomosis, said assemblies each comprising a substantially annular gripping connector holder, a cooperating substantially annular gripping connector ejector, a plurality of two part cooperating gripping connectors, said connectors of one part being held in said first holder and said connectors of the other part being held in said second holder, and a translation mechanism, wherein said connectors are adapted to grip and hold said everted blood vessels, said holder/ejector assemblies are adapted to translate laterally from a first spaced apart position following deflation of said balloons to a second position whereby said blood vessels are brought into contact with each other and said connectors are connected, and said ejectors are activated to eject said connectors from said holders whereby said anastomosis is completed and held by said connectors and said holder/ejector assemblies are translated away from said anastomosis to a final position.

30. The anastomosis apparatus of claim 29 wherein said substantially annular holders and ejectors are separable in at least one location through their annular peripheries whereby said holder/ejector assemblies are removable from around said anastomosed blood vessels.

31. The anastomosis apparatus of claim 30 wherein said translation mechanism comprises a plurality of gear driven screw shafts operating through said holder/ejector assemblies and driven by said shafts extending from said transmission portion.

32. The anastomosis apparatus of claim 31 wherein each holder and ejector comprises a semicircular and two quarter-circular sections with each quarter-circular section being hinged to one end of said semicircular section, said appliance applicator portion of said housing having guide means therein cooperating with said holder/ejector assemblies whereby said quarter-circular sections are maintained in engagement with each other and with said semicircular section between said first position and said second position and wherein translation to said final position removes said holder/ejector assemblies from cooperation with said guide means whereby said quarter-circular sections are free to separate whereby said holders and ejectors are permitted to open thereby permitting removal of said apparatus from said blood vessels.

33. The anastomosis apparatus of claim 31 wherein each holder/ejector assembly comprises an inner ring and an outer ring, said inner ring being segmented at four places thereby forming two end segments and two side segments, whereby each segment is separately translatable toward said outer ring, said inner ring further comprises means to releasably hold said connectors, said outer ring having at least one end hinged so as to be openable upon completion of an anastomosis and carrying ejector means whereby said connectors are ejected from said inner ring segments upon translation of said segments toward said outer ring, said outer ring further comprising translation means activated by said shafts extending from said transmission portion whereby said inner ring segments are translated.

34. The anastomosis apparatus of claim 33 wherein said first balloon structure further comprises a punch assembly having a punch blade and a cooperating anvil extending below said balloon, said anvil further comprising a strut whereby said anvil is extendable and retractable relative to said blade, said anvil being adapted for insertion into a blood vessel through an incision in a side wall thereof, whereby said anvil is retractable toward said blade thereby severing and trapping a portion of said blood vessel side wall and creating an arterio/venotomy for eversion by said balloon, said blade and anvil having a substantially oval shape.

35. The anastomosis apparatus of claim 34 wherein said first balloon structure further comprises means for extension and retraction of said structure through the opening formed by said inner ring.

36. The anastomosis apparatus of claim 24 wherein said gripping connectors comprise a body, at least one tine extending from said body so as to engage a blood vessel wall, and a connecting structure whereby two oppositely positioned gripping connectors are joined.

37. The anastomosis apparatus of claim 36 wherein said gripping connector body comprises a forward face portion having a curvature with a radius corresponding to the thickness of a blood vessel wall to be anastomosed, said at least one tine extends outward and upward from said face portion a distance of from ⅓ to ⅔ the thickness of said blood vessel wall, and said connecting structure extends forward from the upper edge of said forward face portion over said at least one tine.

38. The anastomosis apparatus of claim 37 wherein said connecting structure on one of a pair of said gripping connectors comprises a male connector element and said connecting structure on the other of said pair of gripping connectors comprises a female connector element and each connector element comprises a cooperating detent means whereby said connectors are secured together when said connecting structures are joined.

39. The anastomosis apparatus of claim 37 wherein said connecting structure on one of a pair of said gripping connectors comprises an extension of said body extending forward of said curved face portion and having a locator pin thereon, and a clip member extending over said extension beyond said locator pin, said connecting structure on the other of said pair of gripping connectors comprises a hole in an upper portion of said forward face portion and a groove in a back surface of said body adapted to receive the end of said clip member.

40. The anastomosis apparatus of claim 37 wherein said connecting structure comprises mirror image pairs of interdigitating elongated extensions on each gripping connector, said extensions having cooperating detent means whereby said extensions interdigitate and are held together when cooperating pairs of gripping connectors are joined.

41. The anastomosis apparatus of claim 36 wherein said gripping connectors are adapted to connect blood vessels in a Type III anastomosis and consist of a first connector comprising an elongated body having a first end with said at least one tine extending outward and downward therefrom a distance of from ⅓ to ⅔ the thickness of a blood vessel wall, a lower face having a curvature with a radius corresponding to the thickness of said blood vessel wall and a second end having a male connecting structure extending downward therefrom, and a second connector comprising a body having a curved face with a radius corresponding to the thickness of blood vessel, said at least one tine extending outward and upward from said curved face and a female connecting structure comprising an aperture extending downward through said body from an upper surface thereof, adapted to receive said male connecting structure, said male and female connecting structures having cooperating detent means, and whereby said first and second connectors fit together such that said tines assume a substantially 90° relationship.

42. A self contained anastomosis appliance and applicator comprising a housing having a transmission portion and an appliance applicator portion, said transmission portion containing a drive mechanism adapted to operate said appliance applicator and adapted to receive and connect to a remote power source, said appliance applicator portion having means to receive portions of blood vessels to be anastomosed and comprising a sequentially operable mechanism comprising inflatable means adapted to evert blood vessel openings whereby blood vessel intima is exposed, anastomosis connecting means comprising a plurality of opposed pairs of gripping connectors adapted to engage and hold said everted blood vessel openings, holders for said gripping connectors, translation means adapted to bring said everted blood vessels into intima-to-intima contact and engage said opposed pairs of gripping connectors whereby said opposed pairs of gripping connectors are joined in a secure manner and and said blood vessls are held together whereby said anastomosis is performed.

43. The self contained anastomosis appliance and applicator of claim 42 wherein said inflatable means comprises first and second balloon structures removable positionable between said blood vessels to be anastomosed, said balloon structures each comprising an inflatable balloon and a balloon holder wherein said balloon is attached to and inflatable away from said holder into said blood vessel, said balloon being adapted, when inflated, to engage a prepared edge of a wall of said blood vessel whereby said prepared edge is everted, said inflatable means further comprising an inflation syringe fluidly connected to said balloons and including means to sequentially and individually inflate and deflate said balloons, said balloon holders further comprising mechanical means whereby said first and second balloon structures are positioned between said blood vessels, said syringe is actuated to sequentially inflate and deflate said balloons, and said first and second balloon structures are withdrawn from between said blood vessels wherein said mechanical means is driven by said drive mechanism in said transmission portion.

44. The self contained anastomosis appliance and applicator of claim 43 wherein said anastomosis connecting means comprises first and second connector holder/ejector assemblies through which said blood vessels are inserted for anastomosis, said assemblies each comprising a substantially annular gripping connector holder, a cooperating substantially annular gripping connector ejector, a plurality of said opposed pairs of gripping connectors, one of each pair of said connectors being held in said first holder and the other of each pair of said connectors being held in said second holder, and a translation mechanism, wherein said connectors are adapted to grip and hold said everted blood vessels following withdrawal of said first and second balloon structures, said holder/ejector assemblies are adapted to translate laterally from a first spaced apart position to a second position whereby said blood vessels are brought into contact with each other and said pairs of said connectors are connected, and said ejectors are activated to eject said connectors from said holders whereby said anastomosis is completed and held by said connectors and said holder/ejector assemblies are translated away from said anastomosis to a final position.

45. The self contained anastomosis appliance and applicator of claim 44 wherein said substantially annular holders and ejectors are separable in at least one location through their annular peripheries whereby said holder/ejector assemblies are removable from around said anastomosed blood vessels.

46. The self contained anastomosis appliance and applicator of claim 45 wherein said translation mechanism comprises a plurality of gear driven screw shafts operating through said holder/ejector assemblies and driven by said drive mechanism in said transmission portion.

47. The self contained anastomosis appliance and applicator of claim 46 wherein each holder and ejector comprises a semicircular and two quarter-circular sections with each quarter-circular section being hinged to one end of said semicircular section, said appliance applicator portion of said housing having guide means therein cooperating with said holder/ejector assemblies whereby said quarter-circular sections are maintained in engagement with each other and with said semicircular section between said first position and said second position and wherein translation to said final position removes said holder/ejector assemblies from cooperation with said guide means whereby said quarter-circular sections are free to separate whereby said holders and ejectors are permitted to open thereby permitting removal of said apparatus from said blood vessels.

48. The self contained anastomosis appliance and applicator of claim 46 wherein each holder/ejector assembly comprises an inner ring and an outer ring, said inner ring being segmented at four places thereby forming two end segments and two side segments, whereby each segment is separately translatable toward said outer ring, said inner ring further comprises means to releasably hold said connectors, said outer ring having at least one end hinged so as to be openable upon completion of an anastomosis and carrying ejector means whereby said connectors are ejected from said inner ring segments upon translation of said segments toward said outer ring, said outer ring further comprising translation means activated by said drive mechanism in said transmission portion whereby said inner ring segments are translated.

49. The self contained anastomosis appliance and applicator of claim 48 wherein said first balloon structure further comprises a punch assembly having a punch blade and a cooperating anvil extending below said balloon, said anvil further comprising a strut whereby said anvil is extendable and retractable relative to said blade, said anvil being adapted for insertion into a blood vessel through an incision in a side wall thereof, whereby said anvil is retractable toward said blade thereby severing and trapping a portion of said blood vessel side wall and creating an arterio/venotomy for eversion by said balloon, said blade and anvil having a substantially oval shape.

50. The self contained anastomosis appliance and applicator of claim 49 wherein said first balloon structure further comprises means for extension and retraction of said structure through the opening formed by said inner ring.

51. The anastomosis apparatus of claim 46 wherein said gripping connectors comprise a body, at least one tine extending from said body so as to engage a blood vessel wall, and a connecting structure whereby two oppositely positioned gripping connectors are joined.

52. The anastomosis apparatus of claim 51 wherein said gripping connector body comprises a forward face portion having a curvature with a radius corresponding to the thickness of a blood vessel wall to be anastomosed, said at least one tine extends outward and upward from said face portion a distance of from ⅓ to ⅔ the thickness of said blood vessel wall, and said connecting structure extends forward from the upper edge of said forward face portion over said at least one tine.

53. The anastomosis apparatus of claim 52 wherein said connecting structure on one of a pair of said gripping connectors comprises a male connector element and said connecting structure on the other of said pair of gripping connectors comprises a female connector element and each connector element comprises a cooperating detent means whereby said connectors are secured together when said connecting structures are joined.

54. The anastomosis apparatus of claim 52 wherein said connecting structure on one of a pair of said gripping connectors comprises an extension of said body extending forward of said curved face portion and having a locator pin thereon, and a clip member extending over said extension beyond said locator pin, said connecting structure on the other of said pair of gripping connectors comprises a hole in an upper portion of said forward face portion and a groove in a back surface of said body adapted to receive the end of said clip member.

55. The anastomosis apparatus of claim 52 wherein said connecting structure comprises mirror image pairs of interdigitating elongated extensions on each gripping connector, said extensions having cooperating detent means whereby said extensions interdigitate and are held together when cooperating pairs of gripping connectors are joined.

56. The anastomosis apparatus of claim 51 wherein said gripping connectors are adapted to connect blood vessels in a Type III anastomosis and consist of a first connector comprising an elongated body having a first end with said at least one tine extending outward and downward therefrom a distance of from ⅓ to ⅔ the thickness of a blood vessel wall, a lower face having a curvature with a radius corresponding to the thickness of said blood vessel wall and a second end having a male connecting structure extending downward therefrom, and a second connector comprising a body having a curved face with a radius corresponding to the thickness of blood vessel, said at least one tine extending outward and upward from said curved face and a female connecting structure comprising an aperture extending downward through said body from an upper surface thereof, adapted to receive said male connecting structure, said male and female connecting structures having cooperating detent means, and whereby said first and second connectors fit together such that said tines assume a substantially 90° relationship.

57. An anastomosis apparatus comprising a manipulatable wand adapted to operate a self contained anastomosis appliance and applicator attached thereto, a wand positioning means adapted to provide gross and fine positioning of said wand and anastomosis appliance and applicator relative to blood vessels to be anastomosed, and computer control means adapted to sequentially actuate said anastomosis appliance and applicator in response to data input relative to the type of anastomosis to be performed, wherein said wand comprises a functional unit housing a power supply, an electronic control means, an anastomosis appliance and applicator interface and at least one anastomosis appliance and applicator engagable with said interface.

58. The anastomosis apparatus of claim 57, wherein said anastomosis appliance and applicator comprises a housing having a transmission portion and an appliance applicator portion, said transmission portion adapted to receive said interface and said appliance applicator portion adapted to receive portions of blood vessels to be anastomosed and comprising a sequentially operable mechanism comprising inflatable means adapted to evert blood vessel openings whereby blood vessel intima is exposed, and anastomosis connecting means comprising a plurality of gripping connectors adapted to engage and hold said everted blood vessel openings, holders for said gripping connectors and translation means adapted to bring said everted blood vessels into intima-to-intima contact and engage said gripping connectors whereby said anastomosis is performed in a substantially automated procedure.

59. The anastomosis apparatus of claim 58 wherein said transmission portion further comprises a drive means and a transmission means, said transmission means comprising a plurality of shaft members extending into said appliance applicator portion, each of said shaft members having a first end in said transmission portion having a gear adapted for sequential engagement by said drive means and a second end in said appliance applicator portion having means adapted to engage and drive said sequentially operable mechanisms in said appliance applicator portion in response to rotation of said shafts said drive means.

60. The anastomosis apparatus of claim 59 wherein said drive means comprises a linear actuator and a rotary actuator operating in response to said electronic control means, whereby said linear actuator operates to sequentially engage said rotary actuator with said gears on said first ends of said shaft members.
